(19) United States
(12) Patent Application Publication
(10) Pub. No.: US 2024/0263239 A1
(43) Pub. Date: Aug. 8, 2024

(54) SINGLE-CELL PROFILING OF CHROMATIN OCCUPANCY AND RNA SEQUENCING

(71) Applicant: The United States of America, as represented by the Secretary, Dept. of Health and Human Services, Bethesda, MD (US)

(72) Inventor: Keji Zhao, Kensington, MD (US)

(21) Appl. No.: 18/036,392

(22) PCT Filed: Nov. 10, 2021
(86) PCT No.: PCT/US2021/058809

§ 371 Date: May 10, 2023

Related U.S. Application Data
(60) Provisional application No. 63/111,951, filed on Nov. 10, 2020.

(51) Int. Cl. C12Q 1/6886; C12N 9/12; C12Q 1/6806; C12Q 1/6841

(52) U.S. Cl. CPC C12Q 1/6886; C12N 9/1264; C12Q 1/6806; C12Q 1/6841; C12Q 2600/118; C12Q 2600/156; C12Q 2600/158

(57) ABSTRACT

Compositions and methods for determining and identifying both chromatin occupancy and transcriptome simultaneously in the same single cell.

### CROSS REFERENCE TO RELATED APPLICATIONS

This Application claims the benefit of U.S. Provisional Application 63/111,951 filed on Nov. 10, 2020. The entire contents of this application is incorporated herein by reference in its entirety.

### FIELD

In one aspect, methods and compositions are provided for simultaneously profiling genome-wide chromatin protein binding or histone modification marks and RNA expression in the same cell.

### BACKGROUND

Gene expression exhibits remarkable cellular heterogeneity, which may be influenced by multiple factors including different aspects of chromatin modifications (Corces, M. R. et al.

(2016) Lineage-specific and single-cell chromatin accessibility charts human hematopoiesis and leukemia evolution. Nat Genet 48, 1193-1203, doi: 10.1038/ng.3646; Cheung, P. et al. (2018) Single-Cell Chromatin Modification Profiling Reveals Increased Epigenetic Variations with Aging. Cell 173, 1385-1397 e1314, doi: 10.1016/j.cell.2018.03.079). In the past few years, several assays measuring different aspects of chromatin states at a single-cell resolution have been developed. These include Droplet-based single cell ChIP-seq15, Tn5-based chromatin accessibility assays (ATAC-seq) (Buenrostro, J. D. et al. (2015) Single-cell chromatin accessibility reveals principles of regulatory variation. Nature 523, 486-490, doi:10.1038/nature14590. Cusanovich, D. A. et al. (2015) Multiplex single cell profiling of chromatin accessibility by combinatorial cellular indexing. Science 348, 910-914, doi: 10.1126/science.aab1601). DNase I hypersensitivity assay(DNase-seq) (Jin, W. et al. (2015) Genome-wide detection of DNase I hypersensitive sites in single cells and FFPE tissue samples. Nature 528, 142-146, doi: 10.1038/nature15740), MNase-based nucleosome position and chromatin accessibility assay (scMNase-seq) (Lai, B. et al. (2018) Principles of nucleosome organization revealed by single-cell micrococcal nuclease sequencing. Nature 562, 281-285, doi: 10.1038/s41586-018-0567-3), immunocleavage-based histone modification assays (Cut&Run, scChIC-seq) (Ku, W. L. et al. Single-cell chromatin immunocleavage sequencing (scChIC-seq) to profile histone modification. Nat Methods 16, 323-325, doi: 10.1038/s41592-019-0361-7 (2019). Skene, P. J. & Henikoff, S. An efficient targeted nuclease strategy for high-resolution mapping of DNA binding sites. Elife 6, doi: 10.7554/eLife.21856 (2017). Hainer, S. J., Boskovic, A., McCannell, K. N., Rando, O. J. & Fazzio, T. G. (2019) Profiling of Pluripotency Factors in Single Cells and Early Embryos. Cell 177, 1319-1329 e1311, doi: 10.1016/j.cell.2019.03.014), antibody-guided Tn5 chromatin tagging assays (ACT-seq, Cut&Tag, CoBATCH) (Carter, B. et al. Mapping histone modifications in low cell number and single cells using antibody-guided chromatin tagmentation (ACT-seq). Nature communications 10, 1-5 (2019). Wang, Q. et al. CoBATCH for High-Throughput Single-Cell Epigenomic Profiling. Mol Cell 76, 206-216 e207, doi: 10.1016/j.molcel.2019.07.015 (2019). Kaya-Okur, H. S. et al. (2019) CUT&Tag for efficient epigenomic profiling of small samples and single cells. Nat Commun 10, 1930, doi: 10.1038/s41467-019-09982-5), and NOMe-seq assay (Pott, S. (2017) Simultaneous measurement of chromatin accessibility, DNA methylation, and nucleosome phasing in single cells. Elife 6, doi: 10.7554/eLife.23203). These assays measure one or more aspects of chromatin states and provide data on cellular heterogeneity in chromatin but do not directly measure simultaneously both RNA and chromatin or transcription factor binding in the same single cell.

### SUMMARY

In one aspect, we now provide new compositions and methods for directly measuring simultaneously both RNA and chromatin or transcription factor binding in the same single cell.

More particularly, in one preferred aspect, methods are provided for diagnosing or prognosing an illness, the methods comprising:
1) isolating and culturing cells of interest from a sample;
2) performing chromatin cleavage and subjecting the cells to reverse transcription;
3) subjecting the cells to terminal deoxynucleotidyl transferase (TdT)-mediated oligonucleotides to both cDNA and chromatin cleaved ends in the presence of an oligonucleotide adaptor; or, subjecting the cells to end repair, deoxyadenosine addition to the DNA ends, which is followed by T/A ligation of barcoded adaptors to DNA and primer-assisted ligation of the adaptors to cDNA ends);
4) pooling the cells from each reaction well and sorting or diluting the pooled cells into new wells, followed by one or more amplification steps; and,
5) subjecting the sorted cells to a library construction and sequencing; thereby, simultaneously profiling of chromatin occupancy and RNA in a single cell. In preferred aspects, such methods may be utilized for simultaneous profiling of chromatin occupancy and RNA in a single cell. Suitably, in the methods, the cells are crosslinked with a fixative agent prior to chromatin cleavage

In a further aspect, methods are provided methods for diagnosing or prognosing an illness, the methods comprising:
1) subjecting the cells to nuclease mediated chromatin cleavage;
2) repairing of 5′ and 3′ ends of nucleic acid fragments by treatment with a polynucleotide kinase and exonuclease;
3) reverse transcribing the nucleic acid fragments;
4) contacting the cells with a barcode adaptor;
5) subjecting the cells to polyG tailing with a terminal deoxynucleotidyl transferase (TdT) and barcode adaptor ligation, producing a genomic library and sorting of the cells. In preferred aspects, such methods may be utilized for simultaneous profiling of chromatin occupancy and RNA in a single cell,

Suitably, excess primers are digested with an exonuclease prior to contacting cells with a barcode adapter.

Such methods are particularly useful to diagnosing cancer in a subject and may include treating a subject's biological sample according to a present method.

Additionally, the present methods are useful to identify biomarkers diagnostic or therapeutic of a cancer and may include treating a subject's biological sample in accordance with a method as disclosed herein, and thereafter administering to the subject a cancer therapeutic agent based on the identified biomarkers.

The present methods are also useful to determine cellular heterogeneity of solid tumor samples to treat cancer, any may include treating a subject's tumor sample in accordance with a method as disclose herein; determining the cellular heterogeneity of the tumor sample and, treating the subject with one or tumor specific therapeutic and/or chemotherapeutic agents. Preferably, the determination of the cellular heterogeneity of the tumor can accurately diagnose stages and nature of the tumor.

Still further, the present methods are also useful to evaluate cells, any may include the cells to a present method, thereby evaluating the cells. The cells may comprise, for example, tumor cells, stem cells, modified cells, infected cells, CAR-T cells, CAR-NK cells, transformed cells, cell lines or combinations thereof. The cells may be evaluated for epigenetic variations, transcriptomic variations, gene expression, protein expression, biomarkers or combinations thereof, among others.

Additional methods are provided are provided methods for diagnosing or prognosing an illness, including to identify and profile histone modifications in individual cells, the methods suitably comprising:
1) crosslinking cells with a cross-linking fixative agent;
2) contacting the fixed cells with a chromatin specific guided nuclease for cleaving the chromatin;
3) repairing of the nuclease cleaved ends by a polynucleotide kinase and adding of 5′-phosphates for poly nucleotide tailing and ligation; and,
4) barcoding of the nuclease cleaved sites with a barcode adaptor and pooling of the cells;
5) splitting of the cells and incubating the cells with a reverse cross-linking buffer;
6) capturing of barcoded cellular DNA fragments and index labeling of the barcoded DNA fragments by a first amplification assay to produce DNA libraries;
7) pooling and purifying the DNA libraries and poly A tailing the purified DNA libraries;
8) ligating the poly A tailed to an adaptor and purifying the ligated DNA;
9) performing a second amplification assay, isolating, purifying and sequencing the amplified fragments; thereby, identifying and profiling histone modifications in individual cells.

In certain aspects, the amplified DNA fragments from the first amplification assay are mapped to a human reference genome (UCSC hg18). In certain aspects, the mapped DNA fragments from the first amplification assay are separated into individual sets based on each barcode.

In certain aspects, the above method may be used to determine cellular heterogeneity and cellular differentiation in a subject, and include obtaining a sample from the subject and assaying the sample according to the above method. In certain aspects, the subject may be suffering from a genetic disorder, disease, neurological disease or disorders, cancer, autoimmune disease or combinations thereof.

In a further aspect, methods are provided for detecting and identifying nuclease hypersensitive sites in individual cells, and may comprise:
a) crosslinking cells with a fixative agent;
b) lysing the cells and digesting cellular DNA with a nuclease;
c) aliquoting of nuclei and ligating of chromatin DNA to a first barcode adaptor;
d) pooling of the nuclei followed by dilution and redistribution into separate plate well;
e) subjecting the DNA to reverse cross-linking, introducing a second barcode complementary to the first barcode adaptor via an amplification assay;
f) pooling of amplified DNA, ligating of the DNA to a second barcode adaptor;
g) amplifying the DNA and introducing a third barcode adaptor; and,
h) pooling and sequencing of amplified DNA; wherein,
i) sequences having the same combination of barcodes are derived from a single cell; thereby, detecting and identifying nuclease hypersensitive sites in individual cells.

In such method, the nuclease suitably may comprise: endonucleases, exonucleases, DNases, MNase or combinations thereof. Preferred barcode adaptors may comprise a nucleotide sequence having a 50% sequence identity to: acactgacgacatggttctacannnnnnnnagateggaagagcacacgtctgaactccagtcac (SEQ ID NO: 2), tgtagaaccatgtcgtcagtgtcccccccc/3ddC (SEQ ID NO: 3), gatcggaagagcgtcgtgtagggaaagagtg (SEQ ID NO: 4) or tctttccctacacgacgctcttccgatct (SEQ ID NO: 5).

In a yet further aspect, methods are provided for determining cellular heterogeneity and cellular differentiation occurring during development, a genetic condition or disease state, the methods suitably comprising:
1) contacting fixed cells with a chromatin specific guided nuclease for cleaving the chromatin;
2) repairing of the nuclease cleaved ends and labeling DNA ends with a dG polytail by Terminal Deoxynucleotidyl Transferase (TdT);
3) ligating of oligonucleotide dC adaptors by T4 ligase;
4) pooling of cells and sorting of cells;
5) amplifying and barcoding the DNA with a first barcode;
6) pooling of the cells and barcoding the DNA with a second barcode;
7) isolating, purifying and sequencing the amplified fragments; thereby,
8) identifying and profiling histone modifications in individual cells; thereby, determining cellular heterogeneity and cellular differentiation.

In a still further aspect, methods are provided for detecting and identifying DNase I nuclease hypersensitive sites in individual cells, comprising:
1) lysing the cells and digesting cellular DNA with DNase I;
2) ligating of chromatin DNA to a first barcode adaptor;
3) pooling of the nuclei followed by dilution and redistribution into separate plate well;
4) subjecting the DNA to reverse cross-linking, introducing a second barcode complementary to the first barcode adaptor via an amplification assay;
5) pooling of amplified DNA, ligating of the DNA to a second barcode adaptor;
6) amplifying the DNA and introducing a third barcode adaptor; and,
7) pooling and sequencing of amplified DNA wherein, the amplified DNA sequences having the same combination of barcodes are derived from a single cell; thereby, detecting and identifying nuclease hypersensitive sites in individual cells. In certain embodiments, the first barcode adaptor may be ligated to the chromatin DNA by Terminal Deoxynucleotidyl Transferase (TdT) and T4 ligase.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “amplify”, “amplification”, “amplification reaction”, or “amplifying” refer to any in vitro process for multiplying the copies of a target nucleic acid. Amplification sometimes refers to an “exponential” increase in target nucleic acid. However, “amplifying” may also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “illness” refers to any disease or condition afflicting a mammal such as a human, including for example, cancers, immune dysregulations, infections, neurological conditions, and genetic disorders.

The term “sample” in the present specification and claims is used in its broadest sense and can be, by non-limiting example, includes specimens or cultures (e.g., microbiological cultures), biological as well as non-biological specimens. Biological samples may comprise animal-derived materials, including fluid (e.g., blood, saliva, urine, lymph, etc.), solid (e.g. stool) or tissue (e.g., buccal, organ-specific, skin, etc.), as well as liquid and solid food and feed products and ingredients such as dairy items, vegetables, meat and meat by-products, and waste.

Biological samples may be obtained from, e.g., humans, any domestic or wild animals, plants, bacteria or other microorganisms, etc. These examples are not to be construed as limiting the sample types applicable to the present disclosure. Those of skill in the art would appreciate and understand the particular type of sample required for the detection of particular target sequences (Pawliszyn, J., Sampling and Sample Preparation for Field and Laboratory, (2002). Venkatesh Iyengar. G., et al., Element Analysis of Biological Samples: Principles and Practices (1998). Drielak .S., Hot Zone Forensics: Chemical, Biological. and Radiological Evidence Collection (2004); and Nielsen. D. M., Practical Handbook of Environmental Site Characterization and Ground-Water Monitoring (2005)).

As referred to herein, a “subpopulation” of cells refers to a particular subset of cells of a particular cell type which can be distinguished or are uniquely identifiable and set apart from other cells of this cell type. The cell subpopulation may be phenotypically characterized, and is preferably characterized by methods embodied herein. A cell (sub)population as referred to herein may constitute of a (sub)population of cells of a particular cell type characterized by a specific cell state.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Any compositions or methods provided herein can be combined with one or more of any of the other compositions and methods provided herein.

All publications, published patent documents, and patent applications cited herein are hereby incorporated by reference to the same extent as though each individual publication, published patent document, or patent application was specifically and individually indicated as being incorporated by reference.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1J are a series of plots demonstrating the co-profiling H3K4me3 or RNAPII and RNA at single cell levels. FIG. 1A. A genome browser snapshot showing six panels of data. From the top to the bottom, the first panel in blue shows the H3K4me3 profile of pooled (3,717) single cells from the joint measurement of H3K4me3 and RNA using the scPCOR-seq assay. The second panel in red shows the bulk cell H3K4me3 profile of ENCODE ChIP-seq data for 293T cells. The third panel in green shows the bulk cell H3K4me3 profile of ENCODE ChIP-seq data for H1 ES cells. The fourth panel in yellow shows the bulk cell H3K4me3 profile of ENCODE ChIP-seq data for GM12878 cells. The fifth panel in blue shows the RNA profile of pooled (3,713) single cells from the joint measurement of H3K4me3 and RNA using the scPCOR-seq assay. The sixth panel in red shows the bulk cell RNA-seq profile for 293T cells. The seventh panel in green shows the bulk cell RNA-seq profile for H1 ES cells. The eighth panel in green shows the bulk cell RNA-seq profile for GM12878 cells. FIG. 1B. A scatter plot showing the correlation between the H3K4me3 peaks detected from the ENCODE bulk 293T cell ChIP-seq data and that from the pooled single cell H3K4me3 data from scPCOR-seq assay. FIG. 1C. A scatter plot showing the correlation between the bulk 293T cell RNA-seq data and the pooled single cell RNA data from the scPCOR-seq assay. FIG. 1D. A plot showing the fraction of H3K4me3 reads in peaks versus the number of peaks detected per single cell from the scH3K4me3-scRNA measurement by scPCOR-seq. FIG. 1E. A genome browser snapshot showing six panels of data. From the top to the bottom, the first panel in blue shows the RNAPII profile of pooled (2347) single cells from the joint measurement of RNAPII and RNA using the scPCOR-seq assay. The second panel in red shows the bulk cell RNAPII profile of ENCODE ChIP-seq data for 293T cells. The third panel in green shows the bulk cell RNAPII profile of ENCODE ChIP-seq data for HI cells. The fourth panel in blue shows the RNA profile of pooled (2347) single cells from the joint measurement of RNAPII and RNA using the scPCOR-seq assay. The fifth panel in red shows the bulk cell RNA-seq profile for 293T cells. The sixth panel in green shows the bulk cell RNA-seq profile for H1 ES cells. FIG. 1F. A scatter plot showing the correlation between the RNAPII peaks detected from the ENCODE bulk H1 ES cell ChIP-seq data and that from the pooled single cell RNAPII data from scPCOR-seq assay. FIG. 1G. A scatter plot showing the correlation between the bulk H1 cell RNA-seq data and the pooled single cell RNA data from the scPCOR-seq assay. FIG. 1H. A plot showing the fraction of RNAPII reads in peaks versus the number of peaks detected per single cell from the scRNAPII-scRNA measurement by scPCOR-seq. FIG. 1I. A schematic diagram showed the experimental steps of scPCOR-seq. FIG. 1J. Two scatter plots showing the number of reads that mapped to human and mouse genome. left) for RNA reads. right) for H3K4me3 reads.

FIGS. 2A-2F are a series of plots and heat maps showing the clustering of single cells using either RNA-H3K4me3 or RNA-RNAPII scPCOR-seq data. FIG. 2A. A t-Distributed Stochastic Neighbor Embedding (t-SNE) plot showing the clusters of single cells using the RNA data from the RNA-H3K4me3 scPCOR-seq assay. A consensus clustering approach was applied to the RNA and H3K4me3 data from the scPCOR-seq RNA-H3K4me3 measurement. Single cells were clustered into two groups (Clus 1 in blue, Clus 2 in red, and Clus3 in orange). t-SNE was applied to the RNA data from the RNA-H3K4me3 measurement directly. The position of a single cell was determined by the two t-SNE components while the color was determined by the clusters obtained from the consensus clustering. FIG. 2B. A t-SNE plot showing the clustering of single cells using the H3K4me3 data from the RNA-H3K4me3 scPCOR-seq assay. A consensus clustering approach was applied to the RNA and H3K4me3 data from scPCOR-seq RNA-H3K4me3 measurement. Single cells were clustered into two groups (Clus 1 in blue, Clus 2 in red, and Clus3 in orange). t-SNE was applied to the H3K4me3 data from the RNA-H3K4me3 measurement directly. The position of a single cell was determined by the two t-SNE components while the color was determined by the clusters obtained from the consensus clustering. FIG. 2C. Annotation of cell clusters by overlap with cell-specific genes or H3K4me3 peaks. Top panel: A heatmap showing the overlap between the differential genes from different groups. Single cells were clustered into two groups in FIG. 2a. The differentially expressed genes between cluster 1, cluster 2, and cluster 3 were denoted as “Clus 1”, “Clus 2” and “Clus 3” as shown in the labels on the y-axis. The differentially expressed genes between H1, GM12878, and 293T cells were denoted as “H1”, “GM12878” and “293T” as shown in the labels on the x-axis. The significance of overlap is determined by the hypergeometric test, which is shown by the color level (negative log of the p-value). Bottom panel: Similar to the top panel but it is for the differential H3K4me3 peaks from different groups. The groups of H3K4me3 peaks are similar to those obtained for the top panel. FIG. 2D. A t-SNE plot showing the clusters of single cells using the RNA data from the RNA-RNAPII scPCOR-seq assay. The data were treated similarly as described in FIG. 2A. FIG. 2E. A t-SNE plot showing the clusters of single cells using the

RNAPII binding data from the RNA-RNAPII scPCOR-seq assay. The data were treated similarly as described in FIG. 2A. FIG. 2F. Annotation of cell clusters by overlap with cell-specific genes or RNAPII peaks. The data were treated similarly as described in FIG. 2C.

FIGS. 3A-3F are a series of plots and heat maps demonstrating the heterogeneity in gene expression and RNAPII bindings. FIG. 3A. Four scatter plots between two variables at the cell type specific genes. (top left) 293T mRNA CV vs. 293T RNAPII CV; (top right) 293T mRNA CV vs. H1 RNAPII CV; (bottom left) H1 mRNA CV vs. 293T RNAPII CV; (bottom right) H1 mRNA CV vs. H1 RNAPII CV. Each dot represents one cell-specific gene. FIG. 3B. The cell-to-cell variation is negatively correlated to RNA and RNAPII density. The heatmap shows the correlation coefficient between two variables at the cell type specific genes. Totally there are eight variables including mRNA density in H1 cells, RNAPII density in H1 cells, mRNA density in 293T cells, RNAPII density in 293T cells, mRNA cell-to-cell variation in H1 cells, RNAPII cell-to-cell variation in H1 cells, mRNA cell-to-cell variation in 293T cells, RNAPII cell-to-cell variation in 293T cells. This negative correlation is specific to both assay and cell type. FIG. 3C. RNAPII bound to different regions displays different cell-to-cell variation in HI cells. Genes were separated to three groups based on the location where RNAPII binding was detected: (1) in the TSS region (+/−2 kb surrounding TSS), (2) in the gene body region, and (3) in the TES regions (+/−2 kb surrounding TES). The cell-to-cell variation in RNAPII binding is plotted for each groups of genes. The P-value is computed by Wilcoxon's rank sum test. FIG. 3D. RNAPII bound to different regions displays different cell-to-cell variation in H1 cells. Similar to Panel c but for 293T cells. FIG. 3E. Genes with RNAPII bound to different regions display different cell-to-cell variation in expression in H1 cells. The cell-to-cell variation in expression in H1 cells for each group of genes identified in Panel c is plotted. The P-value is computed by Wilcoxon's rank sum test. FIG. 3F. Genes with RNAPII bound to different regions display different cell-to-cell variation in expression in 293T cells. Similar to Panel e but for 293T cells.

FIGS. 4A-4I are a series of schematics and plots demonstrating that the co-profiling of RNAPII and RNA by scPCOR-seq predicts cis regulatory elements. FIG. 4A. Identification of CRE-gene interaction by correlating RNAPII binding density at CREs and RNA level of genes. COL1A2 is an H1-specific gene while ALDHIA2 is a 293T-specific gene. The schematic diagram shows that there are more CRE-gene interactions in H1 cells than 293T cells at COLIA2 gene. Similarly, there are more CRE-gene interactions in 293T cells than H1 cells at ALDH1A2 gene. FIG. 4B. Significant CRE-gene interactions identified at COLIA2 (left) and ALDH1A2 (right) in H1 and 293T cells, respectively. FIG. 4C. Violin plots showing the averaged CRE-gene interaction strength for H1-specific genes in H1 cells and 293T cells. H1-specific genes were identified by comparing the ENCODE RNA-seq datasets between H1 and 293T cells. FIG. 4D. Violin plots showing the averaged CRE-gene interaction strength for 293T-specific genes in H1 cells and 293T cells. FIG. 4E. Violin plots showing the averaged CRE-gene interaction strength at H1-specific CREs in H1 cells and 293T cells. H1-specific CREs were identified by comparing the CRE-gene interaction pairs from H1 and 293T cells. FIG. 4F. Violin plots showing the averaged CRE-gene interaction strength at 293T-specific CREs in H1 cells and 293T cells. FIG. 4G. TrAC-looping data indicate physical interactions between CREs and genes. An example shows the identified PETs (paired-end tags) linking a CRE and gene pair. The PETs were visualized at the bottom. FIG. 4H. Violin plots showing the normalized H1 cell TrAC-looping PETs connecting the CRE and gene TSS regions for the H1-specific and 293T-specific CRE-gene pairs, respectively. FIG. 4I. Violin plots showing the normalized GM12878 cell TrAC-looping PETs connecting the CRE and gene TSS regions for the H1-specific and 293T-specific CRE-gene pairs, respectively.

FIG. 5 is a schematic diagram showing the procedures of scPCOR-seq.

FIGS. 6A and 6B are plots showing that RNAPII binding is positively correlated with gene expression levels. Genes were separated into four groups based on the RNAPII binding levels in the pooled single cells (x-axis). The y-axis shows the RNA expression level of each group.

FIG. 7 are plots showing the correlation between mRNA level and RNAPII density. Four scatter plots between two variables at the cell type specific genes. (top left) 293T mRNA level vs. 293T RNAPII density (top right) 293T mRNA level vs. H1 RNAPII density (bottom left) H1 mRNA level vs. 293T RNAPII density (bottom right) H1 mRNA level vs. H1 RNAPII density.

FIGS. 8A and 8B are a schematic representation of an embodiment of iscChlC-seq. FIG. 8A. Experimental flow. (1) Bulk cells were split into the first 96 well plate after antibody guided MNase cleavage and end repair. (2) Barcoded cells were pooled together and sorted into the second 96 well plate to introduce i7 index. (3) Cells were pooled together again from each plate and labelled with i5 index in PCR2. FIG. 8B. Illustration of poly dG addition to DNA ends by TdT, oligo dC adaptor ligation by T4 DNA ligase, and PCR-mediated barcoding process. Cell barcode (red) is designed into the oligo dC P7 adaptor in which 3′ ends are blocked to prevent non-template tailing by TdT. After reverse crosslinking, barcoded DNA fragments could be efficiently labeled with i7 index (purple) through annealing and PCR extension. The barcoded P5 adaptor is added to the other end of genomic DNA fragments by ligation and PCR2, which is used to amplify the library DNA for NGS sequencing.

FIGS. 9A-9D are plots demonstrating that iscChIC-seq is a highly specific and sensitive method to detect H3K4me3 profiles in human white blood cells. FIG. 9A is a genome browser snapshot showing panels of H3K4me3 profiles in human white blood cells. The top blue track shows the pooled single cell data from iscChIC-seq. The bottom track shows 500 randomly selected single cells. The middle tracks display the ENCODE bulk cell ChIP-seq data from different cells indicated on the left. FIG. 9B is a Venn diagram showing the overlap of the enriched regions of H3K4me3 profiles measured by ChIP-seq using bulk cells and by the pooled single cell data. FIG. 9C is a scatter plot of the H3K4me3 read density of ChIP-seq (bulk cell) versus that of pooled single cells from iscChlC-seq (2,000 cells) at the genome-wide divided bins (the size of bin is 5 kb). The Pearson correlation is equal to 0.89. FIG. 9D is a TSS profile plot showing the H3K4me3 profile around TSS for all single cells (grey) and the pooled single cells (red).

FIGS. 10A-10D are plots and a heatmap demonstrating the identification of sub-cell types in white blood cells based on clusters generated from single-cell H3K4me3 profiles. FIG. 10A is a t-SNE visualization of cells by applying the t-SNE analysis on the consensus matrix. Cell type annotations of clusters are obtained by the analysis in FIG. 10B. FIG. 10B is a heatmap showing the significance of the overlap between the cluster-specific peaks from the H3K4me3 iscChIC-seq data (FIG. 10A) and cell type-specific peaks from ENCODE H3K4me3 ChIP-seq data. The Y-axis refers to the cluster-specific peaks and X-axis refer to the cell type-specific peaks. The values before the +/− sign refer to the average negative logarithm of the P-value for the overlap between the two types of peaks over 100 subsamples. The values behind the +/− sign refer to the standard deviation of the negative logarithm of the P-value over 100 sub samples. FIG. 10C is a series of genome browser snapshots showing the H3K4me3 profiles from bulk cells ChIP-Seq data and pooled single-cell iscChlC-seq data. The ChIP-Seq data for B cells, monocytes, T cells and, NK cells are downloaded from ENCODE (red). The pooled H3K4me3 iscChIC-seq data for each identified cell type (FIG. 10A) are displayed (blue). FIG. 10D is a t-SNE visualization of cells by applying the t-SNE analysis on the consensus matrix. H3K4me3 density of regions associated with different genes is plotted. The color level indicates the H3K4me3 density level.

FIGS. 11A-11E are a series of plots, a genome browser and a Venn diagram demonstrating that iscChlC-seq is a highly specific and sensitive method to detect H3K27me3 profiles in human white blood cells. FIG. 11A is a genome browser snapshot showing H3K27me3 profiles in human white blood cells. The top blue track shows the pooled single cell data from iscChlC-seq. The bottom track shows 500 randomly selected single cells. The middle tracks display the ENCODE bulk cell ChIP-seq data from different cells indicated on the left. FIG. 11B is a Venn diagram showing the overlap of the enriched regions of H3K27me3 profiles measured by ChIP-seq using bulk cells and by the pooled single cell data. FIG. 11C is a scatter plot of the H3K27me3 read density of ChIP-seq (bulk cell) versus that of pooled single cells from iscChlC-seq (2,000 cells) at the genome-wide divided bins (the size of bin is 50 kb). The Pearson correlation is equal to 0.92. FIG. 11D is a t-SNE visualization of cells by applying the t-SNE analysis on the consensus matrix. Cell type annotations of clusters are obtained by the analysis in FIG. 11E. FIG. 11E is a heatmap showing the significance of the overlap between the cluster-specific peaks from the H3K27me3 iscChlC-seq data (FIG. 4D) and cell type-specific peaks from ENCODE H3K27me3 ChIP-seq data. The Y-axis refers to the cluster-specific peaks and X-axis refer to the cell type-specific peaks. The values before the +/− sign refer to the average negative logarithm of the P-value for the overlap between the two types of peaks over 100 subsamples. The values behind the +/− sign refer to the standard deviation of the negative logarithm of the P-value over 100 sub samples.

FIGS. 12A-12C are a series of graphs and plots demonstrating the correlation of cell clusters revealed from the single cell H3K4me3 and H3K27me3 data by bivalent domains. FIG. 12A. The cluster-specific peaks identified from the single-cell H3K4me3 and H3K27me3 data exhibit the highest overlap if they are from the same cell type. For each subplot, the cluster-specific peaks of H3K4me3 from one annotated cluster (as indicated on the top) were compared with the cluster-specific peaks of H3K27me3 from different clusters (as indicated below the plot). The Y-axis in each subplot indicates the −log 2 of P-value for the overlap between the cluster-specific peaks of H3K4me3 and cluster-specific peaks of H3K27me3. FIG. 12B is a scatter plot between the cell-to-cell variation of H3K4me3 and H3K27me3 for clusters annotated as monocytes in bivalent domains. FIG. 12C. Cluster-specific bivalent domains associated with H3K4me3 and H3K27me3 were computed for the purpose of finding the relationship between cell-to-cell variation in H3K4me3 and H3K27me3. For each comparison between the H3K4me3 and H3K27me3 clusters, the overlap between cluster-specific bivalent domains was considered, the Spearman correlation between the coefficient of variation in H3K4me3 and H3K27me3 for these selected bivalent domains was calculated.

FIGS. 13A and 13B are a series of plots, heatmaps and a genome browser snapshot showing the pooled H3K4me3 iscChIC-seq profiles for series of cell percentages. FIG. 13A is a genome browser snapshot showing tracks of aggregated H3K4me3 iscChlC-seq signals from different percentages of cells. The genomic region is same to that of FIG. 9A. Cells were sorted by descending number of unique reads per cell. FIG. 13B are TSS profile plots and heatmaps showing aggregated iscChIC-seq signals around TSS from different percentages of cells. The plots were generated by deeptools (Ramirez F. et al. 2016. deepTools2: a next generation web server for deep-sequencing data analysis. Nucleic Acids Research 44: W160-W165).

FIGS. 14A-14D demonstrate a clustering analysis using the single cell H3K4me3 and H3K27me3 data. FIG. 14A. The clustering method was applied to the single cell H3K4me3 data with varying the number of clusters. In each cluster, its silhouette value was plotted in the y-axis. FIG. 14B. The frequency of having significant annotation of H3K4me3 clusters was plotted. FIG. 14C. The clustering method was applied to the single cell H3K27me3 data with varying the number of clusters. In each cluster, its silhouette value was plotted in the y-axis. FIG. 14D. The frequency of having significant annotation of H3K27me3 clusters was plotted.

FIG. 15 shows that for each subplot (subplots for top left, top right, bottom left, bottom right are for cluster annotated to B, Mono, T, and NK, respectively), peaks were identified for the H3K4me3 pooled cells from a cluster and compared with the cell type specific peaks identified from H3K4me3 ENCODE data. The Y-axis is the fraction of the cell type specific peaks recovered by the peaks identified from pooled single cell data.

FIGS. 16A-16D show a comparison of gene expression for genes related to the cell-type-specific peaks that were recovered in FIG. 15. FIG. 16A. Genes closely related to the recovered H3K4me3 B cell specific peaks by pooled single cells were identified. The gene expression of this set of genes were examined in B, Mono, T, and NK cells. The P-value between the gene expression of different cell types were computed using Wilcoxon's ranksum test. FIG. 16B. Similar to FIG. 16A, but for the recovered H3K4me3 Mono specific peaks. FIG. 16C. Similar to FIG. 16A, but for the recovered H3K4me3 T specific peaks. FIG. 16D. Similar to FIG. 16A, but for the recovered H3K4me3 NK specific peaks.

FIGS. 17A and 17B. Pooled H3K27me3 iscChlC-seq profiles for series of cell percentages. FIG. 17A is a genome browser snapshot showing tracks of aggregated H3K27me3 iscChlC-seq signals from different percentages of cells. The genomic region is same to that of FIG. 16A. Cells were sorted by descending number of unique reads per cell. FIG. 17B is a series of TSS profile plots and heatmaps showing aggregated iscChlC-seq signals around TSS from different percentages of cells.

FIGS. 18A-18D are a series of plots, a Venn diagram and a genome browser snapshot demonstrating that iscDNase-seq detects open chromatin regions in single cells. FIG. 18A is a genome browser snapshot showing chromatin accessibility detected by the pooled iscDNase-seq data and ENCODE bulk cell DNase-seq data for different immune cell types. The top track referred to the pooled iscDNase-seq data for human white blood cells. The other tracks, from the top to the bottom, referred to the ENCODE bulk cell DNase-seq data for Th1, Th2, Treg, B cells, monocytes, and NK cells, respectively. FIG. 18B is a Venn diagram showing the overlap between the DHSs obtained from the ENCODE DNase-seq data and the pooled single cell DNase-seq data. FIG. 18C is a scatter plot showing the correlation between the read density of the bulk cell DNase-seq and pooled single cell DNase-seq at the DHSs. The correlation was computed using Pearson Correlation. FIG. 18D is a TSS plot showing the TSS enrichment score of the pooled iscDNase-seq data.

FIGS. 19A-19F are a series of plots and heatmaps demonstrating that iscDNase-seq detects different sub cell types in human white blood cells and their specific regulatory regions. FIG. 19A shows a t-SNE visualization of cells with annotation of cells using the cluster information. FIG. 19B shows a t-SNE visualization of cells using the cell type information including the human WBCs, sorted B cells, sorted T cells, sorted NK cells, and sorted monocytes. FIG. 19C is a bar plot showing the accuracy of cell clusters. FIG. 19D shows a t-SNE visualization of cells with the accessibility of selected TF genes. The color level indicates the zscore of accessibility across all the cells. Four TF genes were selected including (top left) PAX5, (top right) CEBPB, (bottom left) TCF7, and (bottom right) MAF. FIG. 19E is a heatmap demonstrating that the cluster-specific peaks show distinct enrichment in different cell types. A heatmap showing the z-score of the normalized read count at the specific peaks for each cluster. FIG. 19F is a heatmap showing key transcription factor motifs enriched in the cluster-specific DHS peaks. Motif enrichment analysis was performed for each group of top specific peaks. The 80 most significant motifs were selected for each cluster. We eliminated those motifs that existed in more the one cluster. A heatmap was shown for the -log (P-value) for these TF motifs in each cluster.

FIGS. 20A-20G are a series of plots, Venn diagrams and a genome browser track demonstrating that iscDNase-seq predicts functional open chromatin regions. FIG. 20A is a bar plot showing the overlap between the cell type specific peaks from dscATAC-seq and the cell type specific peaks from the iscDNase-seq. Each subplot refers to the comparison between the cell type specific peaks from dscATAC-seq in one cell type with the cell type specific peaks from iscDNase-seq in four cell types. FIG. 20B is a series of Venn diagrams showing the overlap between peak sets from bulk DNase-seq and bulk ATAC-seq in B cells (left) and the overlap between the peak sets from iscDNase-seq and dscATAC-seq in B cells (right). FIG. 20C is a Genome Browser track showing similarities and differences between the iscDNase-seq and dscATAC-seq datasets at the PAX5 gene locus in B cells. FIG. 20D is a violin plot showing the fraction of nucleotides (A,T,C and G) at the unique peaks from iscDNase-seq and dscATAC-seq for B cells. FIG. 20E is a violin plot showing the fraction of nucleotides (A,T,C and G) at the unique peaks from bulk cell DNase-seq and bulk cell ATAC-seq for B cells. FIG. 20F is a plot showing sequence conservation scores from B cells for the unique iscDNaseq peaks and unique dscATAC-seq peaks. The unique peaks detected by iscDNase-seq are more likely conserved peaks than those uniquely detected by dscATAC-seq. FIG. 20G is a violin plot showing the gene expression levels in B cells of genes associated with unique iscDNase-seq, unique dscATAC-seq peaks.

FIGS. 21A-21G are a series of plots and schematic diagrams showing the cell-to-cell variation in DHS detected by iscDNase-seq is highly correlated with variation in gene expression. FIG. 21A is a schematic diagram showing the calculation for the correlation between cell-to-cell variation in gene expression and accessibility. First, Genes are annotated to the nearest DHSs located within the selected genomic regions enclosed by the red brackets. Second, we computed the density table and gene expression table for dscATAC-seq/iscDNase-seq and scRNA-seq, respectively. Also, for each gene and DHSs, we computed the coefficient of variation. Third, more than one DHS may be annotated to a gene. If it was the case, an average coefficient of variation (CV) was taken over DHSs which were annotated to the same gene. Forth, 20 genes were grouped in a group based on their CV in accessibility. Fifth, we computed the averaged CV for each group of genes and each assay. Spearman correlation was computed between CV obtained from scRNA-seq and iscDNase-seq/dscATAC-seq over the groups of genes.

FIG. 21B. By varying the selection of the genomic regions enclosed by the red brackets, multiple correlation coefficients are obtained. In particular, the DHS regions closest to the TSSs were first selected. Then the DHS regions with increasing distance from the TSSs were selected. FIG. 21C. The correlation between cell-to-cell variation in gene expression and accessibility for T cells were plotted as a function of distance, in which distance refers to the distance between the selected genomics regions and the closest TSSs. Correlation for both dscATAC-seq (red) and iscDNase-seq (blue) were computed. FIG. 21D. A violin plot for correlation between cell-to-cell variation in gene expression and accessibility for B cells for both dscATAC-seq and iscDNase-seq were plotted. FIG. 21E. A violin plot for correlation between cell-to-cell variation in gene expression and accessibility for monocytes for both dscATAC-seq and iscDNase-seq were plotted. FIG. 21F. A violin plot for correlation between cell-to-cell variation in gene expression and accessibility for T cells for both dscATAC-seq and iscDNase-seq were plotted. FIG. 21G. A violin plot for correlation between cell-to-cell variation in gene expression and accessibility for NKcells for both dscATAC-seq and iscDNase-seq were plotted.

FIG. 22 is a schematic illustration of iscDNase-seq methods. Experimental flow chart of the iscDNase-seq protocol.

FIG. 23 is a schematic illustration of TdT and T4 Ligation strategy. The sequence of reaction is as following: (1) addition of several dGs to the 3′ end of DNA by TdT; (2) annealing of oligo-dC barcode primer to the oligo dG sequence; (3) repairing the oligo-dG and T7 adaptor sequences by T4 DNA ligase.

FIGS. 24A-24C are plots demonstrating the quality control of the iscDNase-seq. FIG. 24A. A knee plot for the iscDNase-seq single cell data. FIG. 24B. A distribution plot for the reads per cell in which reads is in the log 10 scale. FIG. 24C. Human and mouse cells were mixed before the DNase I digestion step. Following the library construction and sequencing, the normalized numbers of sequence reads mapped to either the human (y-axis) and mouse (x-axis) genomes from each single cell were plotted. Each dot represents one barcodes. The number of reads were normalized by the total number of reads in the well.

FIGS. 25A and 25B are plots graph demonstrating the sequencing depth in each cell and TF Motifs enriched in clusters. FIG. 25A. A t-SNE visualization of cells with the number of non-duplicated reads. FIG. 25B. Bar plot showing the gene expression (rpkm) in monocytes, T cells, B cells, and NK cells for selected TFs. IRF8, CEBPA, TCF7, MAG were selected.

FIGS. 26A-26C are a series of Venn diagrams between iscDNase-seq and dscATAC-seq for T cells, NK cells and monocytes (right). Venn diagrams between bulk cell DNase-seq and ATAC-seq for T cells, NK cells and monocytes (left).

FIGS. 27A-27D are a series of heatmaps showing a gene ontology analysis for the unique iscDNase-seq peaks and unique dscATAC-seq peaks. The four heatmaps are for (FIG. 27A) B cells, (FIG. 27B) monocytes, (FIG. 27C) T cells, and (FIG. 27D) NK cells.

FIG. 28 is a series of violin plots showing the fraction of nucleotides (A, T, C, and G) for iscDNase-seq and dscATAC-seq (left). Violin plots showing the fraction of nucleotides (A, T, C, and G) for bulk cell DNase-seq and bulk cell ATAC-seq (right).

FIGS. 29A-29C are a series of sequence conservation score plots for unique iscDNase-seq and unique dscATAC-seq peaks for (FIG. 29A) Monocytes, (FIG. 29B) T cells, and (FIG. 29C) NK cells.

FIGS. 30A-30C are a series of violin plots showing the gene expression levels for genes associated with the unique iscDNase-seq peaks and unique dscATAC-seq peaks for (FIG. 30A) Monocytes, (FIG. 30B) T cells, and (FIG. 30C) NK cells.

FIGS. 31A-31D are a series of violin and UMAP plots and a heatmap demonstrating the co-profiling H3K4me3 and RNA at single cell level using H1, GM12878 and 293T cells. FIG. 31A. A violin plot showing measurement of four metrics for the RNA part of scPCOR-seq. The four metrics are Number of UMI, Number of useful UMI, Fraction of useful UMI, Number of genes detected. FIG. 31B. A violin plot showing measurement of four metrics for the H3K4me3 part of scPCOR-seq. The four metrics are Number of unique reads, Number of reads in peaks, Fraction of reads in peaks, Number of peaks detected. FIG. 31C. UMAP plots showing the clusters of single cells using the RNA data (left) and H3K4me3 (right) from the H3K4me3-RNA scPCOR-seq assay. A multilayer Louvain clustering was applied to jointly cluster single cells from both RNA and ChIC parts. FIG. 31D. A heatmap showing the overlap between the differential genes from different groups. Single cells were clustered into three groups in FIG. 2d. The differential expressed genes between cluster 1, cluster 2, and cluster 3 were denoted as “Clus 1”, “Clus 2” and “Clus 3” as shown in the labels on the y-axis. The differential expressed genes between the RNA-seq of 293T, GM12878 and H1 cells were denoted as “293T”, “GM12878” and “H1” as shown in the labels on the x-axis. The significance of overlap is determined by the hypergeometric test, which is shown by the color level (negative log of the p-value) (Right panel) Similar to the left panel, but it is for the differential H3K4me3 peaks from different groups. The groups are like those obtained from the left panel.

FIGS. 32A-32D are a series of violin plots, scatter plots, a heatmap and UMAP plots demonstrating the co-profiling PolII and RNA at single cell level using H1 and 293T cells. FIG. 32A. A violin plot showing measurement of four metrics for the RNA part of scPCOR-seq. The four metrics are Number of UMI, Number of useful UMI, Fraction of useful UMI, Number of genes detected. FIG. 32B. A violin plot showing measurement of four metrics for the PollI part of scPCOR-seq. The four metrics are Number of unique reads, Number of reads in peaks, Fraction of reads in peaks, Number of peaks detected. FIG. 32C. UMAP plots showing the clusters of single cells using the RNA data (left) and PolII (right) from the PolII-RNA scPCOR-seq assay. A multilayer Louvain clustering was applied to jointly cluster single cells from both RNA and ChIC parts. FIG. 32D. (Left panel) A heatmap showing the overlap between the differential genes from different groups. Single cells were clustered into two groups in FIG. 32C. The differential expressed genes between cluster 1, cluster 2 were denoted as “Clus 1” and “Clus 2 as shown in the labels on the y-axis. The differential expressed genes between the RNA-seq of H1, and 293T cells were denoted as “H1” and “293T” as shown in the labels on the x-axis. The significance of overlap is determined by the hypergeometric test, which is shown by the color level (negative log of the p-value) (Right panel) Similar to the left panel, but it is for the differential PolII peaks from different groups. The groups are like those obtained from the left panel.

FIGS. 33A-33F are a series of violin plots, UMAP plots and a genome browser snapshot showing the co-profiling H3K4me3 and RNA at single cell level using CD34 and CD36 cells. FIG. 33A. A genome browser snapshot showing four panels of data. From the top to the bottom, the first panel in blue shows the H3K4me3 profile of pooled single cells from the joint measurement of H3K4me3 and RNA using the scPCOR-seq assay. The second panel in red shows the bulk cell H3K4me3 profile of ChIP-seq data for CD36 cells. The third panel in blue shows the RNA profile of pooled single cells from the joint measurement of H3K4me3 and RNA using the scPCOR-seq assay. The fourth panel in red shows the bulk cell RNA-seq profile for CD36 cells. FIG. 33B. (Top panel) A plot of Gene body coverage using the RNA data from scPCOR-seq data. (Bottom panel) A plot of TSS enrichment profile for H3K4me3 data from scPCOR-seq data. FIG. 33C. (Top left) A violin plot showing the number of useful UMI of the RNA from scPCOR-seq. (Top right) A violin plot showing the number of genes recovered of the RNA from scPCOR-seq. (Bottom left) A violin plot showing the number of unique reads in peaks of the H3K4me3 from scPCOR-seq. (Bottom right) A violin plot showing the number of peaks of the H3K4me3 from scPCOR-seq. FIG. 33D. Two UMAP plots for scPCOR-seq that applied to H3K4me3 and RNA in CD34 and CD36 cells. (Top) UMAP using RNA and (Bottom) UMAP using H3K4me3. FIG. 33E. The gene expression level of HBB and ILIR2 are shown in the UMAP plots from mRNA data in the top left and top right plots, respectively. H3K4me3 density of HBB and ILIR2 are shown in the UMAP plots from H3K4me3 data in the bottom left and bottom right plots, respectively. FIG. 33F. (Upper panel) A violin plot showing the expression of the genes, which are different between the Day 5A group and Day 5B group cells, in CD36 Day-2 cells, CD36 Day-5A cells, and CD36 Day-5B cells. (lower panel) A violin plot showing the H3K4me3 density for genes in the top panel in CD36 Day-2 cells, CD36 Day-5A cells, and CD36 Day-5B cells.

### DETAILED DESCRIPTION

The disclosure provides a novel technique, termed scPCORseq herein (single-cell Profiling of Chromatin Occupancy and RNAs Sequencing), for simultaneously profiling genome-wide chromatin protein binding or histone modification marks and RNA expression in the same cell. It was demonstrated, as described in detail in the examples section which follows, that scPCOR-seq is able to profile either histone H3 lysine 4 trimethylation (H3K4me3) or RNA Polymerase II (RNAPII) and RNAs in a mixture of human H1, GM12878 and 293T cells at a single-cell resolution and either H3K4me3, RNAPII, or RNA profile can correctly separate the cells. It was observed that the cell-to-cell variation in RNAPII binding is dependent on its genomic location and is correlated with the cell-to-cell variation in gene expression. It was demonstrated that not only does RNAPII binding to the transcription start site (TSS) regions, but also its binding to the transcription end sites (TES) regions, contributes to the cellular heterogeneity in gene expression. The data revealed thousands of CRE-gene interaction pairs from the single-cell RNAPII binding and RNA co-profiling data, which may contribute to the cell-to- cell variation in expression. Overall, the composition and methods embodied herein, provides a powerful and novel approach to understand the relationships among different omics layers.

Accordingly, in certain embodiments, a method for simultaneous profiling of chromatin occupancy and RNA in a single cell comprises isolating and culturing cells of interest from a sample; contacting the cells with a fixative agent; performing guided chromatin cleavage; subjecting the cells to reverse transcription; subjecting the cells to terminal deoxynucleotidyl transferase (TdT)-mediated oligonucleotides to both cDNA and chromatin cleaved ends in the presence of an oligonucleotide adaptor; pooling the cells from each reaction well and sorting the pooled cells, followed by one or more amplification steps; and, subjecting the sorted cells to a library sequencing; thereby, simultaneously profiling of chromatin occupancy and RNA in a single cell.

### Chromatin Immunocleavage

The basic idea of the chromatin immunocleavage (ChIC) method is to indirectly tether a nuclease, whose activity can be controlled, to antibodies that are specifically bound to a chromatin protein of interest. Subsequent activation of the tethered nuclease should result in DNA cleavage in the vicinity of the chromatin bound protein. Mapping of such DNA cleavage sites provides information about the genomic interaction sites of the protein of interest. In certain embodiments,

Micrococcal nuclease (MNase) is the enzyme of choice since its robust enzymatic activity stringently depends on Ca2+ions of millimolar (optimal at 10 mM) concentrations. This enzyme introduces DNA double-strand breaks in chromatin at nucleosomal linker regions and at nuclease hypersensitive (HS) sites.

To tether MNase to antibodies, a fusion protein consisting of two immunoglobulin binding domains of staphylococcal protein A that are N-terminally fused with MN are prepared. The protein (called pA-MNase) has a molecular weight of 34 kDa. In a general sense, the ChIC method is akin to the antibody-staining techniques for immunofluorescence studies, where the last step involves the addition of pA-MN. ChIC differs also from the staining techniques in that it is carried out in solution, where excess antibodies and pA-MN are removed by centrifugation in a microfuge.

### Single-Cell Barcode-Adaptors

The present disclosure also provides methods for labeling and identifying nucleic acid sequences using adaptors. An adaptor is an oligonucleotide composed of natural nucleotides, modified nucleotides, and/or synthetic (e.g., non-natural) nucleotides. An adaptor may be composed of DNA nucleotides, RNA nucleotides, RNA and DNA nucleotides (forming a RNA/DNA hybrid), synthetic nucleotides, modified nucleotides, and combinations of two or more of these. An adaptor may be in any conformation known in the art for oligonucleotides. Non-limiting examples of adaptor conformations include single-stranded, double-stranded, a mixture of single-stranded and double stranded, or hairpin-forming. The adaptor may be 15-100 nucleotides in length. In some embodiments, the adaptor is 15-45 nucleotides in length.

In some embodiments, an adaptor comprises a single-cell barcode (hereinafter referred to as “single-cell barcode-adaptors” or “barcode-adaptors”). A single-cell barcode is a sequence of nucleotides, typically up to 20 nucleotides but which can be longer, and is unique to each single cell. A single-cell barcode may be composed of DNA nucleotides, RNA nucleotides, RNA and DNA nucleotides (forming a RNA/DNA hybrid), synthetic nucleotides, modified nucleotides, and combinations of two or more of these. A single-cell barcode may be incorporated into the 5′ end of the adaptor. A single-cell barcode may be incorporated into the 3′ end of the adaptor. A single-cell barcode may be incorporated into the middle (e.g., not at the 5′ end or the 3′ end) of the adaptor.

In some embodiments, a single-cell barcode-adaptor oligonucleotide is “bead-bound,” i.e., is immobilized on a bead, or other solid object, that is modified to bind nucleotides. In some embodiments, a bead is a microsphere that binds single-cell barcode-adaptors. Beads can be individually assayed or isolated based on the physical characteristics of the bead. Beads for binding single-cell barcode-adaptors may be polystyrene beads, magnetic beads, hydrogel, or silica beads. In some embodiments, the 5′ end of the single-cell barcode-adaptor is bound to a bead and the 3′ end is not bound to a bead. In some embodiments, the 3′ end of the single-cell barcode-adaptor is bound to a bead and the 5′ end is not bound to a bead.

In other embodiments, a single-cell barcode-adaptor is not immobilized on a bead (i.e., neither end is bound to a bead), which is also referred to herein as being “free,” e.g., a “free single-cell barcode-adaptor.”

The single-cell barcode-adaptors may be single-stranded or double-stranded. In some embodiments, the single-cell barcode-adaptors are single-stranded.

In some embodiments, the adaptors contain a unique molecule identifier (UMI) sequence. In some embodiments, the single-cell barcode-adaptors contain a UMI. A UMI is a molecular tag of nucleotides that is used to detect and quantify unique RNA transcripts from a population as opposed to artifacts from PCR amplification. In some embodiments, the UMI sequence is random. A UMI sequence may be 4-30 nucleotides in length. In some embodiments, the UMI is 5-20 nucleotides in length. In some embodiments, the UMI is 6-12 nucleotides in length. In some embodiments, the UMI is 15-30 nucleotides in length.

In some embodiments, a plurality of single-cell barcode-adaptors molecules (e.g., bead-bound, free) are utilized. A plurality may include 2 or more single-cell barcode-adaptors molecules, 10 or more single-cell barcode-adaptors molecules, 100 or more single-cell barcode-adaptors molecules, 1,000 or more single-cell barcode-adaptors molecules, 10,000 or more single-cell barcode-adaptors molecules, 100,000 or more single-cell barcode-adaptors molecules, 1,000,000 or more single-cell barcode-adaptors molecules, or 10,000,000 or more single-cell barcode-adaptors molecules. In some embodiments, the plurality of single-cell barcode-adaptors molecules are utilized to sequence the RNA from a single cell. In some embodiments, the plurality of single-cell barcode-adaptors molecules are utilized to sequence the RNA from a plurality of cells.

In some embodiments, single-cell barcode-adaptors molecules (e.g., bead-bound, free) are blocked at or near the 3′ end of the adaptor. In some embodiments, single-cell barcode-adaptors molecules (e.g., bead-bound, free) are blocked at or near the 3′ end of the adaptor.

In certain embodiments, a plurality of single-cell barcode-adaptors molecules (e.g., bead-bound, free) may comprise the same nucleotide sequence or different nucleotide sequences. In some embodiments, the plurality of single-cell barcode-adaptors molecules comprise the same nucleotide sequence. In some embodiments, the plurality of single-cell barcode-adaptors molecules do not comprise the same nucleotide sequence. In some embodiments, the single-cell barcode-adaptors molecules comprise at least 2 different nucleotide sequences, at least 10 different nucleotide sequences, at least 100 different nucleotide sequences, at least 1,000 different nucleotide sequences, at least 10,000 different nucleotide sequences, at least 100,000 different nucleotide sequences, or any number of different nucleotide sequences between 2-100,000 different nucleotide sequences.

### Histone Modifications

Histone modifications, which are typically measured by chromatin immunoprecipitation (ChIP) with massively parallel DNA sequencing (Barski A., et al. 2007. High-resolution profiling of histone methylations in the human genome. Cell 129: 823-837; Johnson D S., et al. 2007. Genome-wide mapping of in vivo protein-DNA interactions. Science 316: 1497-1502; Mikkelsen T. S., et al. 2007. Genome-wide maps of chromatin state in pluripotent and lineage-committed cells. Nature 448: 553-560; Robertson G., et al. 2007. Genome-wide profiles of STAT1 DNA association using chromatin immunoprecipitation and massively parallel sequencing. Nature Methods 4: 651-657) at the bulk-cell level, are associated with transcriptional regulation. Chromatin regions enriched in 113K4 methylation and H3K27 acetylation are potentially active promoters or enhancers that activate the transcription of target genes; on the other hand, genes enriched in H3K27me3 signals are usually repressed (Kim T. H., et al. 2005. A high-resolution map of active promoters in the human genome. Nature 436: 876-880.2005; Barski A., et al. 2007; Mikkelsen T. S., et al .; Wei G. et al. 2009. Global mapping of H3K4me3 and H3K27me3 reveals specificity and plasticity in lineage fate determination of differentiating CD4⁺ T cells. Immunity 30: 155-167; Creyghton M. P., et al. 2010. Histone H3K27ac separates active from poised enhancers and predicts developmental state. Proc Natl Acad Sci U SA 107: 21931-21936). While the genomic profiles of various histone modifications have been extensively characterized at the bulk cell level, several single-cell epigenomic techniques for detecting histone modification marks are reported recently (Rotem A., et al. 2015. Single-cell ChIP-seq reveals cell subpopulations defined by chromatin state. Nat Biotechnol 33: 1165-1172; Ai S. et al. 2019. Profiling chromatin states using single-cell itChIP-seq. Nat Cell Biol 21: 1164-1172; Carter B. et al. 2019. Mapping histone modifications in low cell number and single cells using antibody-guided chromatin tagmentation (ACT-seq). Nat Commun 10: 3747; Grosselin K., et al. 2019. High-throughput single-cell ChIP-seq identifies heterogeneity of chromatin states in breast cancer. Nat Genet 51: 1060-1066; Hainer S. J., et al. 2019. Profiling of Pluripotency Factors in Single Cells and Early Embryos. Cell doi:10.1016/j.ce11.2019.03.014; Harada A., et al. 2019. A chromatin integration labelling method enables epigenomic profiling with lower input. Nat Cell Biol 21: 287-296; Kaya-Okur H. S., et al. 2019. CUT&Tag for efficient epigenomic profiling of small samples and single cells. Nature Communications 10; Ku W. L., et al. 2019. Single-cell chromatin immunocleavage sequencing (scChIC-seq) to profile histone modification. Nat Methods 16: 323-325; Wang Q. et al. 2019. CoBATCH for High-Throughput Single-Cell Epigenomic Profiling. Mol Cell 76: 206-216 e207).

Although single cell assays including scChIL-seq (Harada et al. 2019), scChlC-seq (Ku et al. 2019), uliCUT&RUN (Hainer et al. 2019), scCUT&Tag (Kaya-Okur et al. 2019), iACT-seq (Carter et al. 2019), CoBATCH (Wang et al. 2019), itChIP-seq (Ai et al. 2019) and scChlP-seq (Rotem et al. 2015; Grosselin et al. 2019) were developed recently for measuring histone marks, they have specific limitations. While scChIP-seq combined the droplet barcoding approach with ChIP-seq (Barski et al. 2007; Rotem et al. 2015; Grosselin et al. 2019), all other methods except for itChIP-seq replaced the traditional immunoprecipitation with antibody guided digestion of chromatin either via antibody-directed, transposase-mediated integration of a DNA tag and fragmentation (for scChIL-seq (Harada et al. 2019) and scCUT&Tag (Kaya-Okur et al. 2019), iACT-seq (Carter et al. 2019), CoBATCH (Wang et al. 2019)), or via DNA cleavage specifically around nucleosomes containing the target modification (Schmid M., et al. 2004. ChIC and ChEC; genomic mapping of chromatin proteins. Mol Cell 16: 147-157) (for uliCUT&RUN (Hainer et al., 2019) and scChlC-seq (Ku et al., 2019)). scChlP-seq (Rotem et al. 2015; Grosselin et al., 2019), with a relatively complicated workflow, could detect about 2000-4000 cells in one experiment with an average of 4000 reads per cell. Although iACT-seq, scCUT&Tag, uliCUT&RUN, itChIP-seq and scChIC-seq have simpler workflows and more cost-effective, iACT-seq and scCUT&Tag could detect an average of 2000-6000 reads per cells and the cell throughput of uliCUT&RUN, itChlP-seq and scChIC-seq is low. While scChIL-seq and CoBATCH worked well for detecting active marks, they were not optimal for detecting repressive marks in fixed samples considering the attenuated activity of Tn5 in non-accessible chromatin regions and its intrinsic bias towards open regions (Harada et al. 2019). Therefore, there is a need to develop a single cell technique for profiling histone marks with higher cell throughput, more widely applications and detection of more reads per cell.

Accordingly, a method of identifying and profiling histone modifications in individual cells comprises crosslinking cells with a cross-linking fixative agent; contacting the fixed cells with a chromatin specific guided nuclease for cleaving the chromatin; repairing of the nuclease cleaved ends by a polynucleotide kinase and adding of 5′-phosphates for poly nucleotide tailing and ligation; and, barcoding of the nuclease cleaved sites with a barcode adaptor and pooling of the cells; splitting of the cells and incubating the cells with a reverse cross-linking buffer; capturing of barcoded cellular DNA fragments and index labeling of the barcoded DNA fragments by a first amplification assay to produce DNA libraries; pooling and purifying the DNA libraries and poly A tailing the purified DNA libraries; ligating the poly A tailed to an adaptor and purifying the ligated DNA; performing a second amplification assay, isolating, purifying and sequencing the amplified fragments; thereby, identifying and profiling histone modifications in individual cells.

### Samples

Cells, nucleic acids and the like utilized in methods described herein may be obtained from any suitable biological specimen or sample, and often is isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female, and a subject may be any age (e.g., an embryo, a fetus, infant, child, adult).

A sample or test sample can be any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated. Fluid or tissue sample soften are collected in accordance with standard protocols hospitals or clinics generally follow. For blood, an appropriate amount of peripheral blood (e.g., between 3-40 milliliters) often is collected and can be stored according to standard procedures prior to or after preparation.

A sample or test sample can include samples containing spores, viruses, cells, nucleic acid from prokaryotes or eukaryotes, or any free nucleic acid. For example, a method described herein may be used for detecting nucleic acid on the outside of spores (e.g., without the need for lysis). A sample may be isolated from any material suspected of containing a target sequence, such as from a subject described above. In certain instances, a target sequence may be present in air, plant, soil, or other materials suspected of containing biological organisms.

Nucleic acid may be derived (e.g., isolated, extracted, purified) from one or more sources by methods known in the art. Any suitable method can be used for isolating, extracting and/or purifying nucleic acid from a biological sample, non-limiting examples of which include methods of DNA preparation in the art, and various commercially available reagents or kits, such as Qiagen's QIAamp Circulating Nucleic Acid Kit, QiaAmp DNAMini Kit or QiaAmp DNA Blood Mini Kit (Qiagen, Hilden, Germany), GENOMICPREP™, Blood DNA Isolation Kit (Promega, Madison, WI.), GFX™ Genomic Blood DNA Purification Kit (Amersham, Piscataway, N.J.), and the like or combinations thereof.

In some embodiments, a cell lysis procedure is performed. Cell lysis may be performed prior to initiation of an amplification reaction described herein (e.g., to release DNA and/or RNA from cells for amplification). Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract nucleic acids from the cells, followed by treatment with chaotropic salts. In some embodiments, cell lysis comprises use of detergents (e.g., ionic, nonionic, anionic, zwitterionic). In some embodiments, cell lysis comprises use of ionic detergents (e.g., sodium dodecyl sulfate (SDS), sodium lauryl sulfate (SLS), deoxycholate, cholate, sarkosyl) Physical methods such as freeze/thaw followed by grinding, the use of cell presses and the like also may be useful. High salt lysis procedures also may be used. For example, an alkaline lysis procedure may be utilized. The latter procedure traditionally incorporates the use of phenol-chloroform solutions, and an alternative phenol-chloroform-free procedure involving three solutions may be utilized. In the latter procedures, one solution can contain 15 mM Tris, pH 8.0; 10 mM EDTA and 100 μg/ml RNAse A; a second solution can contain 0.2N NaOH and 1% SDS; and a third solution can contain 3 M KOAc, pH 5.5, for example. In some embodiments, a cell lysis buffer is used in conjunction with the methods and components described herein.

Nucleic acid may be provided for conducting the methods embodied herein without processing of the sample(s) containing the nucleic acid. For example, in some embodiments, nucleic acid is provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment(e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%,97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%,83%, 84%, 85%, 86%, 87%, 88%, 89%, 90%, 91%, 92%, 93%, 94%, 95%, 96%,97%, 98%, 99% or greater than 99% free of other non-nucleic acid components.

### Amplification

An amplification process herein may be conducted over a certain length of time. In some embodiments, an amplification process is conducted until a detectable nucleic acid amplification product is generated. A nucleic acid amplification product may be detected by any suitable detection process and/or a detection process described herein. In some embodiments, an amplification process is conducted over a length of time within about 20 minutes or less. For example, an amplification process may be conducted within about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, an amplification process is conducted over a length of time within about 10 minutes or less.

Any suitable RNA or DNA amplification technique may be used. In certain embodiments, the RNA or DNA amplification is an isothermal amplification. In certain embodiments, the isothermal amplification comprises nucleic-acid sequence-based amplification (NASBA), recombinase polymerase amplification (RPA), loop-mediated isothermal amplification (LAMP), real-time loop-mediated isothermal amplification (RT-LAMP), strand displacement amplification (SDA), helicase-dependent amplification (HDA), or nicking enzyme amplification reaction (NEAR). In certain embodiments, non-isothermal amplification methods may be used which include, but are not limited to, PCR, multiple displacement amplification (MDA), rolling circle amplification (RCA), ligase chain reaction (LCR), ramification amplification method (RAM) cross-priming amplification (CPA) or smart amplification (SMAP).

The methods and components described herein may be used for multiplex amplification. Multiplex amplification generally refers to the amplification of more than one nucleic acid of interest (e.g., amplification or more than one target sequence). For example, multiplex amplification can refer to amplification of multiple sequences from the same sample or amplification of one of several sequences in a sample. Multiplex amplification also may refer to amplification of one or more sequences present in multiple samples either simultaneously or instep-wise fashion. For example, a multiplex amplification may be used for amplifying least two target sequences that are capable of being amplified (e.g., the amplification reaction comprises the appropriate primers and enzymes to amplify at least two target sequences). In some instances, an amplification reaction may be prepared to detect at least two target sequences, but only one of the target sequences may be present in the sample being tested, such that both sequences are capable of being amplified, but only one sequence is amplified. In some instances, where two target sequences are present, an amplification reaction may result in the amplification of both target sequences. A multiplex amplification reaction may result in the amplification of one, some, or all of the target sequences for which it comprises the appropriate primers and enzymes. In some instances, an amplification reaction may be prepared to detect two sequences with one pair of primers, where one sequence is a target sequence and one sequence is a control sequence (e.g., a synthetic sequence capable of being amplified by the same primers as the target sequence and having a different spacer base or sequence than the target). In some instances, an amplification reaction may be prepared to detect multiple sets of sequences with corresponding primer pairs, where each set includes a target sequence and a control sequence.

Accordingly, in certain embodiments the methods disclosed herein include amplification reagents. Polymerases are proteins capable of catalyzing the specific incorporation of nucleotides to extend a 3′ hydroxyl terminus of a primer molecule, such as, for example, an amplification primer, against a nucleic acid target sequence (e.g., to which a primer is annealed). Polymerases may include, for example, thermophilic or hyperthermophilic polymerases that can have activity at an elevated reaction temperature (e.g., above 55° C., above 60° C., above 65° C., above 70° C., above 75° C., above 80° C., above 85° C., above 90° C., above 95° C., above 100° C.). A hyperthermophilic polymerase may be referred to as a hyperthermophile polymerase. A polymerase having hyperthermophilic polymerase activity may be referred to as having hyperthermophile polymerase activity. A polymerase may or may not have strand displacement capabilities. In some embodiments, a polymerase can incorporate about 1 to about 50 nucleotides in a single synthesis. For example, a polymerase may incorporate about 5, 10, 15, 20, 25, 30, 35, 40, 45 or 50 nucleotides in a single synthesis. In some embodiments, a polymerase, can incorporate 20 to 40 nucleotides in a single synthesis. In some embodiments, a polymerase, can incorporate up to 50 nucleotides in a single synthesis. In some embodiments, a polymerase, can incorporate up to 40 nucleotides in a single synthesis. In some embodiments, a polymerase, can incorporate up to 30 nucleotides in a single synthesis. In some embodiments, a polymerase, can incorporate up to 20 nucleotides in a single synthesis.

In some embodiments, amplification reaction components comprise one or more DNA polymerases. In some embodiments, amplification reaction components comprise one or more DNA polymerases comprising: 9° N DNA polymerase; 9° Nm™ DNA polymerase; THERMINATOR™ DNA Polymerase; THERMINATOR™ II DNA Polymerase; THERMINATOR™ III DNA Polymerase; THERMINATOR™ gamma. DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; DEEP VENTR™ (exo-) DNA Polymerase; D DEEP VENTR™ DNA Polymerase; DYNAZYME™ EXT DNA; DyNAzyme™ II Hot Start DNA Polymerase; PHUSION™ High-Fidelity DNA Polymerase; VENTR® DNA Polymerase; VENTR® (exo-) DNA Polymerase; REPLIPHI™ Phi29 DNA polymerase; EquiPhi29 DNA polymerase; rBst DNA Polymerase, large fragment (ISOTHERM™ DNA polymerase); MASTERAMP™ AMPLITHERM™ DNA Polymerase; Tag DNA polymerase; Tth DNA polymerase; Tfl DNA polymerase; Tgo DNA polymerase; SP6 DNA polymerase; Tbr DNA polymerase; DNA polymerase Beta; and ThermoPhi DNA polymerase.

In some embodiments, amplification reaction components comprise one or more hyperthermophile DNA polymerases. Generally, hyperthermophile DNA polymerases are thermostable at high temperatures. For example, a hyperthermophile DNA polymerase may have a half-life of about 5 to 10hours at 95 degrees Celsius and a half-life of about 1 to 3 hours at 100 degrees Celsius. In some embodiments, amplification reaction components comprise one or more hyperthermophile DNA polymerases from Archaea. In some embodiments, amplification reaction components comprise one or more hyperthermophile DNA polymerases from Thermococcus. In some embodiments, amplification reaction components comprise one or more hyperthermophile DNA polymerases from Thermococcaceaen archaean. In some embodiments, amplification reaction components comprise one or more hyperthermophile DNA polymerases from Pyrococcus. In some embodiments, amplification reaction components comprise one or more hyperthermophile DNA polymerases from Methanococcaceae. In some embodiments, amplification reaction components comprise one or more hyperthermophile DNA polymerases from Methanococcus. In some embodiments, amplification reaction components comprise one or more hyperthermophile DNA polymerases from Thermus. In some embodiments, amplification reaction components comprise one or more hyperthermophile DNA polymerases from Thermus thermophiles.

### Methods of Use

Epigenetic modification of chromatin critically contributes to cancer development and subsequently epigenetic modification variations have been established as biomarkers for various cancers. During the past few years, accompanying the technical breakthroughs in single-cell RNA-seq technologies, scRNA-seq has been applied to multiple cancer samples, which discovered a broad range of cellular heterogeneity in cancer samples. Further studies have found that the cellular heterogeneity within the cancer samples critically impact the pathology of cancer and therapeutic decisions. Thus, the cellular heterogeneity information found within various cancers can serve as valuable biomarkers for diagnosis and treatment of cancers. Similar to the application of scRNA-seq technology to cancer samples, the scPCOR-seq technique can be applied to various cancers to discover both gene expression and epigenetic biomarkers of disease.

Other methods of use include virus infections, e.g. SARS-COV-2, such as pandemic COVID 19. COVID-19 is known to be lethal to some individuals but not to others and the lethality may be associated with uncontrolled over immune reaction of the individuals to the viral infection. High levels of interferon gamma gene activation is a critical component of the immune reaction. Gene regulation (activation and repression) is prepared by its epigenetic modification. Thus scPCOR-seq can be applied to individuals to screen for epigenetic variations in interferon gamma and other chemokine and cytokines genes, which may predict uncontrolled reaction upon COVID-19 development. This will serve as important biomarkers for therapeutic decisions. Other examples, include profiling blood samples of leukemia patients: diagnosis and therapeutic biomarkers; examining cellular heterogeneity of various solid tumor samples to accurately diagnose the stage and nature and disease; valuation of the heterogeneity and quality of CAR-T cells before infusion to the patient. This assay profiles both the transcriptome and epigenome of CAR-T cells and thus can provide comprehensive information on the cells. Blood stem cell therapy: provide profiles of white blood cells on both transcriptomes and epigenomes

In some aspects, the present disclosure provides methods of diagnosing a disease or disorder. Control samples may be from a known healthy subject or group of subjects (e.g., not having a disease or disorder), from a subject or group of subjects known to have a disease or disorder, or from a reference sequence, wherein the reference sequence is known to be associated with a disease or disorder. Non-limiting of diseases or disorders that may be diagnosed using methods of the present disclosure include cancer (e.g., brain cancers, lymphomas, leukemias, lung cancer, pancreatic cancer, breast cancer, renal cancer, prostate cancer, hepatic cancer, gastric cancer, bone cancer), autoimmune disorders (e.g., rheumatoid arthritis, lupus, Celiac disease, Sjögren's syndrome), and diabetes.

In some aspects, the methods embodied herein are used to identify different cell types. Non-limiting examples of cell types that may be identified with methods of the instant disclosure include tumors (e.g., solid tumors, serous tumors, brain tumors, spinal cord tumors, meninges tumors, lymphomas, pancreatic tumors, hepatic tumors, breast tumors, renal tumors, lung tumors, gastric tumors, colon tumors, bone tumors, leukemias), T cells (e.g., CD4.sup.+, CD8.sup.+, regulatory, helper), B cells (e.g., plasma cells, lymphoplasmacytoid cells, memory B cells, B-2 cells, B-1 cells), natural killer cells, stem cells (e.g., hematopoietic).

In some aspects, the methods embodied herein are used to identify the differentiation state of cells. Non-limiting examples of differentiation states that may be identified with methods of the instant disclosure include pluripotent (e.g., embryonic stem cells, induced stem cells), partially differentiated (e.g., hematopoietic stem cells), or terminally differentiated (e.g., neurons, myocytes, osteoblasts, glial cells, epithelial cells).

In some aspects, the methods embodied herein are used for a systematic analysis of genomic interactions between cells.

In some aspects, the methods embodied herein are used for combinatorial probing of cellular circuits, for dissecting cellular circuitry, for delineating molecular pathways, and/or for identifying relevant targets for therapeutics development.

In some aspects, the methods embodied herein are used to analyzing genetic signatures of cells (e.g. the composition of a solid tumor), such as molecular profiling at the single cell or cell (sub)population level.

In further related aspects, the disclosure relates to diagnostic (including monitoring the status of a subject), prognostic (including monitoring treatment efficacy), prophylactic, or therapeutic methods. Diagnostic or prognostic methods may comprise detecting the gene signatures, protein signature, and/or other genetic or epigenetic signature as discussed herein. Therapeutic or prophylactic methods according to the invention in particular may comprise modulating the responder phenotype, and may include modulating the gene signature, protein signature, and/or other genetic or epigenetic signature of cells or cell (sub)populations. Such methods include both in vitro as well as in vivo modulation.

As used herein, the term “gene signature” may be used interchangeably with the term “signature gene”. These terms relate to one or more gene (or one or more particular splice variants thereof), the (increased) expression or activity of which or alternatively the decreased or absence of expression or activity of which is characteristic for a particular (multi)cellular phenotype, i.e. the occurrence of such particular (multi)cellular phenotype may be identified based on the presence or absence of such gene signature. The signature may thus be characteristic of a particular phenotype, but may also be characteristic of a particular immune cell subpopulation within a particular phenotype. Similarly, an “epigenetic signature” relates to one or more epigenetic element (or modification), the (increased) occurrence of which or alternatively the absence of which is characteristic for a particular (multi)cellular phenotype, i.e. the occurrence of such particular (multi)cellular phenotype may be identified based on the presence or absence of such epigenetic signature. As used herein a signature encompasses any gene or genes or epigenetic element(s) whose expression profile or whose occurrence is associated with a specific cell type, subtype, or cell state of a specific cell type or subtype within a population of cells. Increased or decreased expression or activity or prevalence may be compared between different phenotypes in order to characterize or identify specific phenotypes. A gene signature as used herein, may thus refer to any set of up- and down-regulated genes between two (multi)cellular states or phenotypes derived from a gene-expression profile. For example, a gene signature may comprise a list of genes differentially expressed in a distinction of interest; (e.g., high responders versus low responders; diseased state versus normal state; etc.). Similarly, an epigenetic signature as used herein, may thus refer to any set of induced or repressed epigenetic elements between two (multi)cellular states or phenotypes derived from an epigenetic profile. For example, an epigenetic signature may comprise a list of epigenetic elements differentially present in a distinction of interest; (e.g., high responders versus low responders; diseased state versus normal state; etc.). It is to be understood that also when referring to proteins (e.g. differentially expressed proteins), such may fall within the definition of “gene” signature, and may on certain occasions be referred to as “protein signature”.

### Kits

Kits are also provided herein. The kit can include primers, adaptors, terminal deoxynucleotidyl transferases (TdT), amplification reagents and other components suitable for use in the methods, e.g. ligases, polynucleotide kinases, fixative agents and the like.

### EXAMPLES

### Example 1: Co-Profiling of Chromatin Occupancy and RNAs in Single Cells

Methods for simultaneous profiling of chromatin occupancy and RNA in the same single cell are not available currently. Here, a technique, termed scPCOR-seq (single-cell Profiling of Chromatin Occupancy and RNAs Sequencing), is reported for simultaneously profiling genome-wide chromatin protein binding or histone modification marks and RNA expression in the same cell.

### Materials and Methods

Reagents. Histone H3 trimethyl Lys4 antibody was purchased from Millipore (catalog no. 07473), RNAPII antibody was purchased from Abcam (catalog no. ab817). Methanol-free formaldehyde solution was purchased from Thermo Fisher Scientific (catalog no. 28906). Terminal Transferase was purchased from New England BioLabs (catalog no. M0315L). The human embryonic stem cell line H1 (WA01- lot WB35186 p30) was provided by WiCell Research Institute. PA-MNase was purified after transformation of PET15b-PA-MNase plasmid (Addgene#124883) into BL21 Gold (DE3) following standard protocol.

Cell culture and fixation. HEK293T cells and GM12878 were maintained in DMEM (Invitrogen, catalog no. 10566-016) supplemented with 10% FBS (Sigma-Aldrich, catalog no. F4135-500ML) following standard procedure. The HI human embryonic stem cell line was maintained in feeder-free mTeSR™1 medium (Stem Cell Technologies, catalog no.85850) and passaged with ReLeSR™ (Stem Cell Technologies, catalog no.05872) following the manufacturer's instruction. Cells were harvested, washed with 1× PBS twice, and resuspended in DMEM containing 10% FBS and 1% formaldehyde. After 5 min incubation in room temperature, the reaction was stopped by adding 1.25 M glycine, followed by two rounds of washes with PBS. The cells were aliquoted into 1×10⁶ cells per tube, frozen on dry ice, and stored at −80° C.

Antibody-guided MNase digestion and end repair. The fixed cells were thawed on ice. To prepare PA-MNase and antibody complex, 1 μl antibody and 3 μl PA-MNase were pre-incubated on ice in 4 μl antibody binding buffer (10 mM Tris-Cl (pH 7.5), 1 mM EDTA, 150 mM sodium chloride, 0.1% Triton X-100) for 30 min. Meanwhile, H1 fixed cells (1 million) and HEK 293T fixed cells (1 million) were resuspended in 100 μl antibody binding buffer. Then, cell suspension was added to the PA-MNase and antibody complex, incubated on ice for 1 hour. Cells were washed three times with high salt buffer (10 mM Tris-Cl (pH 7.5), 1 mM EDTA, 400 mM sodium chloride and 1% (v/v) Triton X-100), followed by washing once with rinsing buffer (10 mM Tris pH7.5, 10 mM sodium chloride and 0.1% (v/v) Triton X-100). Then the cells were resuspended in 40 μl reaction solution buffer (10 mM Tris-Cl (pH 7.4), 10 mM sodium chloride, 0.1% (v/v) Triton X-100, 2 mM CaCl2), incubated at 37° C. for 3 min in water bath. The reaction was stopped by adding 4.4 μl 100 mM EGTA. After washing twice with rinsing buffer, the cells were end-repaired by T4 Polynucleotide Kinase (PNK) in 150 μl reaction buffer (1× PNK buffer, 1 mM ATP, 150 unites PNK) at 37° C. for 30 min, followed by washing twice with rinsing buffer to stop the reaction.

In-situ reverse transcription. The cells were resuspended in 25 μl reverse transcription buffer (5 μl 10× Maxima H Minus reverse transcription buffer, 1.25 μl 10% NP40, 16.75 μl H₂O, 1 μl 100 um not-so-random primers mixture ((Armour, C. D. et al. Digital transcriptome profiling using selective hexamer priming for cDNA synthesis. Nat Methods 6, 647-649, doi: 10.1038/nmeth.1360 (2009)), 1 μl 10 ng/μl Oligo dT22 primer (NNNNNNGAGCGTTTTTTTTTTTTTTTTTTTTTTVN)). After incubating at 65° C. for 1 min, the reaction was immediately put on ice, while the enzyme mix is prepared (8.75 μl H2O, 5 μl 10×Maxima H Minus reverse transcription buffer, 8 μl 10 mM dNTPs, 2 μl Maxima H Minus reverse transcriptase, 0.625 μl SUPERase·In™ RNase Inhibitor, 0.625 μl RNaseOUT™ Recombinant Ribonuclease Inhibitor) and added into the reaction. The reverse transcription was performed as described (Zhu, C. et al. An ultra high-throughput method for single-cell joint analysis of open chromatin and transcriptome. Nat Struct Mol Biol 26, 1063-1070, doi: 10.1038/s41594-019-0323-x (2019). (50° C.×10 min; 3 cycles for the following: 8° C.×12 s, 15° C.×45 s, 20° C.×45 s, 30° C.×30 s, 42° C.×2 min, 50° C.×5 min; 50° C.×10 min and hold at 4° C.

Exonuclease I (Exo I) digestion. The cells were washed twice with rinsing buffer, resuspended in 50 μl reaction buffer (5 μl 10×Exo I buffer, 1 μl Exo I, 44 μl H₂O) and incubated at 37° C. for 20 min. This is to remove the excess primers left after reverse transcription. After digestion, the cells were washed twice with rinsing buffer to stop the reaction.

Library construction. 96 barcode-P7 adaptors (10 μM) stored in a 96 well plate were thawed at 4° C., then 1 μl of each was added to the corresponding well in a new 96 well plate with multichannel pipette. Downstream library construction was performed as described previously for iscChIC-seq (Ku, Pan and Zhao et al., manuscript in revision). Briefly, the cells were suspended with nuclei suspension buffer and mixed with enzyme dilution buffer, followed by aliquoted into 10 μl in 96 wells, mixing with the added barcode-P7 adaptors. The plate was sealed completely and incubated at 37° C. for 60 min. After incubation, the cells were pooled together in a solution trough containing 500 μl stop buffer, resuspended with 800 μl 1× PBS and send to flow cytometry core. 30 cells were sorted in each well of a new 96 well plate which contain 13 μl buffer mixture per well (3 μl reverse-crosslink buffer, 10 μl PBS containing 0.1% NP40). The plate was sealed completely and incubated at 65° C. for 6 hours and 80° C. for 10 min.

After reverse crosslinking, indexed PCR1 was performed by adding 13 μl 2× PHUSION® High-Fidelity PCR Master Mix with HF Buffer (New England BioLabs) and 1 μl 2 μM index primer with the following condition: 98° C. 3 min, 12 cycles of 65° C. 30 s, 72° C. 30 s, followed by 72° C. 5 min. Then the libraries were pooled together, digested with Exo I and purified by MINELUTE® Reaction Cleanup Kit (Qiagen). Downstream A-tailing and P5 adaptor ligation were performed as described previously. PCR2 amplification with i5 index primer and P7-cs2 primer was set in the following condition: 98° C. 3 min, 57° C. 3 min, 72° C. 1 min, 7 cycles of 98° C. 10 s, 65° C. 15 s, 72° C. 30 s, followed by 72° C. 5 min. The PCR products were run on the 2% E-Gel® EX Agarose Gel (Invitrogen). The fragments between 250-600 base pair (bp) were isolated and purified by the MinElute Gel Extraction Kit (Qiagen). The concentration of the library was measured by Qubit dsDNA HS kit (Thermo Fisher Scientific). The paired-end sequencing was performed on Illumina Hiseq 2500 and Novaseq.

### Data Analysis

Pre-processing of scPCOR-seq and Reads mapping. Pairs of reads were considered to be valid if read 2 contained the exact linker sequences “AGAACCATGTCGTCAGTGT”. The valid pairs of read are further separated into either RNA part or chromatin occupancy part. If the linker sequences “GAGCG” for not-so-random primers or the linker sequences “CCTGCAGG” for oligodT were found in the location within 7-11^{th} and 7-14^{th} base of read 1, the pair of reads belonged to RNA. The remaining valid pairs belonged to chromatin occupancy. Using the information of the cell barcodes located at 5′ of read 2, both pairs of reads belonging to RNA and chromatin occupancy were separated into 96 sets of FASTQ files, respectively. Reads were mapped to the human reference genome hg19 using Bowtie2 Duplicates using different trimming parameters. Finally, the mapping results were combined, and Duplicated reads were removed based on mapping position and UMI for the reads belonging to chromatin occupancy.

Filtering for single cells and genes. For both scRNA-scRNAPII and scRNA-scH3K4me3 measurements. Genes and Peak regions were excluded if less than 6 cells or more than 300 cells have reads in these regions. If the cell-to-cell variation quantified by coefficient for the genes or peak regions are less than two, they were excluded, respectively. Single cells that have both at least 1000 RNA reads, and 1000 DNA reads were first considered. Also, if single cells have reads in less than 50 peak regions or 50 gene regions, they were excluded. Finally, the outlier cells, genes, peak regions were excluded, in which an outlier is a value that is more than three scaled median absolute deviations (Kaya-Okur, H. S. et al. CUT & Tag for efficient epigenomic profiling of small samples and single cells. (2019) Nat Commun 10, 1930, doi: 10.1038/s41467-019-09982-5) away from the median.

Cell Clustering. For either scRNA-scRNAPII or scRNA-scH3K4me3 measurements, the read count matrix for RNA was denoted as R′ while the read count matrix for DNA was denoted as D′. The columns of R′ correspond to cells and its rows correspond to the genes. Similarly, the columns of D′ correspond to cells and its rows correspond to the peak regions. Both of the read count matrices were normalized by the library sizes and were transformed by based two logarithm transformations. The final matrices are denoted as R and D for R′ and D′, respectively. For both RNA and DNA parts, the similarity between any two cells were computed using Pearson Correlation, resulting in two correlation matrices denoted as C^{R} and C^{D}, respectively. The Laplacian transformation was applied to the correlation matrices. The Laplacian matrix L is defined by L=1−T^{-1/2}AT^{-1/2}, where I is the identity matrix. A is a similarity matrix where A=e^{-(2-C)/max(2-C)}, C=CR or CD. Note that T is the Tis the degree matrix of A, a diagonal matrix that contains the row-sums of A on the diagonal (Dᵢᵢ=ΣᵢAᵢⱼ). The eigenvectors of the Laplacian matrix were computed and formed a matrix V where each column represents an eigenvector. The columns of V from left to right are sorted in ascending order based on their corresponding eigenvalues. For either RNA or DNA, a binary matrix E was considered in which its rows and columns correspond to single cells. The K-mean method was applied to the matrix W^{t} to cluster the single cells with k=2, where W^{t} is a submatrix V containing the first t columns. If cells i and j belong to the same cluster, Eᵢⱼ=Eⱼᵢ=1; otherwise 0. We consider t is between 2 to 15 and two consensus matrices E^{R} and E^{D}, correspond to RNA and DNA respectively, were calculated by averaging all binary matrices from each individual clustering. Finally, K-mean clustering was applied to the sum of E^{R} and E^{D} with k=2. Two set of cells determined by the clusters were obtained and denoted as K_{S}¹ and K_{S}².

PCA: For both RNA and DNA parts, principal component analysis (PCA) was applied to the two matrices to obtain the first 100 components. UMAP was further applied to the obtained principal component matrix. Cells were clustered for the scPCOR-seq cell line data. First, two cell-to-cell correlation matrices corresponding to RNA and DNA parts were computed using the obtained principal components. The z-score transformation was applied to these matrices (Faith, J. J., et al., Large-scale mapping and validation of Escherichia coli transcriptional regulation from a compendium of expression profiles. Plos Biology, 2007. 5(1): p. 54-66). The edges between two genes/regions with z-score values smaller than 3.2 were filtered out, resulting in two networks for RNA and DNA. The multiplex network clustering method MolTi (Didier, G., et al. Peerj, 2015. 3) was applied to both RNA and DNA networks.

Purity of clusters. The dimension reduction method t-SNE was applied to the two matrices R and D, with parameters ‘NumPCAComponents’ equal to 5 and 3, respectively. Also, the Perplexity for both reductions was equal to 100. For both RNA and DNA, two t-SNE component vectors were obtained as output. The K-mean clustering method was again separately applied to E^{R} and E^{D} with k=2. Two set of cells determined by each clustering were obtained. For RNA, they were denoted as K_{R}¹ and K_{R}². For DNA they were denoted as K_{D}¹ and K_{D}². The purity of K_{S}¹, i=1,2 is equal to

$$= \frac{max ( \vert {K}_{R}^{1} ∩ {K}_{S}^{i} \vert , \vert {K}_{R}^{2} ∩ {K}_{S}^{i} \vert ) + max ( \vert {K}_{D}^{1} {∩ K}_{S}^{i} \vert , \vert {K}_{D}^{2} ∩ {K}_{S}^{l} \vert )}{\vert 2 {K}_{S}^{i} \vert} .$$

CRE-gene correlation. Human Cis-regulatory elements were downloaded from ENCODE. The CRE regions that have reads in any cells in either H1 or 293 T cells were excluded. For each cell, the count of RNAPII binding in the CRE regions (+500) was computed and normalized by the library sizes. The Pearson correlation between the RNAPII density in each CRE region and the gene expression of each gene was calculated for both H1 and 293 T cells. Thus, for both H1 and 293 T cells, two correlation matrices with dimensions of number of CRE regions and number of genes were obtained. The negative elements were set to be equal to zero. A value is obtained for each CRE region by summing over all genes for the matrix subtracting between the two correlation matrices. Thus, CRE regions specific to H1 cells and 293T cells were obtained based on the values calculated.

Comparison between TrAC-looping data and CRE-gene interactions. First, the functional CRE-gene candidates were identified by requiring that both elements are on the same chromosome and the distance between CRE region and gene region is less than 100 kbp. A CRE-gene pair was H1 specific if its correlation between the RNAPII density and mRNA level is higher in H1 cells compared to 293T cells, and vice versa. Number of PETs from TrAC-looping data that connected the CRE region and gene region from each cell type specific CRE-gene interaction were counted. Note that a window size of 5 kb were used for the CRE regions and gene regions when comparing with the TrAC-looping data. The number of PETs were normalized by the total number of PETS in the library.

### Results

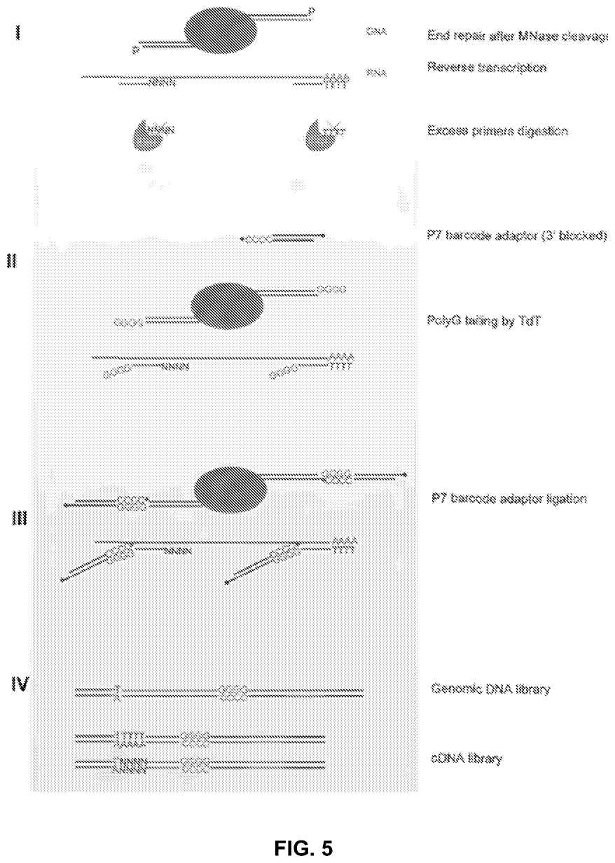

An indexing single-cell ChIC-seq (iscChIC-seq) protocol was developed to profile histone modifications, in which Terminal Transferase (TdT) was used to mediate dG tailing on MNase digestion sites, while oligo-dC protruding barcode adaptors were ligated to these sites by T4 Ligase. In order to capture both histone modification or protein occupancy on chromatin and RNA in the same cell, a strategy to detect RNA profiles simultaneously (FIG. 5). Briefly, Protein A-MNase (PA-MNase) was guided by specific antibodies to the targeted sites in formaldehyde-fixed cells. Following Ca²⁺-activated MNase digestion of chromatin, in situ reverse-transcription was performed by Maxima H Minus reverse transcriptase along with oligo dT primer and a mixture of 749 not-so-random primers that do not recognize rRNAs. Then both the MNase-digested sites and cDNA were tailed simultaneously by TdT and ligated with barcode adaptors in 96-well plate. The cells were pooled and sorted into a new 96-well plate with 30 cells per well by flow cytometry sorting, followed by two consecutive rounds of indexed PCR and final library sequencing. Single cells were resolved by identifying the unique combinations of barcodes and indexes as previously reported (Buenrostro, J. D. et al. (2015) Single-cell chromatin accessibility reveals principles of regulatory variation. Nature 523, 486-490, doi: 10.1038/nature14590; Cusanovich, D. A. et al. (2015) Multiplex single cell profiling of chromatin accessibility by combinatorial cellular indexing. Science 348, 910-914, doi: 10.1126/science.aab1601).

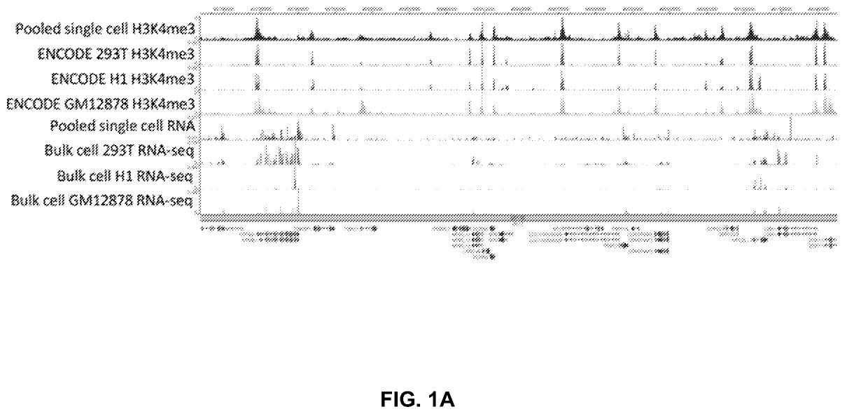

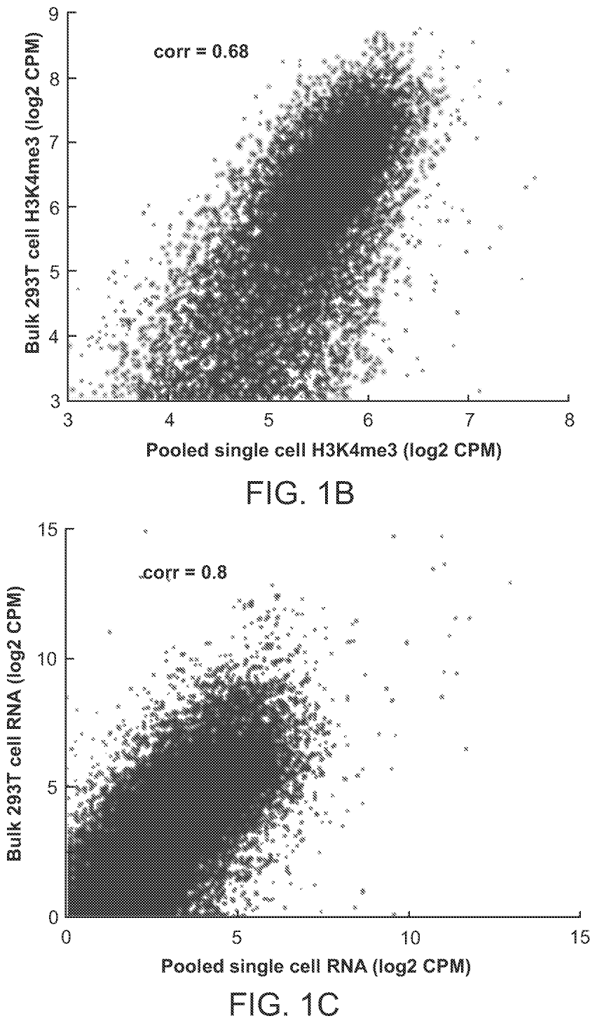

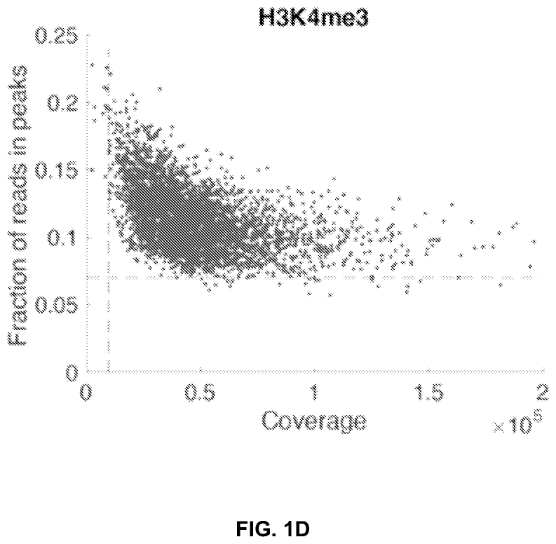

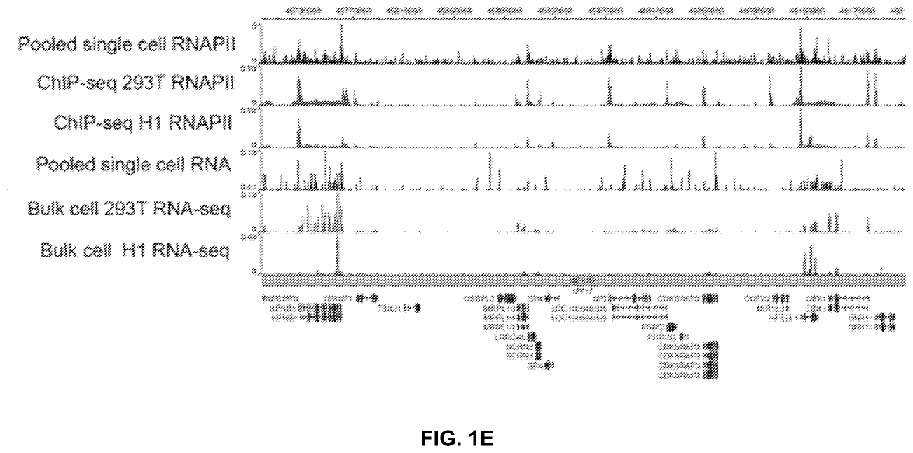

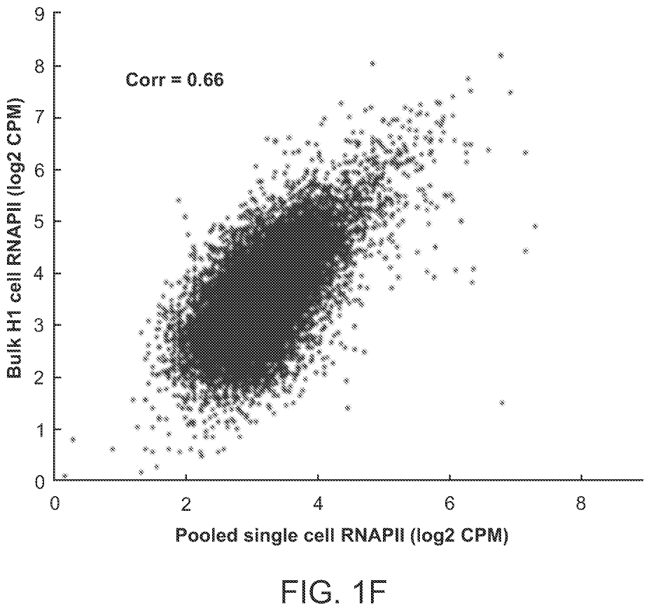

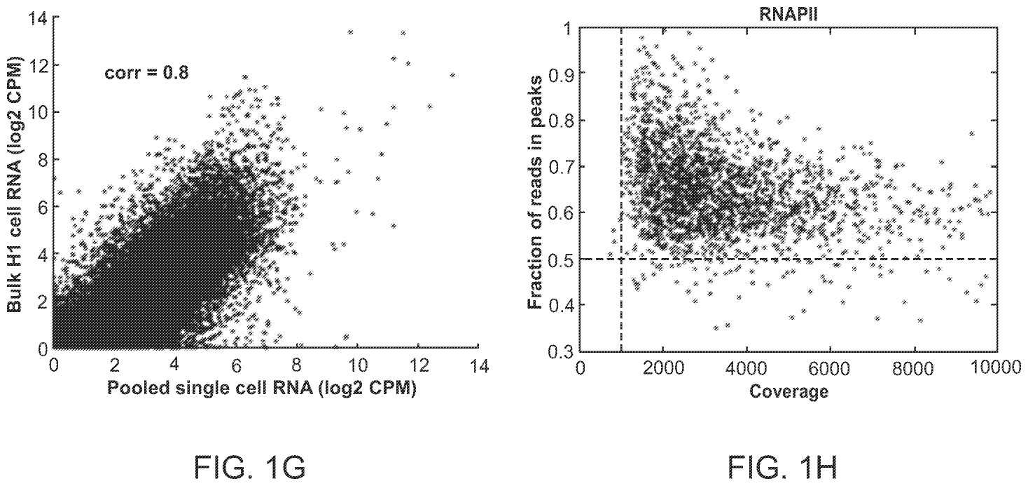

H3K4me3 and RNAs were profiled by applying scPCOR-seq to human 293T cells and mouse NIH 3T3 cells to estimate the detection of doublets. After identifying the barcodes that refer to cells in either RNA or H3K4me3 data, a collision rate of 0.08 was observed in the RNA data and a collision rate of 0.118 in the H3K4me3 data (FIG. 1J). The different number of reads in RNA and H3K4me3 may bring the discrepancy of collision rate between H3K4me3 and RNA data. However, collision rates obtained in both data suggest that the doublets rate in scPCOR-seq is comparable to previously published single-cell assays.

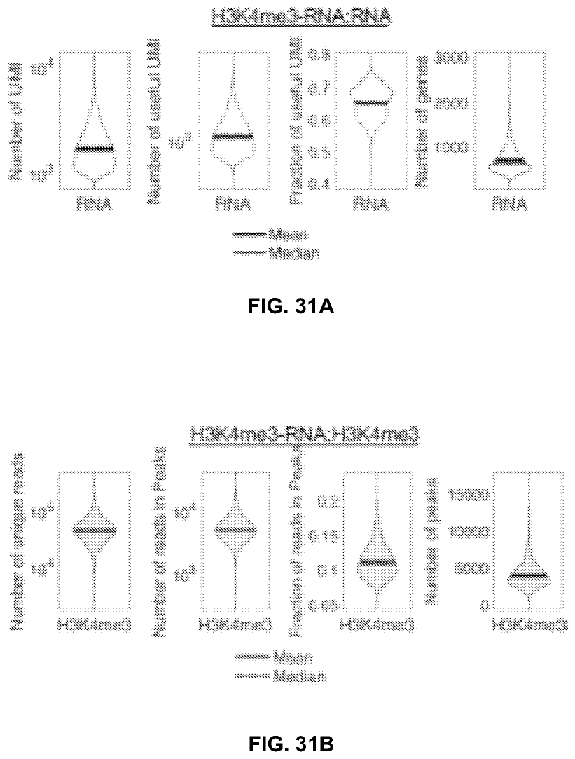

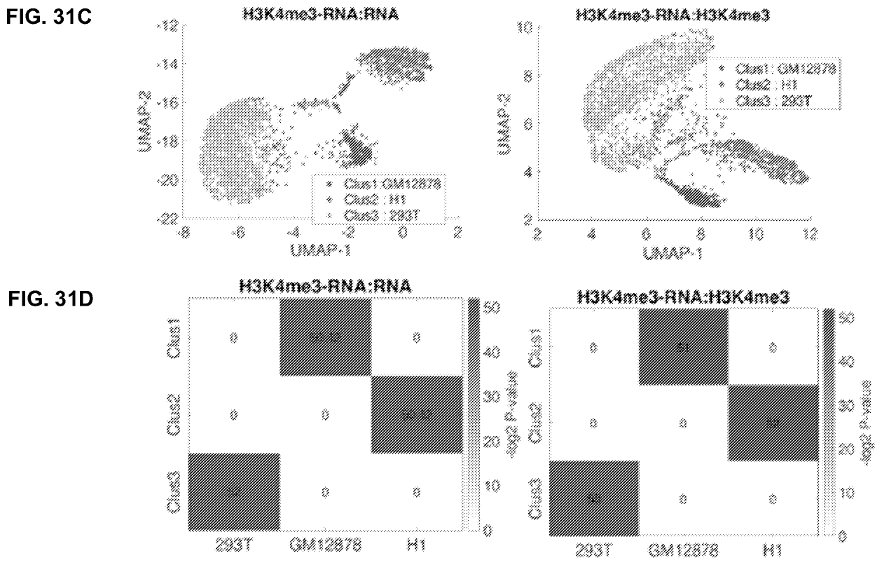

Next, H3K4me3 and RNAs were first profiled by applying scPCOR-seq to a mixture of human H1 ESCs, 293T cells, and GM12878 cells. After sequencing the libraries, the RNAs were distinguished from chromatin targets by a unique barcode embedded in the primers used for reverse transcription. 3,713 single cells were identified from the sequencing data (about 2,000 mRNA reads per cell and 45,000 H3K4me3 unique reads per cell). The H3K4me3 and RNA signals from the pooled single cells were compared with ENCODE H3K4me3 ChIP-seq data (FIG. 1A, top four tracks) and ENCODE RNA-seq data from H1 ESC and 293T cells (FIG. 1A, bottom four tracks), respectively. The quality of the single cell RNA-seq data was quantified by different metrics (FIG. 31A). A median of 1,300 (0.65 in terms of fraction) useful UMI (i.e, UMI located within gene regions) were detected per single cell. A median of 700 genes were detected per cell. Similarly, four metrics were used to quantify the quality of H3K4me3 signals. A median of 5,400 unique reads (0.12 in terms of fraction) per single cell were detected within the peaks identified using ENCODE data. A median of 3,000 peaks were detected per cell (FIG. 31B). Globally, the peaks from the pooled single cell H3K4me3 data showed a positive correlation of 0.71 with that from the ENCODE bulk 293T cell H3K4me3 ChIP-seq data (FIG. 1B); the RNA levels from the pooled single cells also demonstrated a high positive correlation (0.8) with that from bulk 293T cell RNA-seq data (FIG. 1C). More than about 7% of sequence reads fell into the H3K4me3 peaks in more than 90% of identified single cells (FIG. 1D). These results indicate that scPCOR-seq is able to simultaneously detect faithfully histone modification and RNA levels at a single-cell resolution.

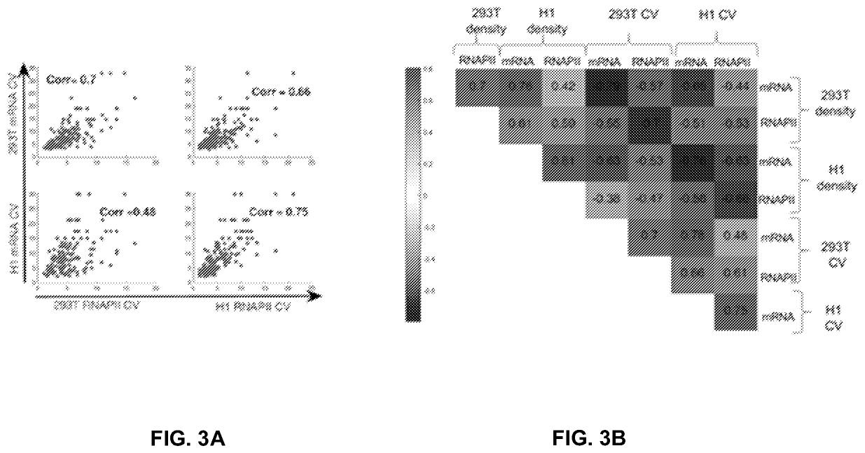

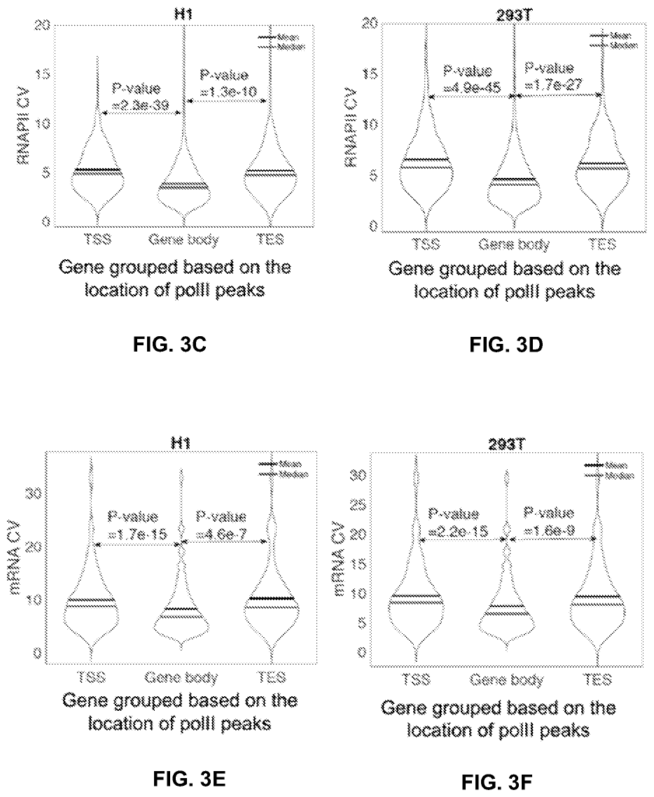

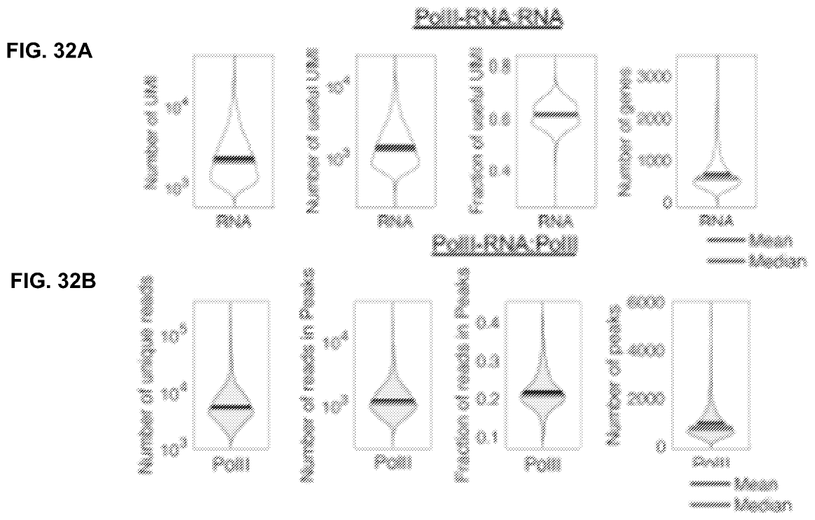

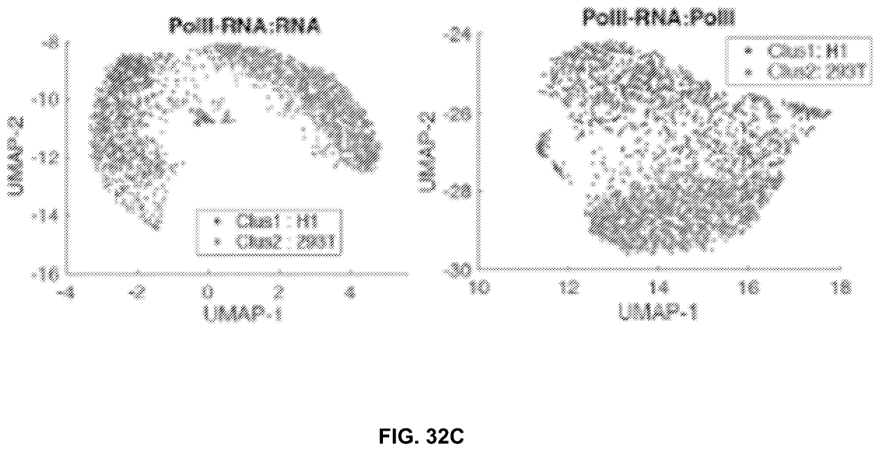

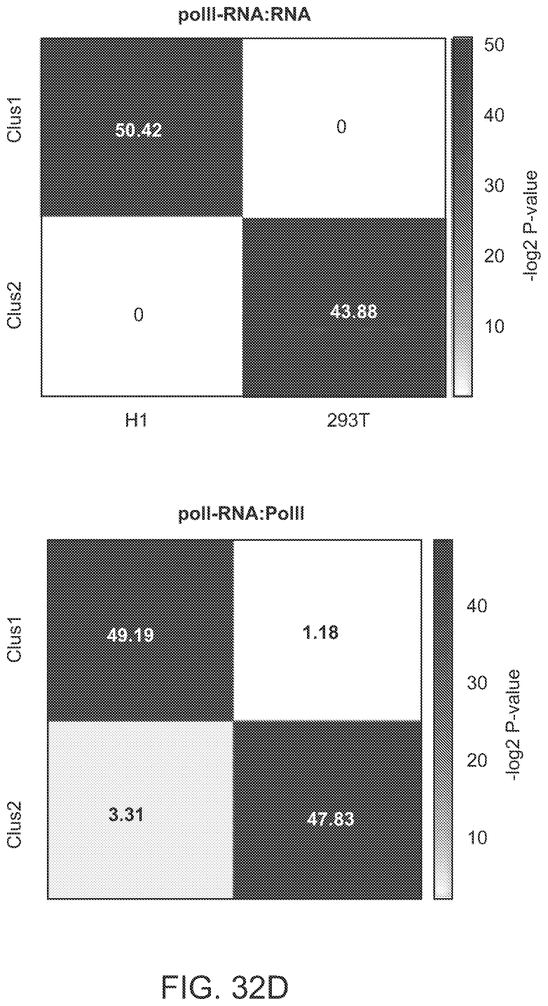

To test whether scPCOR-seq is able to detect chromatin binding proteins and RNAs in the same single-cell, it was applied to profile both RNA Polymerase II (RNAPII) binding and RNAs in a mixture of H1 ESCs and 293T cells. 2,347 single cells were identified from the sequencing data (about 3,000 mRNA reads per cell and 7,000 RNAPII unique reads per cell). The RNAPII binding and RNA signals from the pooled single cells were compared with ENCODE bulk cell RNAPII ChIP-seq data (FIG. 1E, top three tracks) and ENCODE RNA-seq data from H1 ESC and 293T cells (FIG. 1E, bottom three panels), respectively. A median of 1,900 (0.6 in terms of fraction) useful RNA UMI (i.e, UMI located within gene regions) were detected per single cell. A median of 700 genes were detected per cell (FIG. 32A). Also, four metrics were used to quantify the quality of RNAPII signals. A median of 1,400 unique reads (0.2 in terms of fraction) were located within the peaks identified using ENCODE data. A median of 900 peaks were detected (FIG. 32B). These results indicate that scPCOR-seq can simultaneously detect faithfully RNAPII binding and RNA levels at a single-cell resolution. A similar strategy was used to cluster cells based on the RNA-RNAPII co-profiling data (FIG. 32C). Both the single-cell RNA and RNAPII occupancy data correctly clustered H1 and 293T cells (FIG. 32D). Since RNAPII is directly responsible for producing RNAs and RNAPII binding from pooled single-cells in H1 and 293T cells indicates a positive correlation between RNAPII binding and RNA levels, it was next examined whether cell-to-cell variation in gene expression is correlated with that in RNAPII binding. The data indicate that cell-to-cell variation in gene expression is positively correlated with that in RNAPII binding in both H1 cells and 293T cells (FIG. 3A). Importantly, this correlation is cell type specific meaning that the correlation is higher if both gene expression and RNAPII data are from the same cell type. Globally, the analysis indicated that peaks from the pooled single cell RNAPII binding data showed a positive correlation of 0.66 with that from the ENCODE bulk H1 ES cell ChIP-seq data (FIG. 1F); the RNA levels from the pooled single cells also demonstrated a high positive correlation (0.8) with that from bulk HI cell RNA-seq data (FIG. 1G). More than 50% of sequence reads fell into the RNAPII peaks in more than 90% of identified single cells (FIG. 1H). These results indicate that scPCOR-seq is able to simultaneously detect faithfully RNAPII binding and RNA levels at a single-cell resolution.

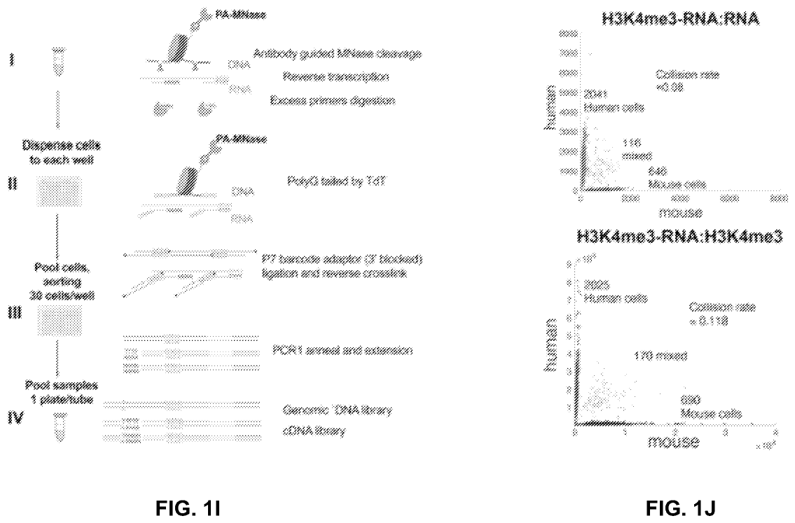

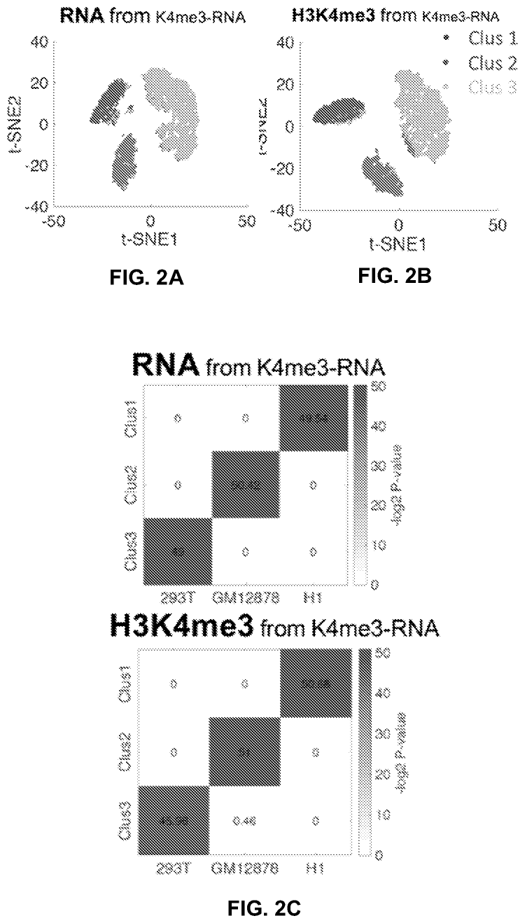

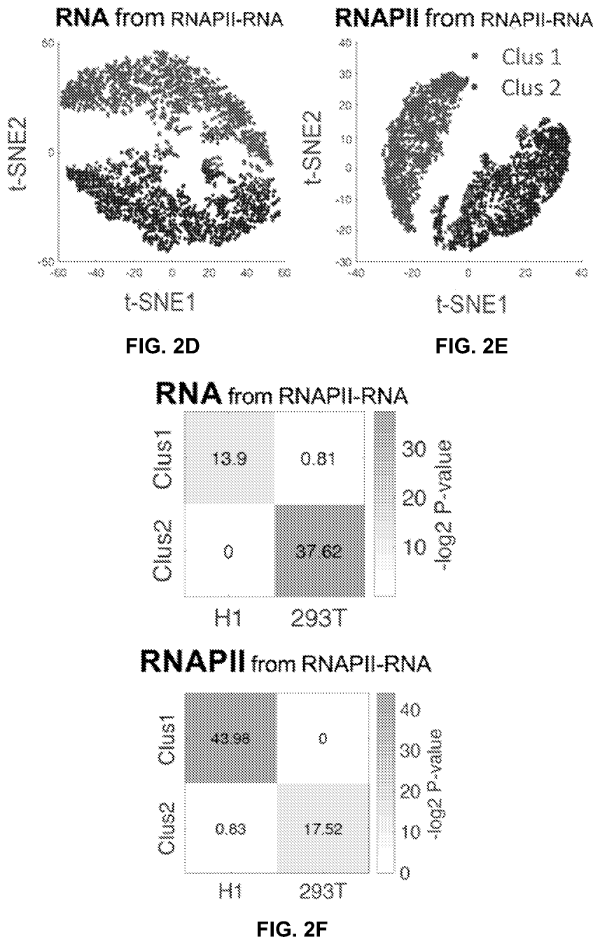

Next, to further validate the scPCOR-seq data, it was tested whether the single-cell RNA data or chromatin occupancy data from the assays can separate cells to different clusters. First, the dimension reduction t-SNE method was directly applied to the scPCOR-seq RNA and H3K4me3 data separately. The K-mean clustering method was applied to the reduced dimensions for clustering scRNA and scH3K4me3, separately. On the other hand, a consensus clustering approach was applied to both scRNA and scH3K4me3 data, from the RNA-H3K4me3 measurement. Single cells were separated into three clusters (Cluster 1 in blue, Cluster 2 in red, and Cluster 3 in orange) (FIGS. 2A and 2B). These results indicate that single cells can be clustered using either the RNA or H3K4me3 data independently from the scPCOR-seq measurement. However, it is not clear how consistent the results are between the RNA and H3K4me3-based clusters. To test the consistency, ground truth clustering results were first generated using the RNA and H3K4me3 data via a consensus approach. The consistency was tested using the quantity termed as the purity of clusters, which are defined as the fraction of cells overlap between the clusters identified using only RNA or H3K4me3 and the ground truth clustering results. The analysis revealed that the purity of clusters is all higher than 91%, providing evidence that both the RNA and H3K4me3 data of the scPCOR-seq assay are able to robustly separate different cell types. The clusters were annotated by comparing to the specifically expressed genes (FIG. 2C, upper panel) or specific H3K4me3 peaks (FIG. 2C, lower panel). The data indicate that Cluster 1, Cluster 2, and Cluster 3 are H1, GM12878, and 293T cells, respectively (FIG. 2C). A similar strategy was used to cluster cells based on the RNA-RNAPII co-profiling data (FIGS. 2D and 2E). Both the single-cell RNA and RNAPII occupancy data correctly clustered H1 and 293T cells (FIG. 2F).

The scPCOR-seq data was further validated by testing whether the single-cell RNA data or the H3K4me3 data from the assays can separate cells to different clusters. First, the PCA was directly applied to the scPCOR-seq RNA and H3K4me3 data separately. UMAP was applied to the reduced dimensions for scRNA and scH3K4me3, separately. Finally, the software MolTi (Didier, G., et al. Identifying communities from multiplex biological networks. Peerj, 2015. 3.) (multiplex-modularity with the adapted Louvain algorithm to cluster single cells using both RNA and

H3K4me3 data. Single cells were separated into three clusters (Cluster 1 in blue, Cluster 2 in red, and Cluster 3 in orange) from each dataset (FIG. 31C). The clusters were annotated by comparing to the specifically expressed genes (FIG. 31D, left panel) or specific H3K4me3 peaks based on the ENCODE data (FIG. 31D, right panel). The data indicate that Cluster 1, Cluster 2, and Cluster 3 are H1, GM12878, and 293T cells, respectively (FIG. 31D). These results indicate that both the RNA and H3K4me3 data from the scPCOR-seq assay can correctly separate different cell types from a mixture of cells.

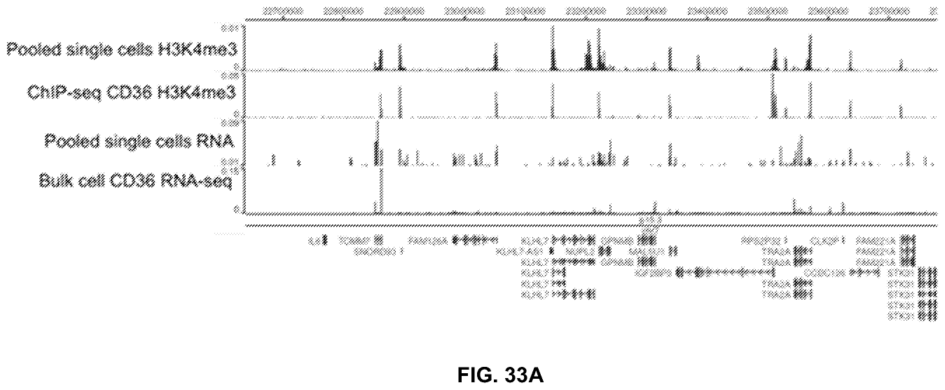

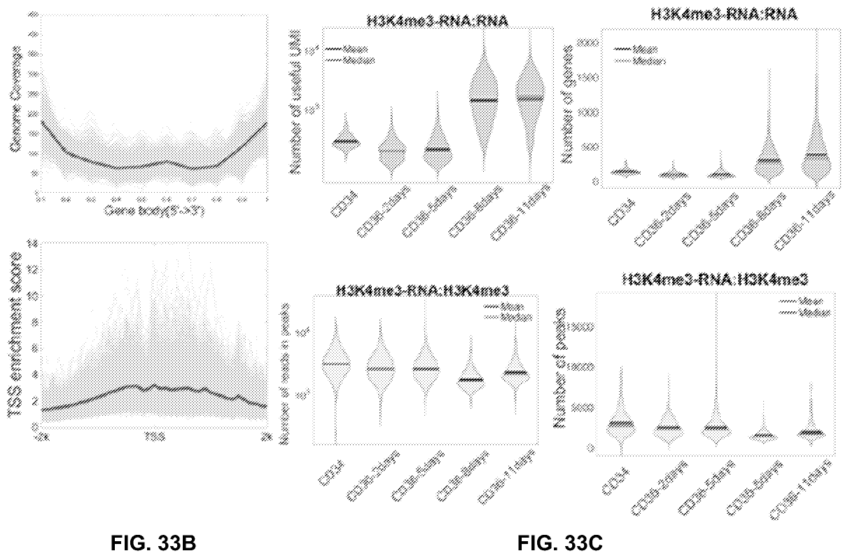

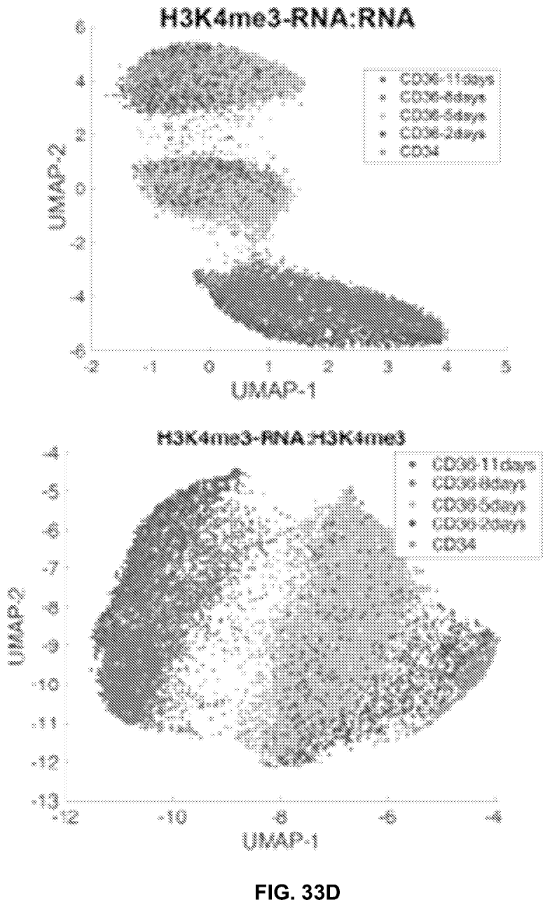

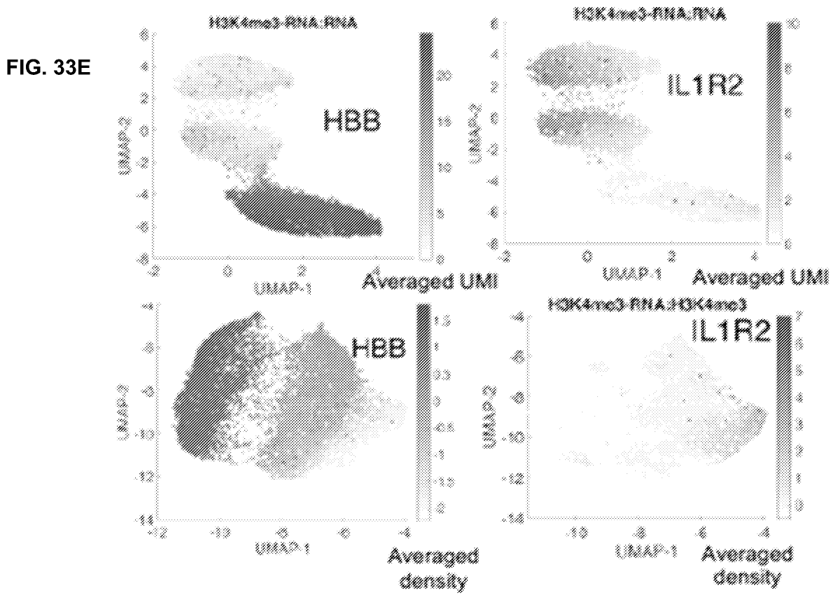

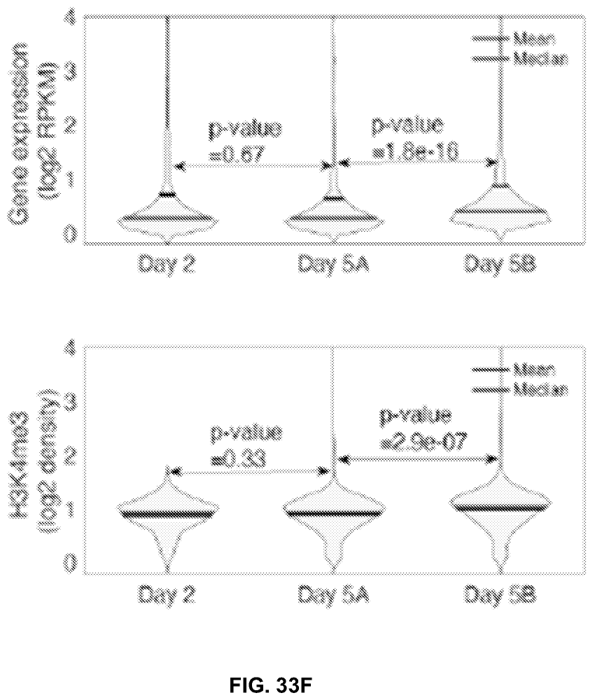

To test whether scPCOR-seq can be used to analyze more complex systems, it was applied to examining the in vitro differentiation of CD36+ erythrocyte precursor cells from human CD34+ hematopoietic stem/progenitor cells (Cui, K. R., et al., Cell Stem Cell, 2009. 4(1): p. 80-93). During the differentiation, the cell surface marker CD36 was significantly upregulated from day 5 and reaches peak expression by day 11, which is accompanied decreased expression of CD34. Libraries were constructed for both H3K4me3 and RNA for CD34+ cells and the cells differentiated for 2, 5, 8 and 11 days. The H3K4me3 and RNA signals from the pooled single cells (CD36+11 days differentiation) were compared with the published bulk cell H3K4me3 ChIP-seq data (FIG. 33A, the second tracks counted from the top) and with the published bulk cell RNA-seq data from CD36+ cells (FIG. 33A, bottom track). From the genome coverage profile of the RNA-seq data, the reads are more likely to be located at the TSS and TES regions (FIG. 33B, top panel). The enrichment plot of H3K4me3 data (FIG. 33B, bottom panel) around TSS showed the average fold-enrichment of 2.5. For the RNA-seq data, the median of the useful UMI increased from CD34+ cells (about 300 UMI) to CD36 cells at 11 days (about 3,000 UMI) (FIG. 33C, top left panel). The number of detected genes also increased from CD34+ cells (about 200 genes) to CD36+ cells at 11 days (about 500 genes) (FIG. 33C, top right panel). For the H3K4me3 data, the median of unique reads in peaks decreased from CD34+ cells (about 12,000 unique reads) to CD36+ cells at 11 days (about 7,000 unique reads) (FIG. 33C, bottom left panel). The number of detected peaks also decreased from CD34+ cells (about 3,000 peaks) to CD36+ cells at 11 days (about 1,200 peaks) (FIG. 33C, bottom right panel). The different numbers in the metrics among the cells at different differentiation stages are possibly due to the differences in cellular environments. Next, single cells were clustered and projected into the reduced space from UMAP (FIG. 33B). It was observed that the CD34+ cells and day 11 CD36+ cells were localized to two clusters that are most distant from each other in the plot with ether RNA or H3K4me3 data, which is consistent with the process of cell differentiation. The clusters of day 8 and day 11 CD36+ cells based on either RNA or H3K4me3 were very close to each other in the plot, indicating a high similarity between them. The day 2 CD36 cells exhibited high levels of heterogeneity in both the RNA and H3K4me3 plots, suggesting that the cells display heterogeneous levels of response to differentiation signals at the early stages of differentiation. Interestingly, the H3K4me3 data of day 5 CD36 cells displayed different patterns of clustering properties as compared to the RNA data. It was apparent that the day 5 CD36 cells based on the H3K4me3 data already exhibited a unique cluster that was localized between the clusters of CD34/CD36 (day 2) and CD36 (day 8 and 11) cells (FIG. 33D, lower panel). However, clustering of the day 5 CD36 cells based on the RNA data separated the cells into two distinct clusters: one was localized between the clusters of CD34/CD36 (day 2) and CD36 (day 8 and 11) cells while the other was not separated from the CD34/CD36 (day 2) cells (FIG. 33D, upper panel). These results provide evidence that the changes in H3K4me3 may occur ahead of the changes in transcription during the differentiation process, implying that H3K4me3 plays a critical role in cell differentiation process which later controls the transcription landscape. Different cell type specific genes were selected (HBB is more specific in CD34 cells while ILIR2 is more specific in CD36). Their expression level and H3K4me3 density were shown in the UMAP spaces in which the change is also consistent to their cell-type specific roles (FIG. 33E).

As shown in FIG. 33D, the cells at CD36 5 days were clustered into two groups using K-means method using the RNA data. The two clusters of cells were named as CD36 5days-A and CD36 5 days-B. The cells in CD36 5days-A are more like CD34 cells and CD36 2 days cells. Compared to Day 5A cells, 341 genes have higher expression in Day 5B cells while no genes has lower expression in Day 5B cells (FIG. 33F, upper panel). At the same time, the H3K4me3 density at these genes also showed increased H3K4me3 signals from Day 5A to Day 5B cells (FIG. 33F, lower panel).

Finally, the accessibility bias was examined in the H3K4me3 data by comparing the H3K4me3 with H3K4me3 ChIP-seq data and ATAC-seq data in CD36+ cells. The H3K4me3 data from scPCOR-seq data is highly consistent with H3K4me3 ChIP-seq data instead of the ATAC-seq data.

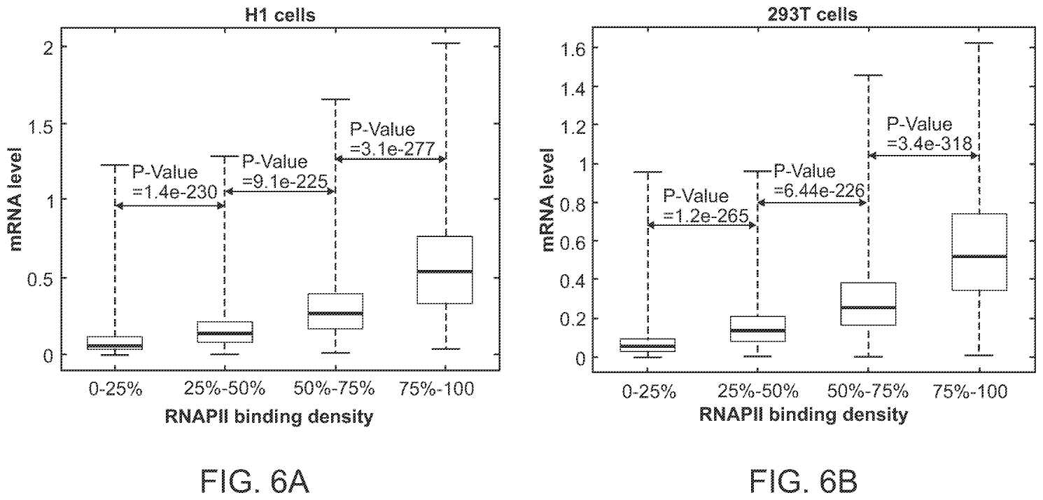

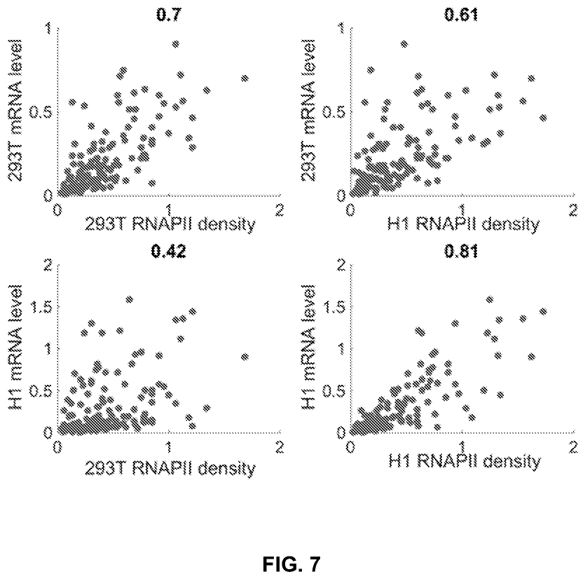

Since RNAPII is directly responsible for producing RNAs and RNAPII binding from pooled single-cells in H1 and 293T cells indicate a positive correlation between RNAPII binding and RNA levels (FIGS. 6A, 6B), it was next examined whether cell-to-cell variation in gene expression is correlated with that in RNAPII binding. The data indicated that cell-to-cell variation in gene expression is positively correlated with that in RNAPII binding in both H1 cells and 293T cells (FIG. 3A). Importantly, this correlation is cell type specific meaning that the correlation is higher if both gene expression and RNAPII data are from the same cell type. Besides cell-to-cell variation, the data also indicated that the mRNA level is also cell-type specifically correlated to the RNAPII density for both H1 and 293 T cells (FIG. 7). In addition, the data showed that cell-to-cell variation is negatively correlated with RNA and RNAPII density, which is consistent with previous findings (Ku, W. L. et al. (2019) Single-cell chromatin immunocleavage sequencing (scChIC-seq) to profile histone modification. Nat Methods 16, 323-325, doi: 10.1038/s41592-019-0361-7). This negative correlation is specific to both cell types and assays as shown by the high negative correlation in the diagonal of the blue blocks (FIG. 3B). The regulation of RNA production by RNAPII involves several steps including binding to gene promoters and transcription initiation, elongation with RNAPII traveling through the gene body, and transcription termination when RNAPII is associated at the 3′ end of genes. RNAPII can be captured at any of these moments in different single cells by scPCOR-seq. Thus it was examined whether the heterogeneity in RNAPII binding change during transcription and how it correlates with the cellular heterogeneity in RNA levels. For this purpose, genes were separated in three groups based on the location where RNAPII binding was detected: (1) in the promoter region (+/-2 kb surrounding TSS), (2) in the gene body region, and (3) in the 3′ ends of genes (+/−2 kb surrounding TTS). First analyzed was the cellular heterogeneity in RNAPII binding and it was found that the cell-to-cell variation in RNAPII binding is higher for the genes with RNAPII peak in the promoter region than the genes with RNAPII peak in gene body regions; the variation in RNAPII binding is also higher for the genes with RNAPII peak in 3′ gene ends than the genes with RNAPII peak in the gene body region (FIGS. 3C and 3D). These results provide evidence that RNAPII bound at different genomic regions may contribute differently to the expression variation across different cells. To test this idea, the cellular heterogeneity in gene expression in these three groups of genes was examined and it was found that the cell-to-cell variation in RNA levels is higher for the genes with RNAPII peak in the promoter region than the genes with RNAPII peak in gene body regions; and interestingly, the cell-to-cell variation in RNA levels is also higher for the genes with RNAPII peak in the 3′ gene ends than the genes with RNAPII peak in the gene body region (FIGS. 3E and 3F). These results indicate that the cellular heterogeneity in RNAPII binding is positively correlated with that in gene expression and RNAPII binding at the TTS regions also contributes to cellular heterogeneity in gene expression.

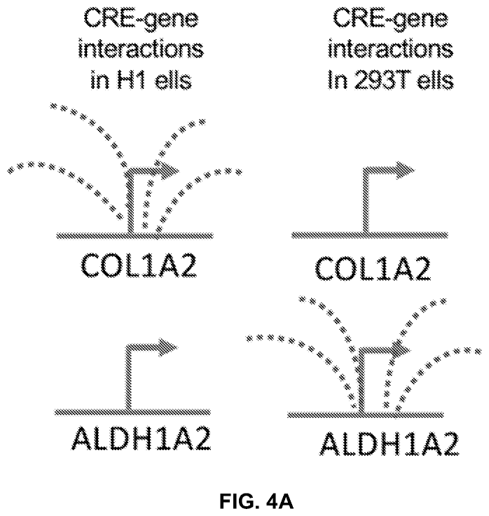

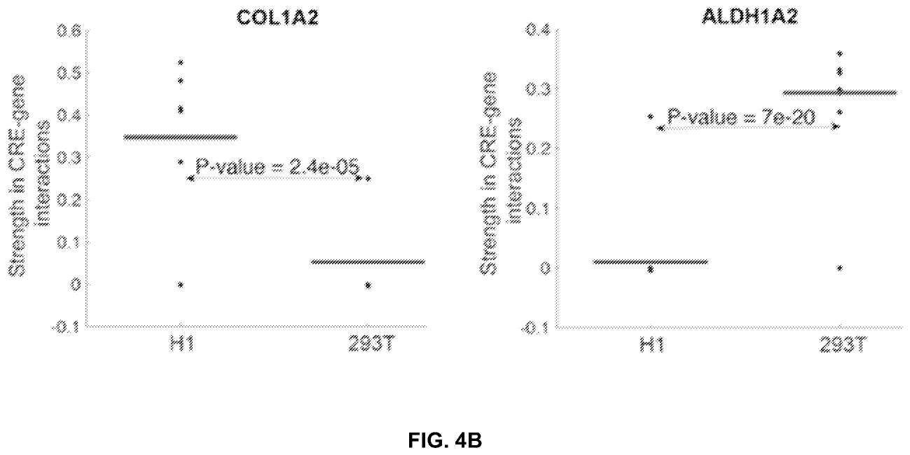

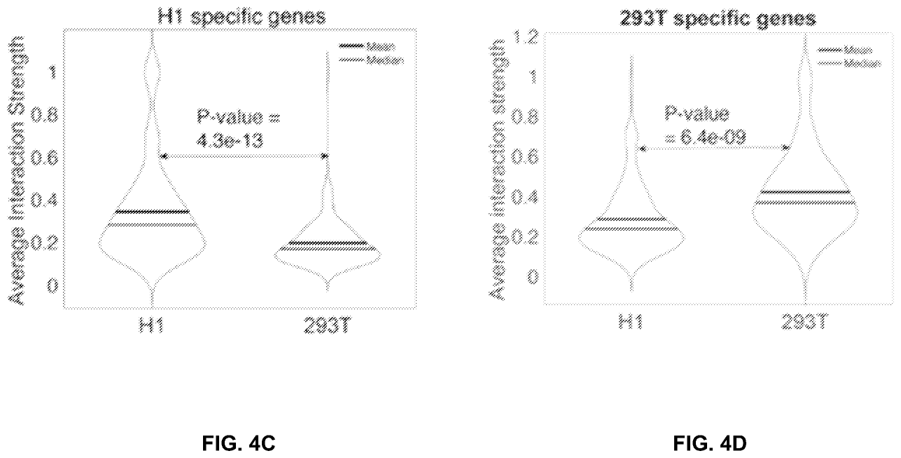

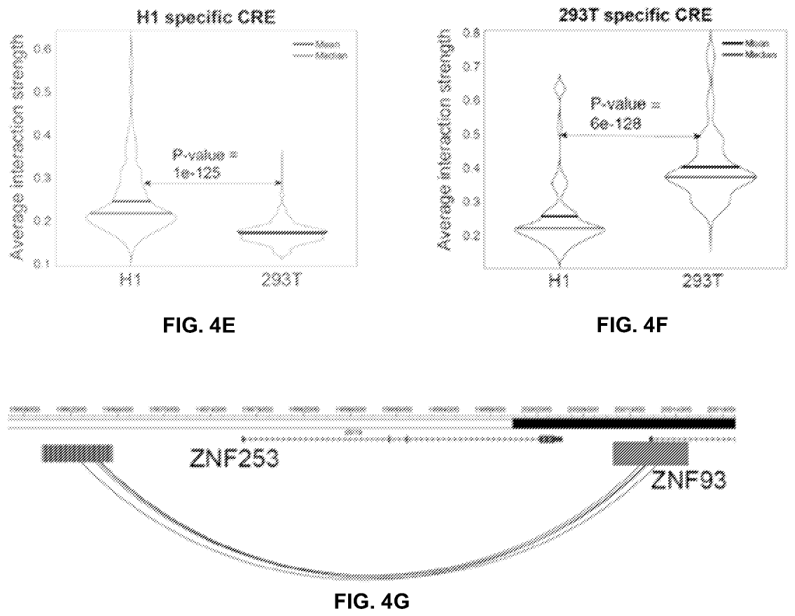

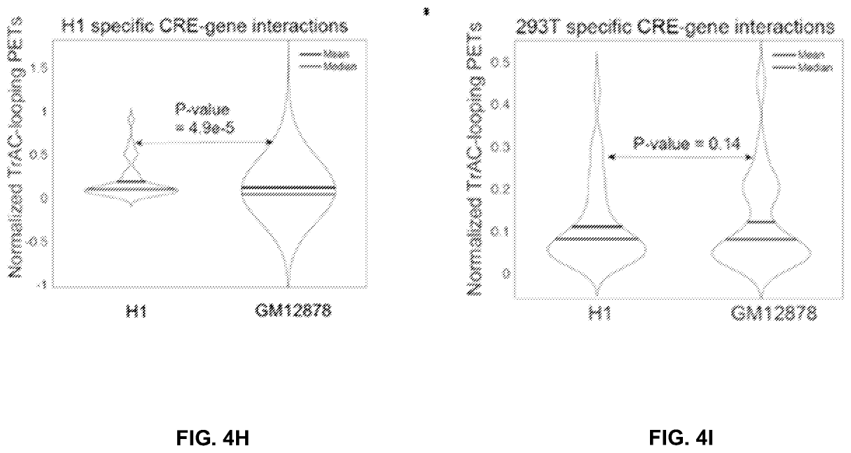

In addition to promoters and transcribed regions, RNAPII is associated with cis regulatory elements (CREs) such as enhancers of active genes (De Santa, F. et al. (2010) A large fraction of extragenic RNA pol II transcription sites overlap enhancers. PLOS Biol 8, e1000384, doi:10.1371/journal.pbio.1000384). Thus, co-binding to CREs and genes may provide evidence of a functional interaction relationship. To this end, the candidate CREs were downloaded from the ENCODE database (Roadmap Epigenomics, C. et al. (2015) Integrative analysis of 111 reference human epigenomes. Nature 518, 317-330, doi:10.1038/nature14248). By considering a window of 1000 bp for each element, the RNAPII density at the CREs and the correlation between the RNAPII density at CRE and gene expression level for both H1 and 293T cells was computed. A pair of CRE and gene is considered to be functionally interacting if the correlation between RNAPII density and gene expression level is higher than a cutoff. Therefore, H1 and 293T cells can have different interactions between CRE regions and genes (FIG. 4A). First, genes in the CRE-gene interaction pairs were examined. It was found that there are more CRE-gene interactions in H1 cells than those in 293T cells for genes such as COLIA2, which are specifically expressed in HI cells (FIG. 4B, left). Similarly, there are more CRE-gene interactions in 293T cells than those in H1 cells for genes such as ALDHIA2, which are specifically expressed in 293T cells (FIG. 4B, right). In general, genes were identified that are specially expressed in H1 and 239T cells and computed the average interaction strength, which is the average correlation values of interaction, for the genes. The data indicate that the average interaction strength is significantly stronger in H1 cells than in 293T cells for HI specific genes, and vice versa (FIGS. 4C and 4D). These results provided evidence that the CRE-gene interactions are also cell-type specific. Second, the CRE regions in the CRE-gene interaction pairs were examined. Based on the CRE-gene interactions in H1 cells and 293T cells, CREs that are specific to H1 and 293T cells, respectively were identified. The data indicate that the average interaction strength is significantly stronger for H1-specific CREs in H1 cells than in 293T cells, and vice versa (FIGS. 4E and 4F). These results indicate that co-profiling of RNA and RNAPII binding in single cells provides an approach for prediction of CREs associated with cell-to-cell variations in gene expression.

Enhancers regulate their target gene expression by direct physical interaction with target promoters. Thus, the functional interaction between the CRE-gene pairs discovered above could be facilitated by direct physical interaction. To further test this hypothesis, the physical chromatin interaction between the CRE-gene pairs was examined using TrAC-looping data, which specifically detects chromatin interactions among accessible chromatin regions (Lai, B. et al. (2018) Principles of nucleosome organization revealed by single-cell micrococcal nuclease sequencing. Nature 562, 281-285, doi: 10.1038/s41586-018-0567-3). Since most enhancer-promoter interactions occur within a range of 100 kb (van Arensbergen, J., van Steensel, B. & Bussemaker, H. J. (2014) In search of the determinants of enhancer-promoter interaction specificity. Trends Cell Biol 24, 695-702, doi: 10.1016/j.tcb.2014.07.004), this category of functional CRE-gene interactions was focused on by selecting the CRE-TSS pairs that have a distance shorter than 100 kb. Next, the H1-specific and 293T-specific CRE-gene interaction pairs were identified and their respective physical interaction strength was examined using the H1 cell TrAC-looping data. The results showed that the normalized TrAC-looping PETs from H1 cells were significantly higher at the H1-specific CRE-gene pairs than the 293T-specific pairs (FIGS. 4G and 4H). In comparison, the TrAC-looping data from an irrelevant cell line, GM12878, did not show different interaction intensity between the two groups of CRE-gene pairs (FIG. 4I). These results provide additional evidence of function for the CRE-gene interaction pairs identified from the co-profiling of RNA and RNAPII binding in single cells.

Elucidating cellular heterogeneity was shown to be important for understanding different biological processes, including cell differentiation and tumor progression etc. However, few studies addressed the question of origins and mechanisms of cellular heterogeneity in gene expression. A number of studies indicated variations in chromatin status may contribute to variations in gene expression, suggesting that both cis regulatory elements and trans acting chromatin binding factors play important roles in the cellular heterogeneity of gene expression. In this study, scPCOR-seq was developed, a method for simultaneously measuring RNA expression levels and chromatin occupancy of chromatin binding proteins or histone modifications in the same single cell and demonstrated its application to human H1 ESCs, GM12878, and 293T cells. Analysis of the data revealed that a differential correlation between the location of RNAPII binding and the cell-to-cell variation in gene expression and many CREs co-bound by RNAPII. Overall, it was concluded that scPCOR-seq will serve as a new powerful tool to study the relationship between different omics-layers and the mechanisms behind cellular heterogeneity.

### Example 2: Profiling Single Cell Histone Modifications Using Indexing Chromatin Immunocleavage Sequencing (iscChIC-seq)

In this study, an assay, termed herein “iscChlC-seq” was developed to profile histone modification marks in single cells. This technique employs the highly efficient TdT enzyme combined with T4 DNA ligase to add a unique barcode to the DNA ends generated by antibody-guided MNase cleavage in each cell. Using iscChIC-seq, the active histone modification mark H3K4me3 and repressive histone mark H3K27me3 were profiled in more than 10,000 single human white blood cells for each modification with detection of about 11,000 and 45,000 reads per cell, respectively, the largest cell number and read number compared to other current high-cell throughput methods. The data allowed successful clustering of different immune cells including T, B, NK, and monocytes from human WBCs. It was found that cell-to-cell variations in H3K4me3 and H3K27me3 in bivalent domains are positively correlated. The cell types annotated from H3K4me3 single cell data are specifically correlated with the cell types annotated from H3K27me3 single cell data. Overall, it was concluded that iscChlC-seq is a reliable method for studying histone modifications at the single cell level, which provide important information for the differentiation status of cells.

### Materials and Methods

### Reagents

Histone H3 trimethyl Lys4 antibody were purchased from Millipore (catalog no. 07-473), histone H3 trimethyl Lys27 antibody were purchased from Diagenode (catalog no. pAb-069-050). Methanol-free formaldehyde solution and DSG (disuccinimidyl glutarate) were purchased from Thermo Fisher Scientific (catalog no. 28906, 20593). Terminal Transferase was purchased from New England BioLabs (catalog no. M0315L). The human embryonic stem cell line H1 (WA01—lot WB35186 p30) was provided by WiCell Research Institute. PA-MNase was purified after transformation of PET15b-PA-MNase plasmid (Addgene#124883) into BL21 Gold (DE3) following standard protocol.

### Cell Culture and Fixation

HEK293T cells and GM12878 were maintained in DMEM (Invitrogen, catalog no. 10566-016) supplemented with 10% FBS (Sigma-Aldrich, catalog no. F4135-500ML) following standard procedure. The H1 human embryonic stem cell line was maintained in feeder-free mTeSR™1 medium (Stem Cell Technologies, catalog no.85850) and passaged with ReLeSR™ (Stem Cell Technologies, catalog no.05872) following the manufacturer's instruction. Cells were harvested, washed with 1× PBS twice, and resuspended in DMEM containing 10% FBS and 1% formaldehyde. After 5 min incubation in room temperature, the reaction was stopped by adding 1.25 M glycine, followed by two rounds of washes with PBS. The cells were aliquoted into 1×10⁶ cells per tube, frozen on dry ice, and stored at −80° C.

### PA-MNase Induction and Purification

PET15b-PA-MNase plasmid (Addgene#124883) was transformed into BL21 Gold (DE3) following standard protocol and grow in 40 ml LB medium (containing Ampicillin) overnight. Culture was diluted (1:50) into prewarmed LB medium (containing Ampicillin) and shake for 2 hours at 37° C. till OD₆₀₀ reached ˜0.6. Fresh IPTG was added to the culture to final 1 mM and shake for another 2.5 hours. For PA-MNase purification, cells pellet was collected, resuspended in 30 ml lysis buffer (50 mM NaH₂PO₄, 300 mM NaCl, 10 mM Imidazole, 1× EDTA-free protease inhibitor cocktails, 0.5 mM PMSF) supplemented with 30 mg Lysozyme (Thermo Fisher Scientific) and incubated on ice for 30 min. Cell lysate was sonicated for 10 cycles (10 sec on, 10 sec off) and centrifuged at 10,000g for 20 min. In the meantime, 2 ml 50% bead slurry were washed with lysis buffer. Then the supernatant was collected, mixed with beads slurry and rotated at 4° C. for 1 h. After spinning down, the beads were washed 4 times with 8 ml wash buffer (50 mM NaH₂PO₄, 300 mM NaCl, 20 mM Imidazole, 1× EDTA-free protease inhibitor cocktails, 0.5 mM PMSF), followed by three times elution with elution buffer(50 mM NaH₂PO₄, 300 mM NaCl, 250 mM Imidazole, 1× EDTA-free protease inhibitor cocktails, 0.5 mM PMSF). The purified fraction was mixed with glycerol, finally aliquoted into small tubes and stored in −80° C.

### WBC Preparation

Human blood samples were obtained from healthy donors from the NIH Blood Bank. The WBCs were isolated as described (Ku W. L. et al. 2019. Single-cell chromatin immunocleavage sequencing (scChIC-seq) to profile histone modification. Nat Methods 16: 323-325). Two-step fixation was modified from(Tian et al. 2012) and performed at room temperature. First, 50 M cells were suspended in 50 ml PBS/MgC12 containing 2 mM DSG and rotated for 45 min. After washing with PBS, the cells were resuspended in 45 ml culture medium DMEM containing 10% FBS. 3 ml 16% formaldehyde was added to 1% final concentration and rotated for 5 min, then the reaction was stopped by adding glycine, followed by two times washes with PBS. The cells were aliquoted into 2×10⁶ cells per tube, frozen on dry ice, and stored at −80° C. until use.

### MNase Digestion

To prepare ProteinA-MNase and antibody complex, 10 μl antibody and 25 μl PA-MNase were pre-incubated on ice in 40 μl antibody binding buffer (10 mM Tris-Cl (pH 7.5), 1 mM EDTA, 150 mM sodium chloride, 0.1% Triton X-100) for 30 min. Meanwhile, the fixed cells (0.25 million) were thawed on ice and resuspended in 200 μl antibody binding buffer. For H3K27me3 analysis, chromatin need to be firstly decondensed by suspending the fixed cells in 0.5 ml RIPA buffer (10 mM Tris-Cl (pH 7.5), 1 mM EDTA, 150 mM sodium chloride, 0.2% SDS, 0.1% sodium deoxycholate, 1% Triton X-100) and incubated at room temperature for 10 min followed by a one time wash in 0.5 ml antibody binding buffer. Then the cells were mixed with PA-MNase and antibody complex, incubated on ice for 60 min, followed by three washes with 500 μl high salt buffer (10 mM Tris-Cl (pH 7.5), 1 mM EDTA, 400 mM sodium chloride and 1% (v/v) Triton X-100). After washing in 200 μl rinsing buffer (10 mM Tris pH7.5, 10 mM sodium chloride and 0.1% (v/v) Triton X-100), the 336 cells were resuspended in 40 μl reaction solution buffer (10 mM Tris-Cl (pH 7.4), 10 mM sodium chloride, 0.1% (v/v) Triton X-100, 2 mM CaCl₂) to activate MNase digestion and incubated at 37° C. for 3 min in water bath. The reaction was stopped by adding 4.4 μl 100 mM EGTA. The cells were pelleted by centrifugation at 500 g for 5 min.

### TdT and T4 Ligation

The MNase cleavage sites were end-repaired by T4 Polynucleotide Kinase (PNK) for removal of 3′-phosphoryl groups and addition of 5′-phosphates to allow subsequent polyG tailing and ligation. After digestion, the cells were washed twice with 1 ml 1× T4 ligase buffer containing 0.1% NP40, then suspended in 300 μl mixed T4 PNK buffer (1× T4 PNK buffer, 1 mM ATP, 30 μl T4 PNK enzyme) and incubated at 37° C. for 30 min. Meanwhile, 96 barcode-P7 adaptors were thawed, 2.5 μl 10 μM barcode-P7 adaptors were added to a new 96 well PCR plate with multichannel pipette (1 barcode per well). After incubation, the cells were washed once with 1 ml rinsing buffer, suspended with 516 μl nuclei re-suspension buffer (1.27× T4 ligase buffer, 2.5 mM dGTP, 0.05% NP40), and mixed with 526 μl enzyme dilution buffer (1.25× T4 ligase buffer, 52.5 μl Terminal Transferase, 78 μl T4 ligase). Then 10 μl cell suspension was aliquoted, mixed with the 2.5 μl barcode-P7 adaptor in each well. Finally, the 12.5 μl reaction mixture (1× T4 ligase buffer, 1 mM dGTP, 0.02% NP40, 0.5 μl Terminal Transferase, 0.75 μl T4 ligase) in the 96 well PCR plate was sealed completely and incubated at 37° C. for 60 min.

### Pool and split

After barcoding the MNase cleavage sites, the reaction system in the 96 wells were pooled together in a solution trough containing 500 μl stop buffer (10 mM Tris-HCl (pH 8.0), 150 mM NaCl, 10 mM EDTA, 0.1%(v/v) Triton X-100), the cells were pelleted, resuspended in 800 μl PBS and send to flow cytometry core. 30 cells were sorted in each well of a new 96 well plate using a BD FACSAria III cell sorter (BD Biosciences) and collected in 10 μl PBS containing 0.1% NP40. Totally 5 plates were collected. After adding 3 μl reverse-crosslink buffer (50 mM Tris-HCl (pH 8.0), 25 ng/ml Proteinase K and 0.1% NP40) into each well by multichannel pipette, the plates were sealed completely, incubated in PCR machine for 65° C. overnight and 80° C. 10 min to inactivate the Proteinase K.

### Library Preparation and Sequencing

After reverse-crosslink, the DNA fragments with barcode adaptors were captured and labeled with second library indexes through 12 cycles of annealing and extension with 96 PCR1 index primers. The reaction was carried out by adding 15 μl 2× PHUSION® High-Fidelity PCR Master Mix with HF Buffer (New England BioLabs) and 2.5 μl 2 μM index primer (1 index per well) into the reverse-crosslinked solution in 96 wells. Then all the libraries were pooled together as described above, digested 370 with 96 μl Exonuclease I (Thermo Fisher Scientific) at 37° C. for 30 min to degrade the excess index primers. The DNAs were purified by MINELUTE® Reaction Cleanup Kit (Qiagen) and eluted with 64 μl EB buffer (Qiagen). The A tailing was performed in 1× NEBuffer 2 (New England BioLabs) by adding the Klenow fragment (3′→5′ exo-) (New England Biolabs) and 1 mM deoxyATP (New England Biolabs). After incubation at 37° C. for 30 min, the DNAs were purified and eluted by 23 μl EB buffer. Then the Illumine P5 adaptor was ligated to the A-tailing fragments using the T4 DNA ligase (New England BioLabs) by incubation at 16° C. overnight. The DNAs were purified again and eluted by 15 μl EB buffer. PCR2 amplification was performed by adding the PHUSION® High-Fidelity PCR Master Mix with HF Buffer, i5 index primer and P7-cs2 primer in the following condition: 98° C. 3 min, 57° C. 3 min, 72° C. 1 min, 15 cycles of 98° C. 10 s, 65° C. 15 s, 72° C. 30 s, followed by 72° C. 5 min. Then the PCR products were run on the 2% E-Gel® EX Agarose Gel (Invitrogen), the 250-600 base pair (bp) fragments were isolated and purified using the MINELUTE Gel Extraction Kit (Qiagen). The concentration of the library was measured by Qubit dsDNA HS kit (Thermo Fisher Scientific). The paired-end sequencing was performed on Illumina HiSeq 3000.

### Data Analysis

### Demultiplexing and Data Analysis of iscChIC-seq Libraries

The scripts for de-multiplexing and genome-wide mapping are available at github.com/wailimku/testing123. For profiling each type of histone marks, 30 single cells were sorted into each of the 480 wells by FACS and sent to sequencing after the library's preparation steps. All sequencing data was paired-end. The R2 reads contained the information of cell barcodes, in which the cell barcode sequences followed the common sequence

(SEQ ID NO: 1)
AGAACCATGTCGTCAGTGTCCCCCCCCC.

For each well, R1 reads were mapped to the human reference genome (UCSC hg18) using Bowtie2 (Langmead and Salzberg 2012). Using the cell barcode information from R2 reads, the mapped R1 reads were separated into 96 sets corresponding to the 96 cell barcodes. Reads with mapping quality less than 10 were removed and duplicated reads were removed. For each well, in order to determine the sets of mapped reads among the 96 sets were from single cells, the 96 sets of mapped reads were ranked based on the total number of mapped reads in the sets. A set of reads were considered to be from single cells if they satisfied: 1) They were one of the top 25 ranked sets. 2) The total number of mapped reads in the set was greater than 1000. Note that, using the calculation of collision rate from a previous study(Cusanovich et 404 al. 2015), 25 sets of reads were considered from single cells if 30 single cells were sorted into a well. Thus, the top 25 ranked sets were considered in criterion 1 above. As a result, combining all single cell data from the 480 wells, about 10,000 single cells were identified for both H3K4me3 and H3K27me3.

### Quality Analysis of the Single Cell Data

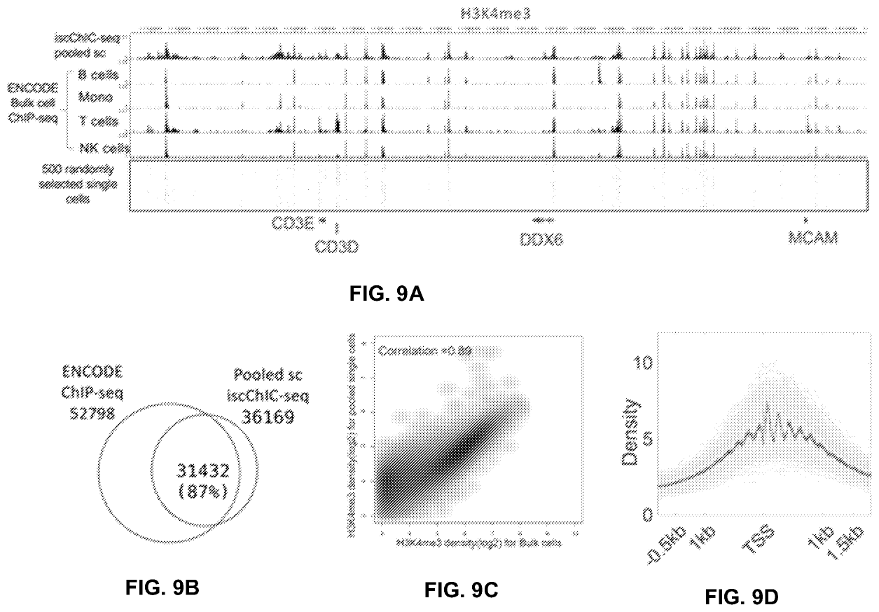

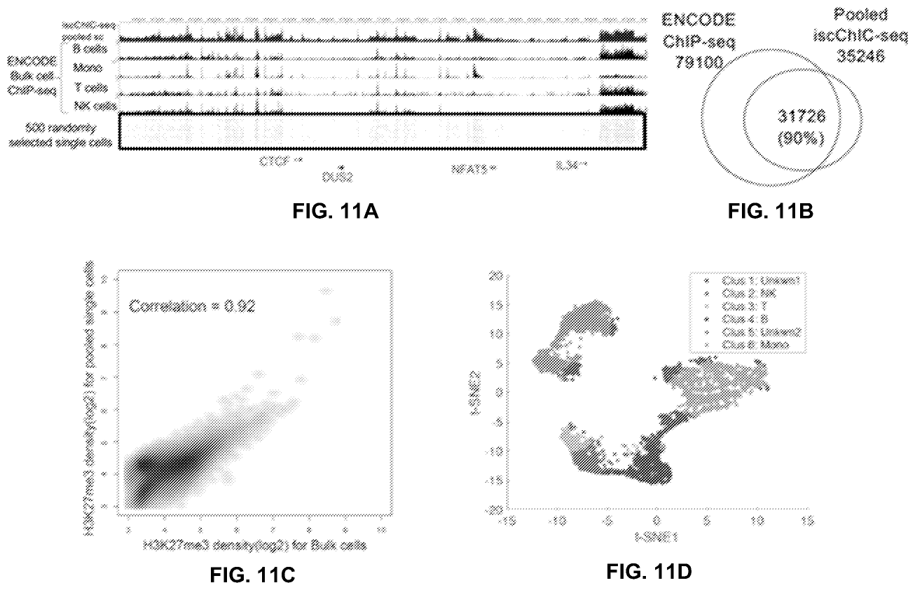

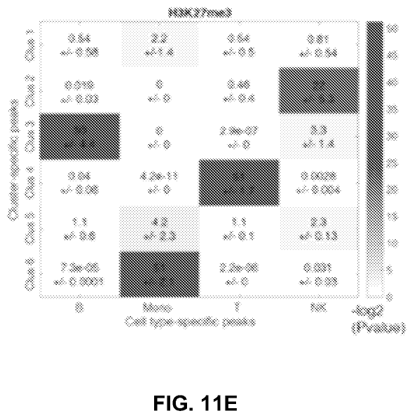

Visualization in Genome Browser. For H3K4me3 and H3K27me3, 2,000 single cells were randomly selected and pooled together as the pseudo-bulk cell data. This pseudo-bulk cell data was visualized using the WashU genome browser(Zhou X. et al. 2011. The Human Epigenome Browser at Washington University. Nat Methods 8: 989-990) (FIGS. 9A and 11A). For H3K4me3, to compare with a benchmark, the H3K4me3 ChIP-seq data of different human white blood cells types was downloaded from the ENCODE(Kazachenka et al. 2018) project shown in the genome browser (FIG. 9A). For H3K27me3, to compare with a benchmark, the H3K27me3 ChIP-seq data of different human white blood cells types was also downloaded from the ENCODE project and visualized in the genome browser (FIG. 11A).

Peaks calling. To examine the quality of the single cell data, the pooled single cell data were compared to the bulk cell ChIP-seq data downloaded from ENCODE (Kazachenka A. et al. 2018. Identification, Characterization, and Heritability of Murine Metastable Epialleles: Implications for Non-genetic Inheritance. Cell 175: 1717). Peaks of this ENCODE data were called using SICER (Zang C. et al. 2009. A clustering approach for identification of enriched domains from histone modification ChIP-Seq data. Bioinformatics 25: 1952-1958; Xu S. et al. 2014. Spatial clustering for identification of ChIP-enriched regions (SICER) to map regions of histone methylation patterns in embryonic stem cells. Methods Mol Biol 1150: 97-111). A final set of peaks for each histone marks was obtained by combining the peaks from different immune cell types. Totally, the final combined sets of peaks obtained from ENCODE data contained 52,798 and 79,100 peaks for H3K4me3 and H3K27me3, respectively. Peaks from the pooled single cells were identified using SICER and their widths were fixed to be 3,000 and 10,000 for H3K4me3 and H3K27me3, respectively. The overlap between peaks from the pooled single cells and the bulk-cell data were computed using the function “findOverlaps” in the R packages “GenomicRange”(Lawrence M. et al. 2013. PLOS Comput Biol 9: e1003118.).

Scatter plots. The human genome was equally divided into bins (bin size=5 kb for H3K4me3; bin size =50 kb for H3K27me3). For both bulk cell and pooled single cell libraries, the read density (counts per million, CPM) at each bin was calculated. The correlation between the logarithm of the read densities of two libraries was quantified using the Pearson correlation coefficient (FIGS. 9C and 11C).

TSS profile plots. For H3K4me3, the software Homer(Heinz et al. 2010) was used to calculate the TSS density profile (annotatePeaks.pl tss mm9 -size 3000 -hist 20 -len 1) for each single cells. In particular, a region of 3 kb around each TSS was considered. This region was then divided into 150 bins. The density profile was generated using the number of reads mapped onto the bin divided by the total number of mapped reads, and averaged over all promoters.

### Clustering Analysis for the iscChIC-seq Data

Expression matrix. Single cells with reads more than 3000 (4000) were first selected. This resulted in 7798 and 9207 single cells for H3K4me3 and H3K27me3, respectively. Second, it was required that the fraction of reads in peaks higher than 0.15 (0.15) were selected for clustering analysis for H3K4me3 (H3K27me3) single cell data. This resulted in 6,021 and 7,038 single cells for H3K4me3 and H3K27me3, respectively. For each cell in H3K4me3 (H3K27me3), reads located within the 52,978 (79,100) combined H3K4me3 (H3K227me3) were counted. A consensus clustering approach was applied, that is similar to SC3 (Kiselev et al. 2017), to the iscChIC-seq data. First, a read count matrix R was computed, in which the columns correspond to cells and rows correspond to the peaks. Rᵢⱼ indicates the number of reads at the ith peak from the jth cell. Each column in the read count matrix was divided by the library size and multiplied by a factor of 10⁶. The resulting matrix denoted as M. The log 2 transformation was further applied resulting M′ where M′=log₂(M +1). For filtering the non-informative bins, a binary matrix Mb was obtained from M′ and defined as,

$${M}_{ij}^{b} = \{ \begin{matrix}0 & if {M}^{′} ij \leq 0 , \\ 1 & if {M}^{′} ij > 0.\end{matrix}$$

The ith row (peak) in the matrix M′ would be selected if

$${\sum }_{j = 1}^{total \# of cell} {M}_{ij}^{b} < {C}_{p e a k} , where {C}_{p e a k}$$

value equals to 100 for both H3K4me3 and H3K27me3, respectively. The filtering of these bins is based on the assumption that reads at a bin should be found in more single cells if the bin is more informative. The expression matrix was denoted after the deletion of rows (peaks) as M″.

Calculation of the Laplacian matrix. Consider mj to be a vector equal to the jth column (cells) of M″. First, the similarity between cells was computed using the Pearson correlation, and resulting a correlation matrix C. In particular, Cij is the Pearson correlation value between the vectors mj and mi. Thus, the rows and columns of the matrix C correspond to single cells. The Laplacian matrix L is defined by L=I−D^{-1/2}AD^{-1/2}, where I is the identity matrix. A is a similarity matrix where A=e^{-(2-C)/max(2-C). Note that D is the degree matrix of A, a diagonal matrix that contains the row-sums of A on the diagonal (D}ᵢᵢ=ΣᵢAij). The eigenvectors of the Laplacian matrix were computed and formed a matrix V where each column represents an eigenvector. The columns of V from left to right are sorted in ascending order based on their corresponding eigenvalues.

Optimal number of clusters. The silhouette analysis was applied to determine the optimal number of clusters. First, a matrix W^{S1} was created, which is a submatrix of V and Wij^{S1}=Vᵢⱼ. Note that i is from 1 to the total number of bins and j=1, . . . s₁. s₁ is fixed to be 12 for both H3K4me3 and H3K27me3. The K-mean method was applied to the matrix W^{S1} el for clustering single cells into k clusters and computed the silhouette coefficient for the clusters. By varying the number of clusters k from 4 to 12, the optimal k value was determined by selecting the case of k having the largest silhouette coefficient value. The optimal k is equal to six for both H3K4me3 and H3K27me3.

Clustering. A binary matrix E was considered in which its rows and columns correspond to single cells. The K-mean method was applied to the matrix W^{t} to cluster the single cells with k=6. If cells i and j belong to the same cluster, Eij=Eji=1; otherwise 0. We consider t is between 2 to 15 and for each t, the clustering analysis was repeated for 10 times and thus obtaining 10 different—E^{s}. A final matrix E^{c} is calculated by averaging all binary matrices from each individual clustering.

t-SNE visualization. The dimension reduction method t-SNE was applied to the matrix E^{c}. The position of single cells is visualized in the two-dimensional t-SNE representative space.

### Clusters Annotation for Both H3K4me3 and H3K27me3

Cluster annotations. After clustering single cells from the single cell H3K4me3 or H3K27me3 data, the clusters were annotated to cell types using the bulk cell ENCODE data. First, the H3K4me3 and H3K27me3 ENCODE data was downloaded for B cells, monocytes, T cells, and NK cells. There were at least two replicates for each histone marks and each cell type. For both H3K4me3 and H3K27me3, the density matrices with log 2 transformation (V^{B}, V^{mono}, V^{T}, V^{NK}), which was similar to M″, were computed for the four cell types, respectively. The number of rows was equal to the number of peaks while the number of columns was equal to the number of replicates. Note that peaks that were deleted in the single cell analysis were also deleted for the bulk cell density vectors. The student t-test was used to compute the cell-type specific peaks from the four density matrices (V^{B}, V^{mono}, V^{T}, V^{NK}). The ith row vector of the matrix V^{z} (Z=B, mono, T, or NK) was denoted as vₗ^{z}. The ith peak (row) was specific to a cell type Z if Vᵢ^{z} is significantly higher than all v_{l′}^{Y} with a p-value of 0.05 and mean(v_{i′}^{z})−mean(vᵢ^{Y})>a cutoff (0.4 for H3K27me3, and 0.2 for H3K4me3), where Y=B, mono, T, NK and Y≠Z. For the purpose of cluster annotation, the sets of cell-type-specific peaks (specific to cell type Z) were denoted as S_{4,an,z} and S_{27,an,z} for the H3K4me3 and H3K27me3 bulk cell data, respectively.

For each histone mark, pseudo-bulk log 2 density matrices (W¹, W², W³, W⁴, W⁵, W⁶) were computed for cluster 1, 2, 3, 4, 5, and 6, respectively. In each of these matrices, the number of columns was equal to the number of peaks while the number of rows was equal to the number of pseudo-bulk replicates. To generate Wⁱ (i=1, 2, 3, 4, 5, 6), six sub-samples of cells were randomly selected from the cells belonging to cluster i, in which the size of each subsample was equal to one-third of the number of cells belonging to cluster i. By pooling the cells in each sub-sample, the log 2 density for each peak was calculated for obtaining Wⁱ. The jth row of Wⁱ was denoted as Wⱼⁱ. The jth peak was specific to a cluster i if Wⱼⁱ was significantly higher than all Wⱼ^{k} where k=1,2,3,4,5,6 and k≠i. Note that p-value computed by student-t test was required to be smaller than 0.05 and mean (Wⱼⁱ)-mean (Wⱼ^{k}) was higher than a cutoff (0.1 for both H3K4me3 and H3K27me3). The sets of cluster-specific peaks (specific to cluster i) for the use of cluster annotation were denoted as X_{4,an,i} and (X_{27,an,i} for the H3K4me3 and H3K27me3 bulk cell data, respectively.

The set of cluster-specific peaks and cell-type-specific peaks were compared. For H3K4me3 data, the p-value for the intersect between a cell type Z and a cluster i (X_{4,an,i}∩S_{4,an,z}) was computed by the hypergeometric test. A cluster i was considered to be annotated validly to a cell type Z if the p-value for (X_{4,an,i}∩S_{4,an,z}) is smaller than 11e -05 and the p-value for other comparisons (X_{4,an,i}∩S_{4,an,z}) Y=B, mono, T, NK but ≠Z) is greater than 1-05.

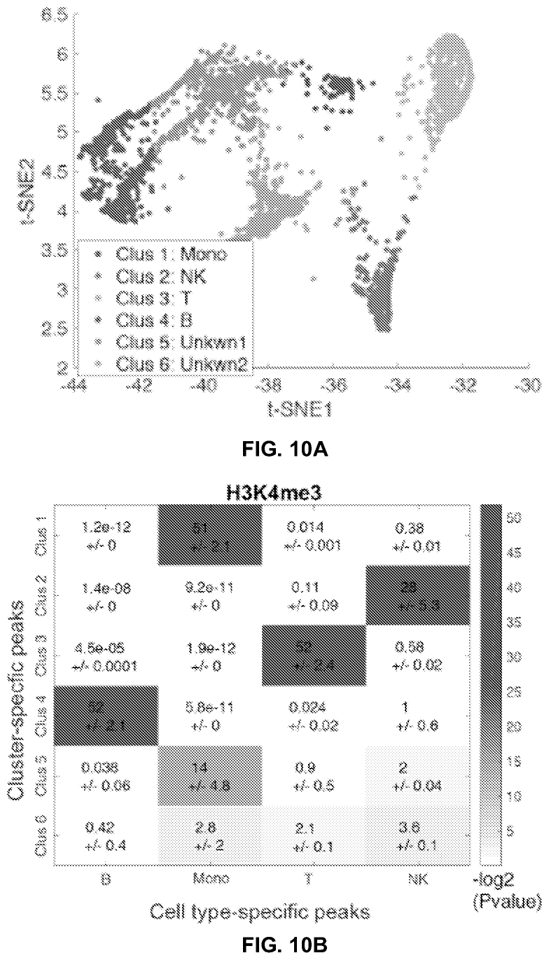

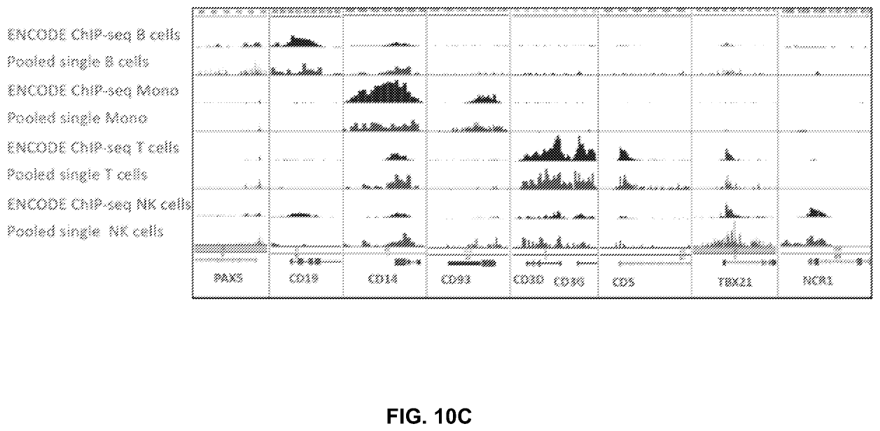

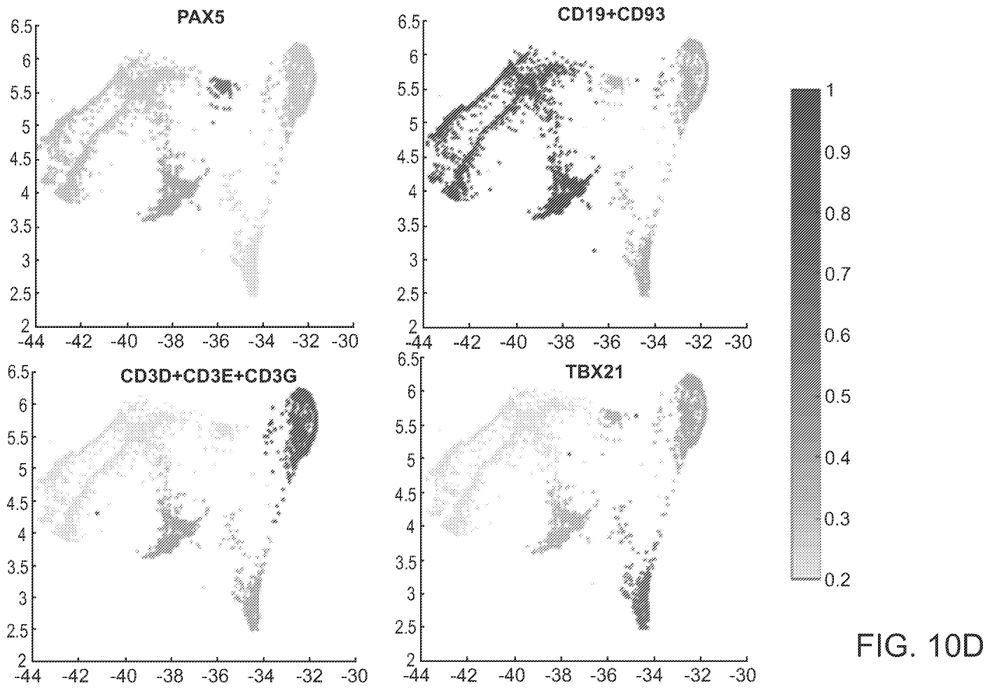

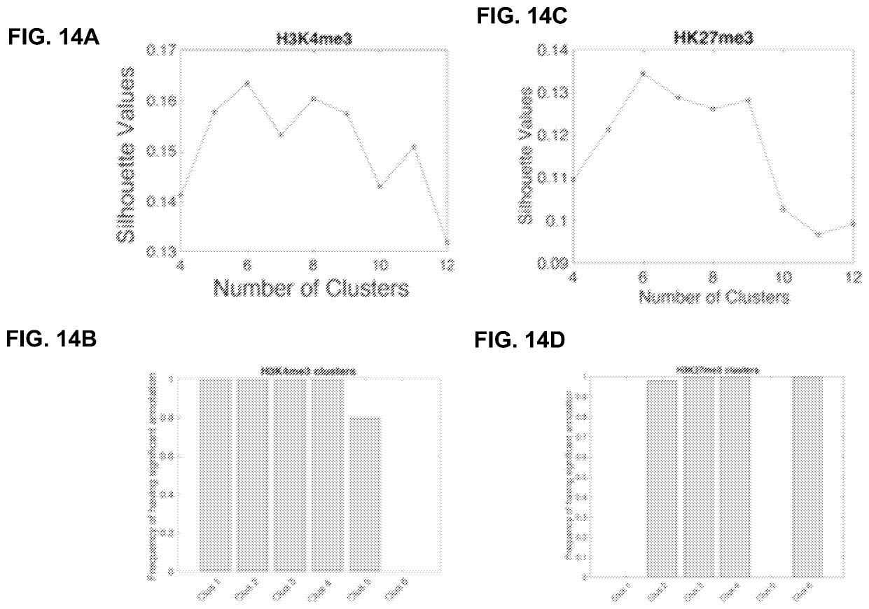

Reproducibility of cluster annotations. To check how reproducible the cluster annotations is, the computations were for 100 times and the cluster density matrices were re-generated each time via the same sub-sampling procedures. The mean and the standard deviation of the p-value in the comparisons were computed and shown in FIGS. 10B and 11E. Also, the frequency for a cluster to obtain a valid annotation was recorded and shown in FIGS. 14B and 14D. To consider a cluster annotation is valid finally, we required that the frequency for a cluster to obtaining a valid annotation is greater than 0.9.

Matching the clusters between H3K4me3 and H3K27me3 marks. For either single cell H3K4me3 or H3K27me3 data, six clusters were found where four of them were annotated as monocytes s T cells, B cells, and NK cells, respectively. If a cluster obtained from single cell H3K4me3 data annotated with a cell type, this cluster was expected to correlate with the cluster obtained from single cell H3K27me3 data annotated with the same cell type.

Bivalent domains were defined as regions where H3K4me3 and H3K27me3 peaks obtained from ENCODE data that were overlapped (command: bedtools intersect-a ‘113K27me3 peak file’ -b ‘113K4me3 peak file’). 25,951 bivalent domains were obtained, in which 7,989 bivalent domains were overlapped with the TSS regions. For both single cell H3K4me3 and H3K27me3 data, we computed the pseudo-bulk log 2 density (W^{B,4}, W^{mono,4} W^{T,4}, W^{NK,4} and W^{B,27}, W^{mono,27}, W^{T,27}, W^{NK,27}) for clusters annotated to B cells, Monocytes, T cells and NK cells, respectively. To generate W^{z,4} or W^{z,27}, six sub-samples of cells were randomly selected from the cells belonging to cluster annotated to cell type Z, in which the size of each subsample was equal to two-third of the number of cells belonging to that cluster. By pooling the cells in each sub-sample, the log 2 density for each peak was calculated for obtaining W^{z,4} or W^{z,27}. The jth row of W^{z,4} was denoted as Wⱼ^{Z,4} while the jth row of W^{z,27} was denoted as W^{2,27}. A peak was specific to a H3K4me3 cluster annotated to cell type Z if Wⱼ^{Z,4} was significantly higher than all Wⱼ^{Y,4} where Y=B, mono, T, NK but Y≠Z. Note that FDR of the p-value (computed by student-t test) was required to be smaller than 0.05 and mean(Wⱼ^{Z,27})-mean (Wⱼ^{Y,4} ) was larger than 0.3. A peak was specific to a H3K27me3 cluster annotated to cell type Z if Wⱼ^{Z,27} was significantly lower than all Wⱼ^{Y,27 where Y=B, mono, T, NK but Y≠Z. Note that FDR for the p-value was required to be smaller than} 0.05 and mean (Wⱼ^{Z,27})-mean(Wⱼ^{Y,27}) was smaller than 0.3. The sets of cluster-specific peaks (specific to cluster annotated to cell type Z) for the use of matching H3K4me3 and H3k27me3 clusters were denoted as X_{4,mat,z} and X_{27,mat,z} for the H3K4me3 and H3K27me3 clusters, respectively. The p-value for the intersection X_{4,mat,z}∩X_{27,mat,z} was computed by hypergeometric test, where Z, Y=B, mono, T, NK.

Relationship between cell-to-cell variation in H3K4me3 and H3K27me3. Different from the procedures of matching the H3K4me3 and H3K27me3 clusters, all bivalent domains were considered. Also, instead of calculating the pseudo-bulk log 2 density matrices, the vectors of coefficients of variation (CV^{B,4}, CV^{mono,4}, CV^{T,4}, CV^{NK,4} and CV^{B,27}, CV^{mono,27}, CV^{T,27}, CV^{NK,27}) were calculated for the H3K4me3 and H3K27eme3 clusters annotated to B cells, Monocytes, T cells and NK cells, respectively. Similar to the single cell log 2 density matrices M″, the log 2 density matrices for single cells in H3K4me3 and H3K27me3 clusters were denoted as (M^{B,4}, M^{mono,4}, M^{T,4}, M^{NK,4} and M^{B,27}, M^{mono,27}, M^{T,27}, M^{NK,27}) referring to H3K4me3 and H3K27me3 clusters annotated to B cells, Monocytes, T cells and NK cells, respectively. Each of these density matrices has the dimensions of the number of bivalent domains multiplied by the number of single cells in the clusters. The vectors of coefficients of variation were computed using these density matrices over the single cells. For the purpose of finding the relationship between cell-to-cell variation in H3K4me3 and H3K27me3, the jth bivalent domain was specific to a H3K4me3 cluster annotated to cell type Z if log₂cvⱼ^{Z,4} is larger than all log₂cv_{J}^{Y,4} than a cutoff (0.2) where Y=B, mono, T, NK and Y≠Z, and the number of non-zero elements in jth row of M^{Z,4} M^{B,4} is larger than 5% of the mean of the number of non-zero elements overall all rows in M^{Z,4}. The second requirement is to only include those relatively more confident CV value for each cluster. The same calculation was applied to obtain the bivalent domains that were specific to a H3K27me3 cluster annotated to cell type Z. The sets of cluster-specific peaks (specific to cluster annotated to cell type Z) for the use of finding the relationship between cell-to-cell variation in H3K4me3 and H3k27me3 were denoted as X_{4,cv,z} and X_{27,cv,z} for the H3K4me3 and H3K27me3 clusters, respectively. By considering the bivalent domains in the set of X_{4,ev,z}∩X_{27,cv}, the spearman correlation between CVZ^{,4} and CV^{Y,27} for and Y, Z=B, mono, T, and NK.

### Results

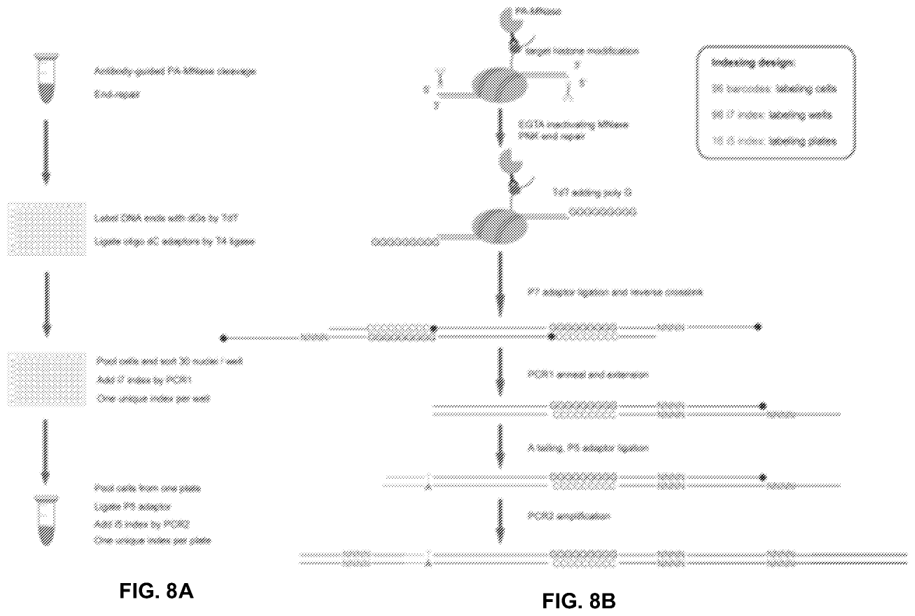

The simultaneous addition of several dG nucleotides to DNA ends by TdT enzyme and ligation of oligo-dC barcode adaptors by T4 DNA ligase is an efficient strategy to barcode chromatin regions following DNase digestion. This barcoding strategy was adapted to label the DNA ends generated by antibody-guided MNase cleavage in ChIC-seq assays to profile histone modifications in more than tens of thousands of single cells in one experiment through three levels of barcoding and indexing strategy (FIGS. 8A, 8B). Briefly, following antibody-guided MNase digestion of cells cross-linked with formaldehyde and disuccinimidyl glutarate (DSG), several dGs were added to the DNA ends by the activity of TdT in the presence of T4 DNA ligase and oligo-dC barcode adaptors in a 96-well plate. The cells were then pooled from 96 wells and aliquoted into new 96-well plates with 30 cells per well by flow cytometry sorting, followed by two consecutive rounds of PCR amplification. The samples were then pooled, purified, and sequenced using Illumina Hiseq3000. The barcodes and PCR indexes were identified and resolved to reveal single cells using a previous strategy (Cusanovich D. A. et al. 2015. Multiplex single cell profiling of chromatin accessibility by combinatorial cellular indexing. Science 348: 910-914.).

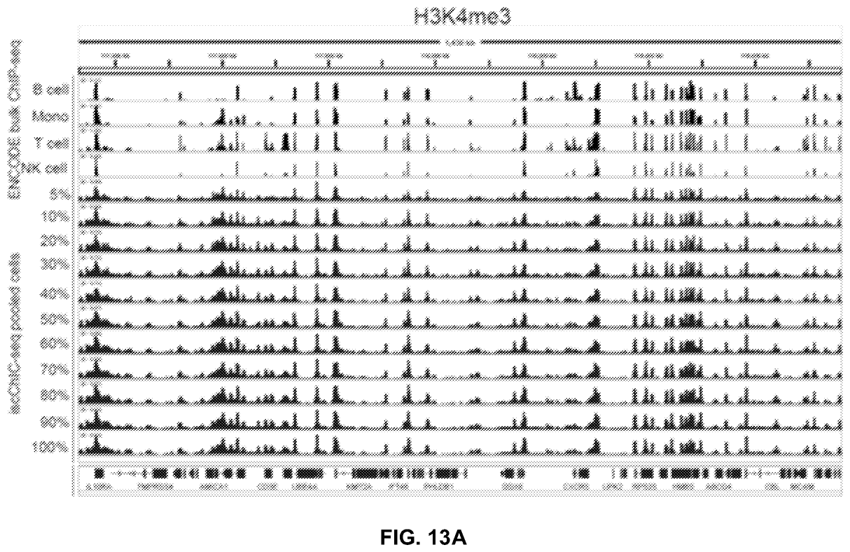

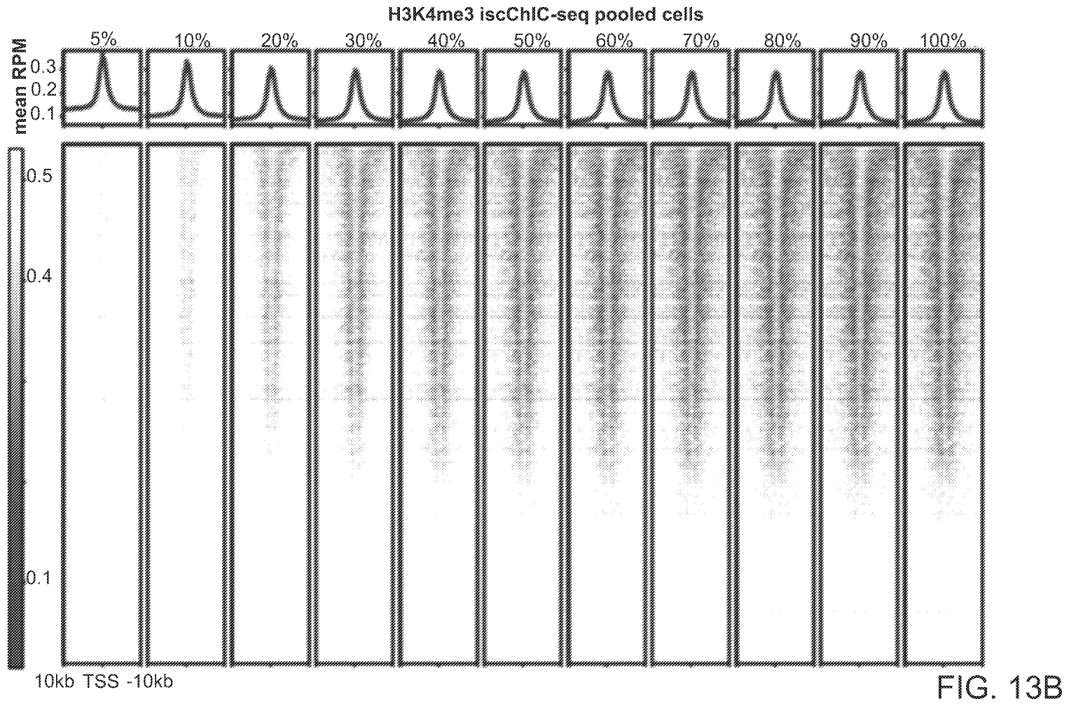

The iscChIC-seq was first applied to white blood cells isolated from human blood for profiling the H3K4me3 modification, which is an active histone modification mark, at a single cell resolution. Using a cutoff to filter cells with less than 1,000 reads, 10,000 single cells and about 9,000 reads per cell on average were detected in one single experiment. Using a more stringent filtering criteria (a cell has at least 3,000 reads), this resulted in ˜7,800 single cells each having about 11,000 reads on average. The cell number and unique reads number per cell detected by iscChlC-seq were significantly improved as compared with the previous published single-cell methods. The genomic profiles of the sequencing read from pooled single cells displayed specific peaks around transcription start site (TSS) and were highly consistent with that of the bulk cell H3K4me3 ChIP-seq data from ENCODE (FIG. 9A and FIGS. 13A, 13B). Using SICER (Zang C. et al. 2009 Bioinformatics 25: 1952-1958; Xu S. et al. 2014. Methods Mol Biol 1150: 97-111), 36,169 H3K4me3 peaks were detected from the pooled single cells. Using a similar strategy, 52,798 H3K4me3 peaks were detected from the ENCODE ChIP-seq data from different immune cells in human WBCs. Comparison of the ENCODE data with the single-cell data revealed that 31,432 out of 36,169 (87%) H3K4me3 peaks from the pooled cells overlapped with the peaks from the bulk cell H3K4me3 ChIP-seq data (FIG. 9B). The read densities of the pooled single cells and the bulk cell ChIP-seq data were highly correlated (r=0.89) (FIG. 9C). Also, the pooled single cell data showed high enrichment and nucleosome phasing around the transcription start site (TSS) (FIG. 9D), as found from ChIP-seq data(Barski et al. 2007). Together, these results indicated that the iscChlC-seq data can effectively detect H3K4me3 marks in single cells.

Next, it was examined if different cell types of the human WBCs, which contain T cells, NK cells, monocytes, and B cells, could be identified from the iscChlC-seq data. For this purpose, a combined reference set of H3K4me3 peaks for human WBCs were first computed using the ENCODE bulk cell H3K4me3 ChIP-seq data (Methods). By applying the silhouette analysis(Rousseeuw P. J. 1987. Silhouettes—a Graphical Aid to the Interpretation and Validation of Cluster-Analysis. J Comput Appl Math 20: 53-65), a number of six were found to be the optimal number of clusters (FIGS. 10A, 14A). To annotate the cells in each cluster, the cells from each cluster were pooled and the H3K4me3 peaks that are specific to each cluster were identified. Using the ENCODE T cell, B cell, NK cell, monocyte bulk cell H3K4me3 ChIP-seq data, the peaks that are specific to each cell type were identified. Next, the statistical significance of the overlap between the two types of specific peaks was calculated using hypergeometric test, which robustly annotated four of the six clusters to be monocytes, T cells, B cells, and NK cells while the other two clusters could not be clearly annotated (FIGS. 10A, 10B). Sub-sampling using 33% of single cells from each cluster confirmed the accurate and reproducible annotation of these cells (FIG. 14B). From the four annotated clusters, 1,610 monocytes, 1,265 T cells, 898 NK cells, and 446 B cells were obtained.

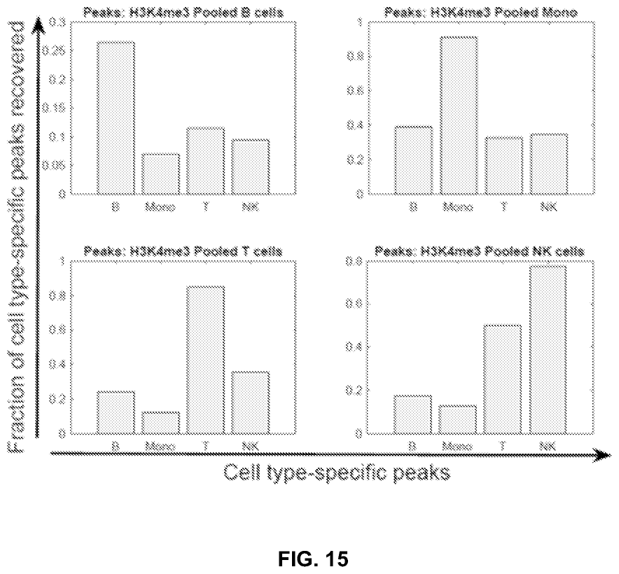

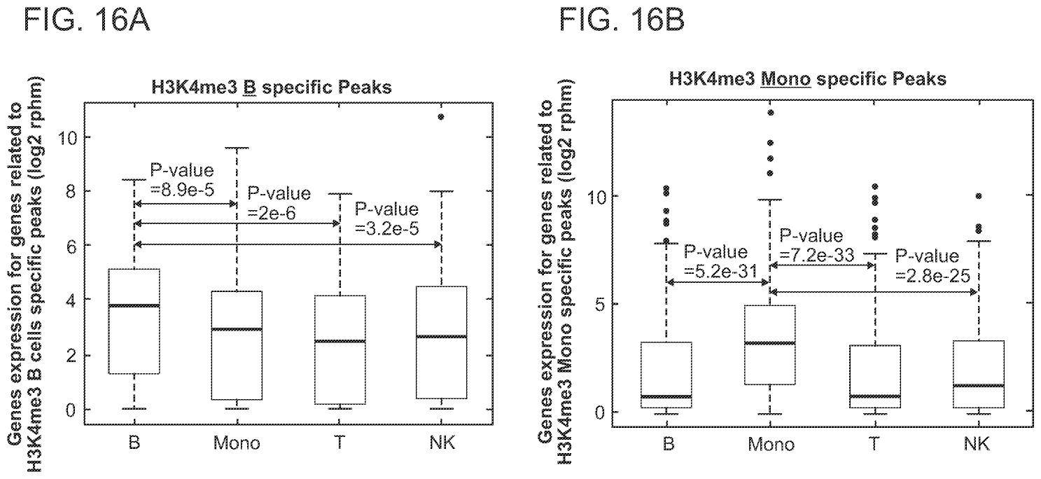

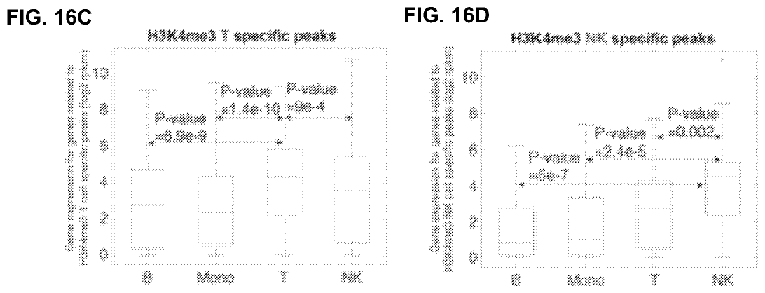

Next, the genomic profiles of the annotated pooled single cell data (from cluster T, B, NK, and monocyte) were compared with the genome profiles of ENCODE bulk cell ChIP-seq data for the corresponding cell types. The analysis revealed that the annotated cluster of single cells showed a genomic profile highly similar to that of the corresponding bulk cells at the cell-type specific gene loci including PAXS, CD19, CD14, CD93, CD3D, CDS, TBX21 and NCR1 (FIG. 31C). By comparing the cell type-specific peaks identified from the ENCODE data and cluster-specific peaks identified from the pooled single cells, it was found that about 80% to 90% of cell type-specific peaks were detected in the pooled single cells from the NK, monocyte and T clusters while only 26% of cell-specific peaks were detected in the pooled single cells from the B cluster (FIG. 15), which may be related to the relatively small number of cells in the B cluster. But, in all cases, much lower fractions of cell type-specific peaks were detected from other cell types than the annotated cell type in the single-cell cluster, indicating the signals from the pooled single cells are specific. Since H3K4me3 is an active mark, the expression levels of genes associated with the specific peaks identified in the pooled single cells from each annotated cluster were compared. The analysis indicated that the genes associated with cluster-specific peaks were expressed at significantly higher levels in the annotated cell type than the other cell types (FIGS. 16A-16D).

At the single cell level, the majority of cells annotated as T cells, B cells, NK cells, monocytes exhibited high H3K4me3 density in regions associated with CD3D+CD3E+CD3G (T cell-specific), PAX5 (B cell-specific), TBX21 (NK and T cell-specific), CD14+CD93 (monocyte-specific), respectively (FIG. 10D). Overall, these results indicate that iscChlC-seq could reliably identify different cell types from a complex population of cells such as WBCs.

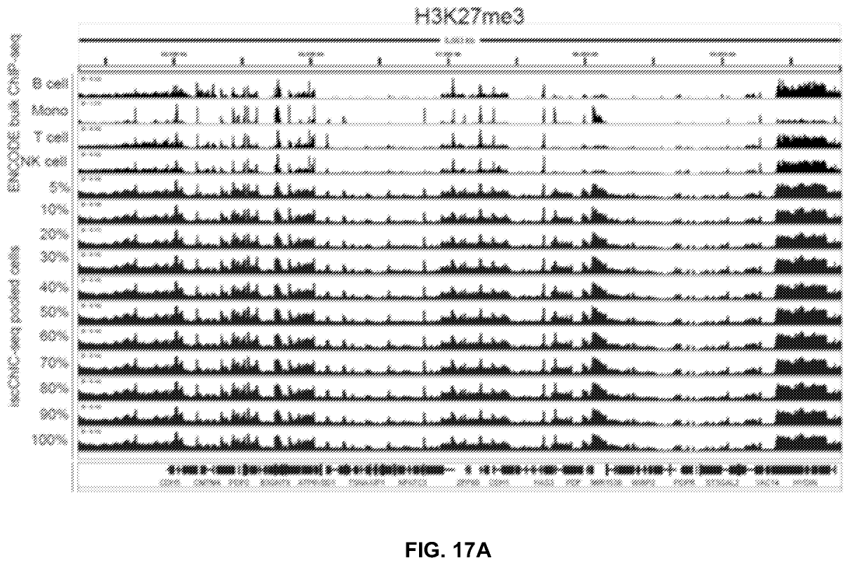

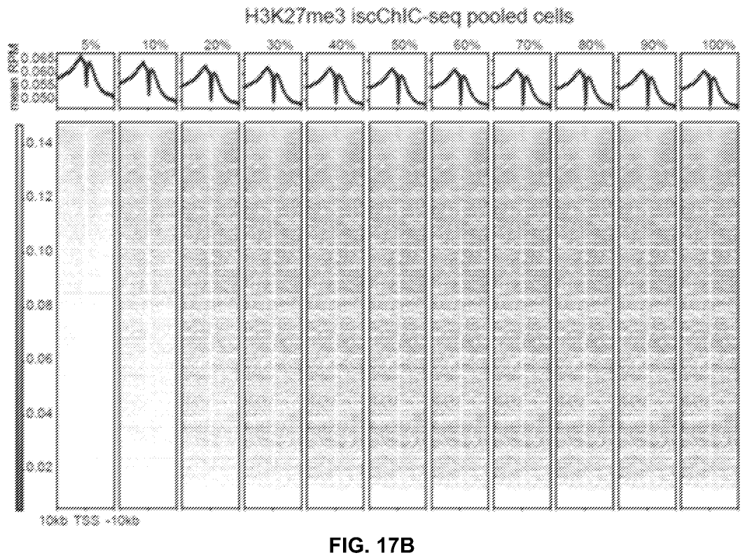

To test if iscChlC-seq worked for detecting repressive histone marks, it was applied to profiling H3K27me3 in WBCs. Using a filtering approach similar to that used for H3K4me3 iscChIC-seq libraries, 10,000 single cells each having about 40,000 unique reads on average were detected. Using a more stringent filtering criteria such that a cell has at least 4,000 unique reads, it resulted in ˜9,000 single cells each having about 45,000 reads on average. The genomic profiles of the pooled single cells were highly consistent with the profiles of the bulk cell H3K27me3 ChIP-seq data from ENCODE (FIGS. 16A, 17A and 17B). A total of 79,110 and 35,246 enriched regions were detected from the ENCODE bulk cell ChIP-seq data and the pooled single cell data, respectively. Comparison of the ENCODE data with the single-cell data revealed that 31,726 of 35,246 (90%) H3K27me3 peaks from the pooled single-cells overlapped with the peaks from the ENCODE H3K27me3 ChIP-seq data (FIG. 11B). The read densities of the pooled single cells and the bulk cell ChIP-seq data were highly correlated (r=0.92) (FIG. 11C). Applying the silhouette analysis to H3K27me3 iscChlC-seq data, an optimal number of clusters equal to six was found (FIG. 14B), which was the same as the H3K4me3 iscChlC-seq data. Similar to the H3K4me3 data, the clustering analysis of the H3K27me3 iscChlC-seq data revealed six clusters of cells (FIG. 11D). After pooling the cells from each cluster, the cluster-specific peaks were identified and compared to the T cell, B cell, NK cell, monocyte specific peaks identified from the ENCODE bulk cell ChIP-seq data. Four cell clusters, including 1,146 T cells, 432 B cells, 749 NK cells, 2,192 monocytes, were annotated by the significant overlap between the two types of peaks (FIG. 11E). Overall, these results indicate that iscChlC-seq could also reliably profile repressive histone marks in a mixed population of cells.

Different from ChlP-seq, ChIC-seq depends on antibody-guided cleavage of chromatin by MNase and thus may have bias toward open chromatin regions. To address this question, all the DHSs were identified from the ENCODE DNase-seq datasets from T, B, NK and monocyte cells and the fraction of the ENCODE bulk cell H3K4me3 ChIP-seq reads that overlapped with DHSs in each cell type were analyzed. The analysis revealed that about 60% to 67% of H3K4me3 CHIP-seq reads from the ENCODE bulk cell H3K4me3 ChIP-seq libraries fell into the DHS regions. In contrast, about 52% to 56% of the H3K4me3 reads from the pooled single cells fell into the DHS regions, providing evidence that the specificity of the H3K4me3 reads from the iscChIC-seq libraries is slightly lower than that of the bulk cell ChIP-seq libraries, which may be caused by differences in washing conditions and/or differences in cell numbers used for the experiments. The H3K27me3 data was also similarly analyzed. These results indicate that while about 38% to 53% of H3K27me3 reads from the ENCODE bulk cell H3K27me3 ChIP-seq libraries fell into the DHS regions, about 33% to 41% of the H3K27me3 reads from the pooled single cells fell into the DHS regions. Thus the percentage of the H3K27me3 reads from the iscChIC-seq libraries in DHS regions is slightly lower than that from the bulk cell libraries, indicating that the H3K27me3 reads detected by iscChlC-seq are not substantially biased toward open chromatin regions. To further estimate the true positive and false positive rates of the iscChlC-seq reads, it was assumed that the peaks from pooled single cells that overlap with those from ENCODE data are true positives while the peaks not overlapping with the ENCODE peaks are false positives. The analysis revealed that while the false positive rate ranges from 1.6 to 2.7%, the true positive rate is about 22% to 32% for H3K4me3 and H3K27me3, respectively.

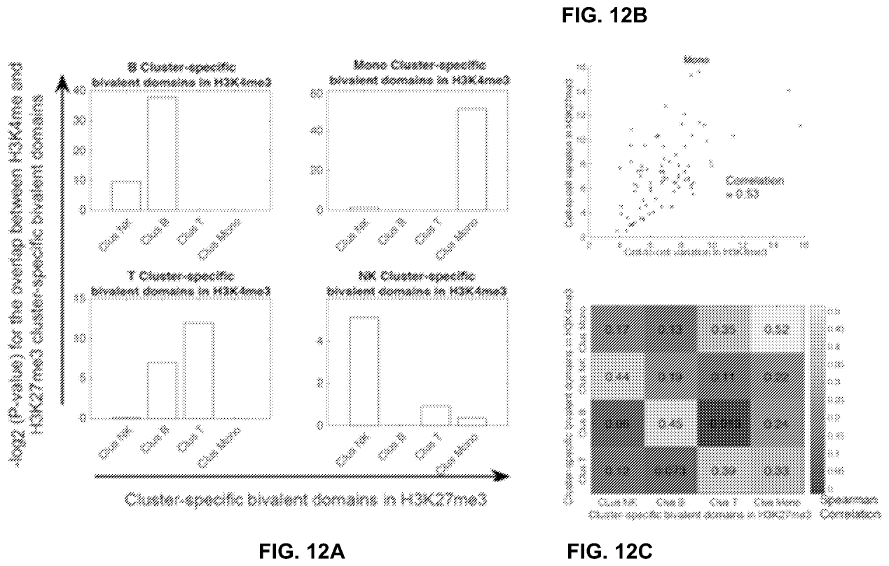

Since the same WBC populations were used in profiling single cell H3K4me3 and single cell H3K27me3, it would be important to examine if a cluster annotated with a cell type from H3K4me3 iscChlC-seq data is specifically correlated with the cluster annotated with the same cell type from H3K27me3 iscChIC-seq data. H3K4me3, an active modification, and H3K27me3, a repressive modification, are co-localized at some key regulatory genomic regions due to either bivalent modifications or cellular heterogeneity (Bernstein B. E. et al. 2006. A bivalent chromatin structure marks key developmental genes in embryonic stem cells. Cell 125: 315-326; Roh T. Y. et al. 2006. The genomic landscape of histone modifications in human T cells. Proc Natl Acad Sci USA 103: 15782-15787; Wang Q. et al. 2019. CoBATCH for High-Throughput Single-Cell Epigenomic Profiling. Mol Cell 76: 206-216 e207; Wei G. et al. 2009. Global mapping of H3K4me3 and H3K27me3 reveals specificity and plasticity in lineage fate determination of differentiating CD4⁺ T cells. Immunity 30: 155-167). The relative levels of these two modifications at these regions are related to each other and influence the expression of underlying genes (Roh et al. 2006). To test this possibility, 7,873 TSS regions (+/−2.5 kb) were first identified which exhibited overlapping H3K4me3 and H3K27me3 peaks from the bulk cell H3K4me3 and H3K27me3 ChIP-seq data in monocytes, T cells, B cells, and NK cells. Next, cluster-specific H3K4me3 peaks among the 7,873 bivalent genes from the H3K4me3 iscChIC-seq data were identified, which are peaks that have higher H3K4me3 methylation level in one cell cluster compared to all other clusters. To relate the H3K4me3 modification with H3K27me3 modification in the iscChlC-seq datasets, it was reasoned that when H3K4me3 level becomes higher, the H3K27me3 level should become lower. Thus, from the four cell clusters based on the H3K27me3 iscChlC-seq data, the cluster-specific peaks among the 7,873 bivalent genes were identified, which are peaks that have lower H3K27me3 methylation level in one cluster compared to all other clusters. Comparison between these two kinds of cluster-specific peaks revealed that the specific peaks of a H3K4me3 cluster is significantly overlapped with the specific peaks of the H3K27me3 cluster if they are annotated as the same cell type (FIG. 12A). These results indicate that the H3K4me3 level is negatively correlated to the H3K27me3 level in the bivalent genes. Further, it was observed that cell-to-cell variation in H3K4me3 and H3K27me3 was positively correlated at bivalent domains in monocytes (FIG. 12B). To match the clusters from single cell H3K4me3 and H3K27me3 data, the correlation analysis was repeated for B cells, NK cells and T cells. Therefore, clusters annotated as B, T, monocyte, and NK from H3K4me3 data were compared with the clusters annotated as B, T, monocyte, and NK from H3K27me3 data. By computing the correlation between the cell-to-cell variation in these clusters, it was found that B, T, monocyte, NK clusters from H3K4me3 data have the highest correlation with B, T, monocyte, NK clusters from H3K27me3 data, respectively (FIG. 12C). The p-value of this observation is 0.0004. This result provided evidence that cell-to-cell variations in H3K4me3 and H3K27me3 are potentially coregulated in the bivalent domains, which can be used to correlate the cell clusters identified from H3K4me3 and H3K27me3 single cell data.

### Discussion

H3K4me3 is usually associated with gene activation, while H3K27me3 is associated with gene repression. The previous single-cell H3K4me3 data indicated that the cell-to-cell variation in H3K4me3 is correlated with the cell-to-cell variation in gene expression (Ku W. L. et al. 2019. Single-cell chromatin immunocleavage sequencing (scChlC-seq) to profile histone modification. Nat Methods 16: 323-325), suggesting that single-cell histone modification data is useful in understanding the cellular heterogeneity in gene expression. However, due to the relatively small number of single-cells (scChIC-seq assay) or relatively sparse unique reads (iACT-seq and scCUT&Tag), the application of these techniques are limited. In this study, the TdT+T4 DNA ligase-mediated barcoding strategy with the scChIC-seq protocol for iscChlC-seq, which enabled the analysis of either active or repressive histone modification profiles in more than 10,000 single cells in one experiment. The assay captured 11,000 unique reads for H3K4me3 or 45,000 reads for H3K27me3 per single cell, which are better than other high throughput techniques for histone modifications. Different from PA-TN5-based techniques, iscChlC-seq works well for both active and repressive marks. Comparison with the bulk cell ChIP-seq data indicated that iscChIC-seq does not have substantial bias toward open chromatin regions for either active or repressive histone modification marks. In addition, iscChlC-seq does not require expensive equipment or special reagents and thus easily accessible to most laboratories with molecular biology capabilities.

The analysis in this study indicated that both the active H3K4me3 and repressive H3K27me3 iscChlC-seq data were effective in clustering the complex WBCs and sorting out different cell types. H3K4me3 and H3K27me3 are colocalized to a subset of genomic regions, which are termed “bivalent domains”. Bivalent modifications are usually associated with key differentiation regulator genes and thus show substantial changes during cell development or differentiation and the expression of a bivalent gene is correlated with the relative level of H3K4me3 and H3K27me3 signals at the gene locus. Although the overlap of H3K4me3 and H3K27me3 peaks at these genomic regions may be caused by different mechanisms including true bivalent modifications and cellular heterogeneity, the dynamic equilibrium of the two opposing modifications at these regions result from the competition of the corresponding enzymes to these regions. Hence, the two functionally opposite modifications may be co-regulated but demonstrate opposite directions. Indeed, the data herein showed that the increased H3K4me3 levels in bivalent genes in one type of cell cluster are positively correlated with the decreased H3K27me3 levels in the same bivalent genes in the same type of cell cluster. The cell-to-cell variations in H3K4me3 and H3K27me3 are positively correlated and exhibit the highest correlation when the cell cluster annotated from the H3K4me3 iscChlC-seq data matches with the same type of cell cluster annotated from the H3K27me3 iscChlC-seq data. Thus, these properties of bivalent modifications can be used to specifically correlate the cell clusters annotated from different single cell H3K4me3 and H3K27me3 data.

Overall, the data herein, show that iscChlC-seq is a reliable single-cell technique for measuring histone modifications and potentially for chromatin binding proteins, which may find broad applications in studying cellular heterogeneity and differentiation status in complex developmental and disease systems.

### Example 3: Multiplex Indexing Approach for the Detection of DNase I Hypersensitive Sites in Single Cells

Cellular heterogeneity in gene expression, has been extensively studied through single-cell sequencing methods. For example, single-cell RNA sequencing (scRNA-seq) has revealed significant heterogeneity in primary glioblastomas (Patel, A. P., et al. (2014) Single-cell RNA-seq highlights intratumoral heterogeneity in primary glioblastoma. Science, 344, 1396-1401). Also, increased levels of heterogeneity in these tumors are inversely correlated with survival, indicating that intratumor heterogeneity should be an essential clinical factor. Successful identification of regulators of this heterogeneity is critical to the development of new therapeutic drugs.

DNase I hypersensitivity of chromatin informs the chromatin states of cis-regulatory elements that govern the expression of target genes including master regulators (Lai, B., et al. (2018) Principles of nucleosome organization revealed by single-cell micrococcal nuclease sequencing. Nature, 562, 281-285. Mezger, A., et al. (2018) High-throughput chromatin accessibility profiling at single-cell resolution. Nat Commun, 9, 3647. Chen, X., et al. (2018) A rapid and robust method for single cell chromatin accessibility profiling. Nat Commun, 9, 5345. Cusanovich, D. A., et al. (2018) A Single-Cell Atlas of In Vivo Mammalian Chromatin Accessibility. Cell, 174, 1309-1324 e1318). Cellular heterogeneity in gene expression has been linked to variation in chromatin accessibility (Jin, W., et al. (2015) Genome-wide detection of DNase I hypersensitive sites in single cells and FFPE tissue samples. Nature, 528, 142-146), nucleosome organization and long distance enhancer-promoter interactions (Jin, W., et al. (2015) Genome-wide detection of DNase I hypersensitive sites in single cells and FFPE tissue samples. Nature, 528, 142-146); thus, measuring chromatin states at the single-cell level is of the utmost importance for understanding the molecular mechanisms of gene expression heterogeneity. Several single cell techniques were developed to measure chromatin accessibility, including scATAC-seq (Buenrostro, J. D., et al. (2015) Single-cell chromatin accessibility reveals principles of regulatory variation. Nature, 523, 486-490. Satpathy, A. T., et al. (2018) Transcript-indexed ATAC-seq for precision immune profiling. Nat Med, 24, 580-590. Lareau, C. A., et al (2019) Droplet-based combinatorial indexing for massive-scale single-cell chromatin accessibility. Nat Biotechnol, 37, 916-924) by Tn5 chromatin tagmentation, scDNase-seq by DNase I digestion for chromatin fragmentation, and scMNase-seq by MNase detection of chromatin accessibility and nucleosome positions. The standard throughput of many of these methods is in the thousands of cells, and of these methods scATAC-seq has the highest cell throughputs; however, it is also known that DNA tagmentation bias exists in the use of Tn5 (Li, Z., et al. (2019) Identification of transcription factor binding sites using ATAC-seq. Genome Biol, 20, 45), which may affect the accuracy of the regulator prediction and cell-to-cell variation in accessibility, limiting its potential applications.

DNase I enzymes have different properties compared to Tn5 (Karabacak Calviello, A., et al. (2019) Reproducible inference of transcription factor footprints in ATAC-seq and DNase-seq datasets using protocol-specific bias modeling. Genome Biol, 20, 42). However, due to a lack of development in combinational indexing strategies for scDNase-seq, its cell throughput is very low and thus its application in single-cell studies is limited. To address this limitation, the study described herein provided a novel indexing strategy, which avoids the use of expensive equipment for automation or microfluidics, to enable the analysis of more than 15,000 cells in a single experiment. This new strategy, termed indexing scDNase-seq (iscDNase-seq), involves barcoding the DNA ends with a combination of TdT terminal transferase and T4 DNA ligase. We applied it to assay single-cell DHSs from human white blood cells (WBC). Computational analysis of the assay results recovered expected cell types from the WBCs and inferred their underlying regulatory mechanisms in accessibility variation. By comparing the iscDNase-seq data obtained herein with publicly available dscATAC-seq data for B cells, T cells, NK cells, and monocytes, it was found that iscDNase-seq detects DHSs missed by scATAC-seq that have high sequence conservation and are associated with significant gene expression. Importantly, iscDNase-seq data can better predict the cellular heterogeneity in gene expression compared to scATAC-seq data. Thus, iscDNase-seq is an attractive alternative method for measuring single-cell chromatin accessibility.

### Materials and Methods

### iscDNase-seq Method

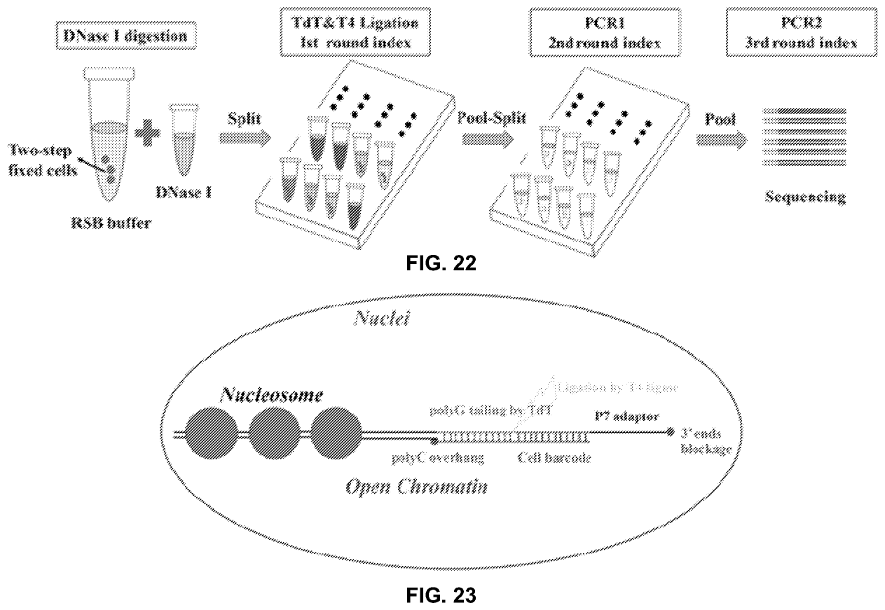

In the iscDNase-seq protocol (FIG. 22), cells were first crosslinked by two-step fixation and subjected to lysis and DNA digestion with DNase I on bulk cells. After removal of DNase I by several washes, bulk nuclei were aliquoted into 96 wells and barcode P7 adaptors were ligated to the chromatin DNA by the TdT&T4 ligation method. The samples were then pooled, diluted, and redistributed to 96 wells of a second plate with 30 nuclei to each well using a flow cytometry sorter. After reverse-crosslinking of DNA overnight at 65° C., a second barcode (well index) primer complementary to the P7 adapter, was introduced to the DNA template directly by one-cycle of polymerase chain reaction (PCR1). Then, all PCR1 products were pooled, ligated to P5 adaptor and re-amplified by PCR2 primers that introduced the third barcode (15 index). Finally, all of PCR2 products were pooled and sequenced, with the expectation that most sequence reads bearing the same combination of barcodes will be derived from a single cell (estimated collision rate of ˜13% for experiments described here)

### Oligonucleotide Sequences

Barcode P7 adaptor top (/5phos/acactgacgacatggttctacaagateggaagagcacacgtctgaactccagtcac/3SpC3/).
Barcode P7 adaptor bottom (tgtagaaccatgtcgtcagtgtcccccccc/3ddC/).
Well index primer (tacggtagcagagacttggtctnnnnnngtgactggagttcagacgtgtgctcttccg).
index primer (aatgatacggcgaccaccgagatctacacacactctttccctacacgacgct).
P7-cs2 primer (caagcagaagacggcatacgagattacggtagcagagacttggtc*t)
P5 adaptor top (/5phos/gatcggaagagcgtcgtgtagggaaagagtg)
P5 adaptor bottom (tctttccctacacgacgctcttccgatct).

### Isolation of PBMC

Human healthy donor bloods were collected and defibrinated or heparinized in a EDTA sodium-treated tubes or bags for anticoagulant of blood by the NIH blood bank. The peripheral blood mononuclear cells (PBMC) were purified by the density centrifugation using Lymphocyte Separation Medium (Corning, catalog no. 45000-726).

Two-Step Crosslinking of Cells

The isolated 50 M of PBMC suspended in 50 ml PBS/MgCl₂ were first fixed by adding 400 μl freshly prepared 0.25 M Disuccinimidyl glutarate (DSG, ThermoFisher Scientific, catalog no.20593) and incubating at room temperature for 45 min with rotation (Tian, B., et al. (2012) Two-Step Cross-linking for Analysis of Protein-Chromatin Interactions. Methods of Molecular Biology, 809, 105-120). After three washes with PBS, the cells were suspended in culture medium DMEM supplemented with 10% FBS and further fixed by adding 1:15 volume of 16% (w/v) methanol-free formaldehyde solution (Thermo Fisher Scientific) and incubating at room temperature for 10 min (Kidder, B. L., et al. (2011) ChIP-Seq: technical considerations for obtaining high-quality data. Nature Immunology, 12, 918-922). The reaction was terminated by adding a 1:10 volume of 1.25 M glycine and incubating at room temperature for 5 min. The fixed cells were collected by centrifugation at 1320 rpm for 7 min and washed with PBS. The fixed cells were stored in aliquots (1×10⁶ cells per tube) at −80° C. until use.

### DNase I Digestion

The two-step fixed cells (1×10⁶) were suspended in 0.5 ml of RSB buffer (10 mM Tris-HCl pH 7.4, 10 mM NaCl, 3 mM MgCl₂, 0.1% Triton X-100) and incubated for 10 min on ice. 50 units of DNase I were added to the cells, followed by incubation in 37° C. water bath for 5 minutes to digest the chromatin (Pilot DNase I titration is needed (Cooper, J., et al. (2017) Genome-wide mapping of DNase I hypersensitive sites in rare cell populations using single-cell DNase sequencing. Nature Protocols, 12, 2342-2354)). The reaction was quenched by adding 10 μl 0.5 M EDTA to a final concentration of 10 mM. The cells were centrifuged at 1320 rpm for 5 mins at 4° C. The supernatants were carefully removed by pipetting without disturbing the cell pellets. The pellets were washed three times using 1 ml 1× T4 ligase buffer (final 0.1% NP40) to remove the DNase I completely.

### TdT and T4 Ligation

The DNase I-digested cells were resuspended in nuclei resuspension buffer (328 μl H₂O; 132 μl 10 mM dGTP; 66 μl 10×T4 ligase buffer; 5.3μl 10% NP40) and equally distributed to 96 wells of a 96-well plate. To add several Gs at the 3′ end of DNA and allow adaptor ligation, 2.5 μl of 10 μM barcode P7 adaptor were added into each well, followed by adding 5 μl of the enzyme dilution buffer (66 μl 10× T4 ligase buffer; 330 μl H₂O; 40 μl TdT enzyme; 13 μl T4 PNK; 78.75 μl T4 ligase) with gentle mixing (pipette up and down 5-7 times). TdT and T4 ligation is performed on the PCR machine for 30 min at 37° C. with lid heating.

### Pool and Split

After TdT and T4 ligation, nuclei were pooled and re-suspended in 1 ml PBS containing 0.1% NP40 and 3 μM DAPI (Invitrogen) for nuclei staining. After 5 min incubation at room temperature, the nuclei were counted under the DAPI fluorescent microscope and 30 nuclei were distributed, using a flow cytometry sorter, into each well of a 96-well plate containing 3 μl reverse-crosslink buffer (50 mM Tris-HCl pH 8.0, 25 ng/ml Proteinase K, 0.1% NP40) mixed with 10 μl PBS containing 0.1% NP40. Up to 6 plates of cells were collected. The plates were sealed completely and incubated at 65° C. overnight on PCR machine with lid heating. After reverse-crosslinking, add 2.5 μl of 2 μM well index primer and 15 μl of 2×PHUSION® master mix (New England BioLabs, catalog no.M0531S) into each well for PCR1 amplification without DNA purification. The PCR1 was done under the following condition: 98° C., 3 min; followed by 12 cycles of 65° C., 30 s and 72° C., 30 s; one cycle of 72° C., 5 min. After PCR1, for each 96-well plate, all of the products were pooled and incubated with 96 μl of Exonuclease I (ThermoFisher Scientific, catalog no. EN0582) at 37° C. for 30 mins to degrade the excessive of well index primers. DNA was then purified by the MINELUTE® Reaction Cleanup Kit (Qiagen, catalog no. 28206).

### Library Preparation and Sequencing

A-tailing and P5 adaptor ligation were performed as described previously (Ku, W. L., et al. (2019) Single-cell chromatin immunocleavage sequencing (scChIC-seq) to profile histone modification. Nature Methods, 16, 323). After P5 adaptor ligation, library DNA is purified by the MINELUTE® Reaction Cleanup Kit. PCR2 was performed by adding 15 μL DNA; 0.4 μl of 10 μM i5 primer; 0.4 μl of 10 μM p7-cs2 primer; 15.8 μl 2×PHUSION® Master Mix with the following condition: 98° C., 3 min; 57° C., 3 min; 72° C., 1 min; followed by 15 cycles of 98° C., 10 s; 65° C., 15 s and 72° C., 30 s; one cycle of 72° C., 5 min. The 220-600 base pair (bp) fragments were isolated using the 2% E-GEL® EX Agarose Gels (Invitrogen, cat #G401002) and purified using the Q1Aquick Gel Extraction kit (Qiagen). The concentration of the purified DNA was measured using Qubit dsDNA HS kit (Thermo Fisher Scientific). The paired-end 50-6-8-50 sequencing was performed using the Illumina MiSeq and HiSeq 3000.

### Data Analysis

### Demultiplexing and Data Analysis of iscDNase-seq Libraries

The scripts for de-multiplexing and genome-wide mapping are available at github.com/wailimku/testing456. 30 single cells were sorted into each of the 480 wells by FACS and sent to sequencing after the library's preparation steps. All sequencing data was paired-end. The R2 reads contained the information of cell barcodes. For each well, R1 reads were mapped to the human reference genome (UCSC hg18) using Bowtie2 (Langmead, B. and Salzberg, S. L. (2012) Fast gapped-read alignment with Bowtie 2. Nat Methods, 9, 357-359). Using the cell barcode information from R2 reads, we separated the mapped R1 reads into 96 sets corresponding to the 96 cell barcodes. Reads with mapping quality less than 10 were removed and duplicated reads were removed. For each well, in order to determine the sets of mapped reads among the 96 sets were from single cells, we ranked the 96 sets of mapped reads based on the total number of mapped reads in the sets. A set of reads were considered to be from single cells if they satisfied:
1) They were one of the top 25 ranked sets.
2) The total number of mapped reads in the set was greater than 1000.

For further filtering the single cells, the merged peaks identified by bulk-cell DNase-seq data were downloaded from ENCODE. Totally, bulk cell DNase-seq libraries were downloaded from ENCODE. For each of the bulk-cell DNase-seq library, peaks were called using MACS2 (Zhang, Y., et al. (2008) Model-based analysis of ChIP-Seq (MACS). Genome Biol, 9, R137), and peaks from all libraries were merged if they overlapped by at least 1 bp. Finally, 218,595 were identified for the bulk-cell DNase-seq data for human WBC. The width of peaks was fixed to be 1,000. A further filtering step was applied to the selected single cells by requiring that reads in single cell need to be more than 4000 and FRiP (fraction of reads in peaks defined by the bulk-cell DNase-seq data) of single cell need to be greater than 0.15.

### Examining the Quality of iscDNase-seq Data

All reads from single cells were pooled together and visualized via the WashU genome browser (Zhou, X., et al. (2011) The Human Epigenome Browser at Washington University. Nat Methods, 8, 989-990) together with the bulk-cell DNase-seq data. Peaks from the pooled single cells were identified using MACS (Zhang, Y., et al. 2008 Genome Biol, 9, R137) and their widths were fixed to be 5,00. The overlap between peaks from the pooled single cells and the bulk-cell data were computed using the function ‘FindOverlap’ in the R package called GenomicRanges (Lawrence, M., et al. (2013) Software for computing and annotating genomic ranges PLOS Comput Biol, 9, e1003118). The read density of pooled single cell and pooled bulk-cell data from the 18 bulk-cell libraries were calculated over the bulk-cell peaks. In particular, peaks with read density equal to 0 from either pooled single cell or bulk cells were removed in the calculation. The correlation between the read densities of pooled single cell and bulk cell was quantified by the Pearson Correlation.

Clustering Analysis for the iscDNase-seq Data

Expression matrix. First, a read count matrix R, was computed in which the columns correspond to cell and rows correspond to DHSs that were identified using pooled single cells. Rij indicates the number reads at the DHS site i from the jth cell. For filtering the non-information DHSs, DHSs with total number of reads over all single cells less than 150 were filtered out.

A Latent Semantic Indexing (LSI) analysis. Similar to the previous studies, latent semantic indexing (LSI) was applied to the read count matrix to reduce the dimensions. To perform the LSI analysis, the read count matrix was normalized by term frequency inverse document frequency (TF-IDF) and then a Singular-Value Decomposition (SVD) was performed on the normalized count matrix (Chen, X., et al. (2018) A rapid and robust method for single cell chromatin accessibility profiling. Nat Commun, 9, 5345; Cusanovich, D. A., et al. (2015) Multiplex single cell profiling of chromatin accessibility by combinatorial cellular indexing. Science, 348, 910-914). By removing the first dimension component after SVD transformation, the inverse SVD transformation was applied, resulting in a normalized read count matrix E′ in which rows correspond to DHSs and columns correspond to cells.

t-SNE visualization and clustering. A t-SNE was applied to the normalized read count matrix E′. The position of single cells was visualized in the two-dimensional t-SNE representative space. Single cells are labeled in two different ways. First, single cells were labeled according to the clusters they were from. Second, single cells were labeled according the annotation of cell types. DB SCAN was applied to the two-dimensional t-SNE representative space for clustering.

Generating Heatmap for the Cluster Specific Reads of iscDNase-seq Data

Identifying cluster specific peaks. The normalized read count matrix E′ was transformed to another normalized matrix G in which rows correspond to DHSs and columns corresponds to clusters. In particular, Gᵢⱼ=mean (E′ ᵢₖ) for all cell k belonging to cluster j. Further, the fold-change of DHSs in each cluster was computed where fold change at peak i for cluster

$$k = min ( \frac{G i k}{Gij} )$$

for all j=1, . . . , 4 and ≠k. For each cluster, DHSs was selected with fold-change greater than 1.5. Finally, the heatmap of E′ at the specific peaks were plotted.

TF motif analysis. For each cluster, AME was applied to the specific peaks for identifying significant motifs, and the top 40 significant motifs were selected first by also requiring p-value <0.01 (McLeay, R. C. and Bailey, T. L. (2010) Motif Enrichment Analysis: a unified framework and an evaluation on ChIP data. BMC Bioinformatics, 11, 165). Then of that set, only motifs exclusive to one cluster were kept.

Comparing iscDNase-seq Against dscATAC-seq

Peak calling. Peaks were identified using MACS calls (parameters:—format bed—nomodel—call-summits—nolambda—keep-dup) on each assay-cell type. Unique peak sets are equivalent to A∩B′ where A is the assay of interest and B is the other assay with both sets belonging to the same cell type of either single cell or bulk assays. Unique intersecting peak sets are equivalent to taking the intersection between two unique peak sets where one belongs to single cells and the other belongs to bulk cells. These set operations are used to yield a refined set of peaks specific to a single cell assay that are also found in the bulk assay with the same digestion enzyme but not in other assays that use different enzymes.

Conservation scores. Unique intersecting peak sets were compared by constructing average conservation score profiles for them. For each peak in a peak set, the average phastCons score was plotted at single bp resolution.

Enrichment analysis. Unique intersecting peak sets were compared by finding the expression of their peaks' nearest genes within 2.5 kbp. Expression data was gathered from GEO and the reads per kilobase per million mapped reads was calculated using rpkmforgenes.py^{24.} Peaks were then annotated using ChIPseeker with the gene expression data from rpkmforgenes.py (Yu, G., et al. (2015) ChlPseeker: an R/Bioconductor package for ChIP peak annotation, comparison and visualization. Bioinformatics, 31, 2382-2383).

### Correlation Between Cell-to-Cell Variation in Gene Expression and Accessibility

Coefficient of variation scores were calculated for peak accessibility and gene expression, where the gene expression data came from 10× Genomics. For annotating peaks with TSS, ChlPseeker ((Yu, G., et al. (2015)) was used with a 20 kbp range, and genes and peaks with no mapped reads were filtered out.

### RESULTS

### TdT Terminal Transferase and T4 DNA Ligase-Mediated Barcoding Strategy

The iscDNase-seq procedure is illustrated in FIGS. 22 and 23. Following DNase I digestion of cells crosslinked with formaldehyde and disuccinimidyl glutarate (DSG), several dGs are added to the DNA ends by the activity of TdT in the presence of T4 DNA ligase and oligo-dC barcode adaptors in a 96-well plate (FIG. 22). Following base-paring with the oligo-dGs at the DNA ends, the oligo-dC barcode adaptors are ligated to the DNA ends by T4 DNA ligase. The cells are then pooled from 96 wells and aliquoted into new 96-well plates with 30 cells per well by flow cytometry sorting followed by two consecutive rounds of PCR amplification and indexing of DHS DNA (FIG. 22). The combination of three rounds of barcoding and indexing enables detection of over 15,000 cells in a single experiment.

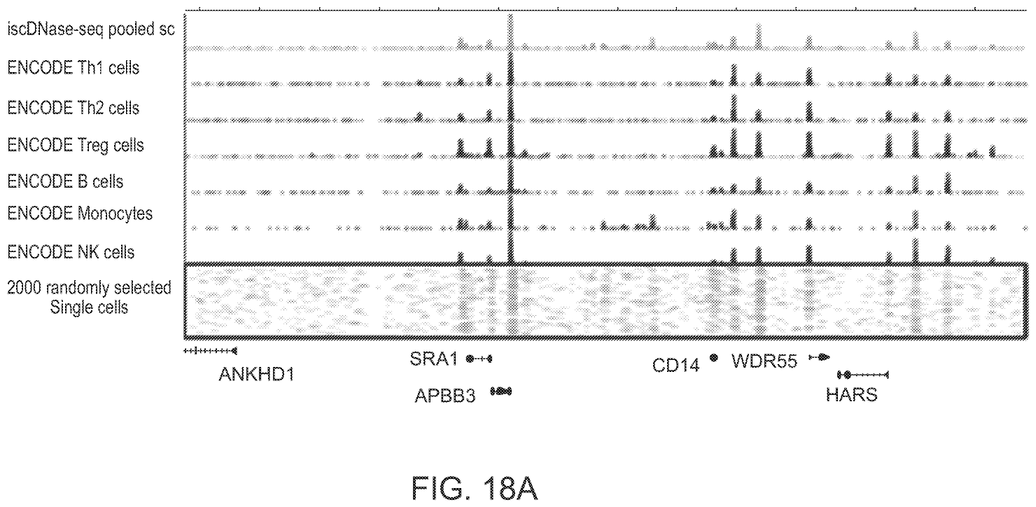

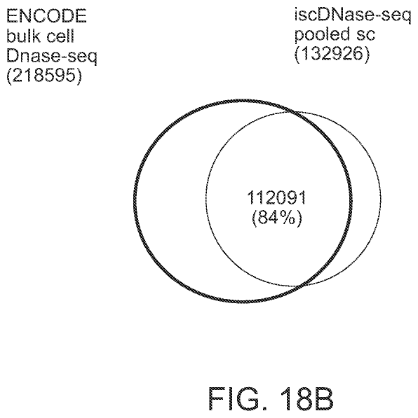

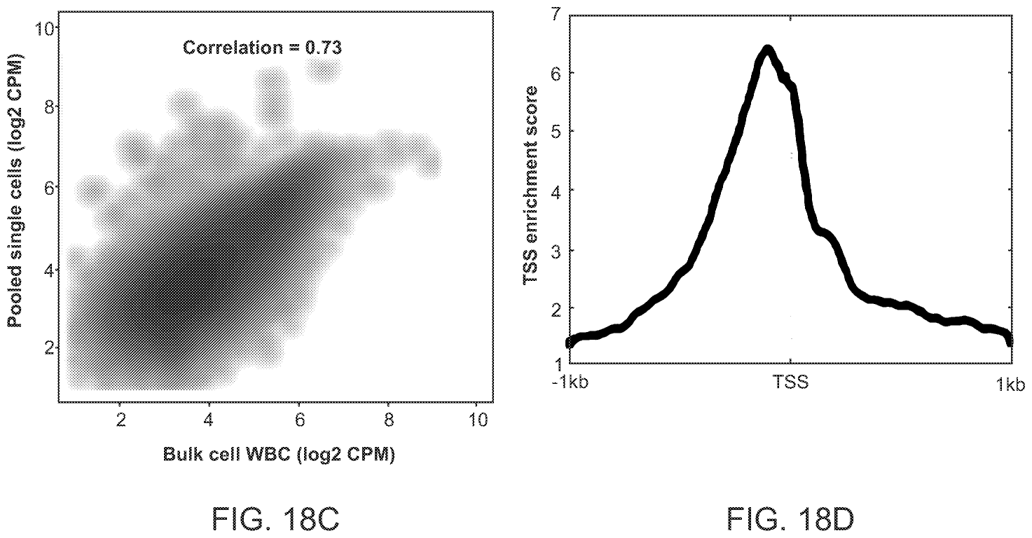

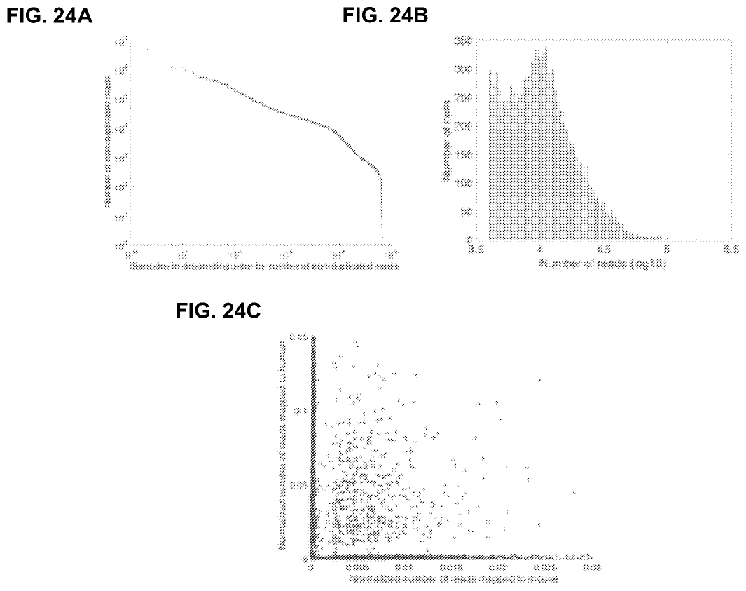

iscDNase-seq was first applied to WBCs purified from human blood to detect open chromatin regions at single cell resolution. Using a cutoff to filter cells with less than 1,000 reads and a fraction of reads in peaks (FRiP) smaller than 15%, d approximately 15,000 single cells and 10,000 reads per cell on average were detected in a single experiment. Using a more stringent filtering criterion where a cell must have at least 4,000 reads resulted in approximately 10,000 single cells and 12,000 reads on average (FIGS. 24A and 24B). To test potential doublet formation by random collision between any two cells, human WBCs and mouse splenocytes mixed, cross-linked, subjected to DNase I digestion and processed for library construction. From the sequencing data, a collision rate of approximately 13% was observed (FIG. 24C), which was similar to a previous barcoding strategy for single-cell ATAC-seq (Cusanovich, D. A., et al. (2015) Multiplex single cell profiling of chromatin accessibility by combinatorial cellular indexing. Science, 348, 910-914). The genome browser snapshots (FIG. 18A) show highly consistent profiles between the pooled single-cell and bulk cell ENCODE DNase-seq data. 218,595 and 132,926 DHSs were detected from the bulk cell ENCODE data and the pooled single cell data, respectively, in which 112,091 (84%) overlapped (FIG. 18B). The read densities of the pooled cells and the ENCODE data were highly correlated (FIG. 18C). Also, the pooled single cell data showed high enrichment around the transcription start site (TSS) (FIG. 18D). All of these results together suggest that the iscDNase-seq method can effectively detect open chromatin regions in WBC.

### iscDNase-seq Data Accurately Cluster Sub-Types of Cells in WBC

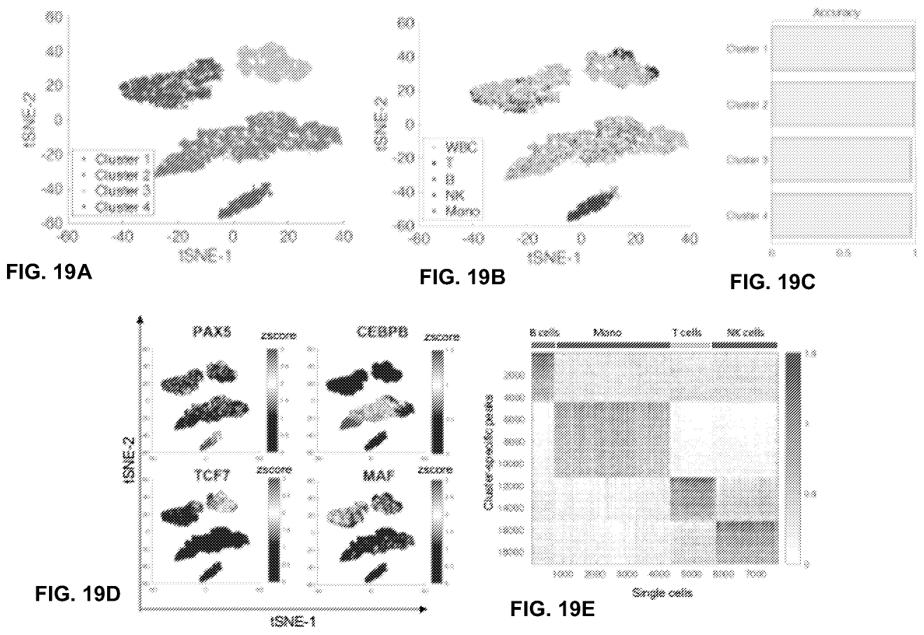

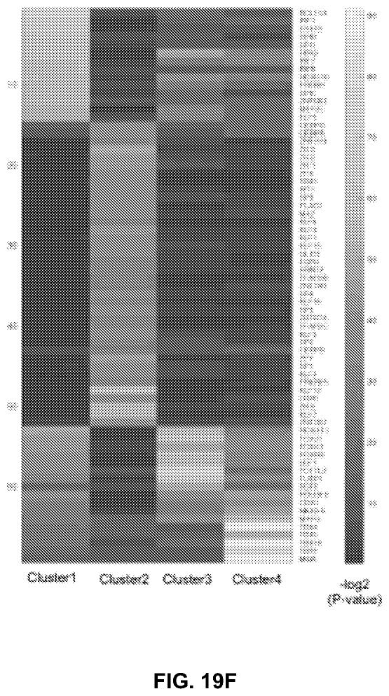

Human WBCs contain T cells, NKcells, monocytes, and B cells. To benchmark cell cluster annotations, iscDNase-seq was applied to human CD4 T cells, B cells, NK cells, and monocytes that were purified by flow cytometry sorting. Using the same filtering strategy as the human WBCs, 699 B cells, 3,590 monocytes, 1,421 T cells, and 1,923 NK cells were obtained. To cluster the single cells from each specific cell type, read counts were first calculated in the DHSs identified from the pooled single cell data for each of the sorted cell types and whole WBCs. Next, the Latent Semantic Indexing method was applied to normalize the data. Finally, the dimensionality reduction t-SNE was directly applied to the normalized read count matrix. Finally, the cluster results were visualized along with annotations of the known cell types and clusters (FIGS. 19A and 19B). The clustering analysis of WBCs revealed four clusters of cells (FIG. 19A). The sorted B cells, T cells, NK cells and Monocytes were clearly clustered separately (FIG. 19B). Comparison between the unsupervised and annotated clusters in FIG. 19B provides evidence that clusters 1, 2, 3 and 4 belonged to B cells, Monocytes, T cells and NK cells, respectively. In order to evaluate the cluster annotations, accuracy was defined as the purity of a cluster or the largest fraction of one of the sorted cell types in a cluster. For example, the fraction of sorted B cells in cluster 1 is close to 100%, while the fractions of other sorted cell types are near zero; thus, cluster 1 cells are more likely to be annotated as B cells, and its cluster accuracy is close to 100%. It was found that the cluster accuracies for clusters 1, 2, 3 and 4, which corresponded to B cells, Monocytes, T cells, and NK cells, were all greater than 97% (FIG. 19C). Within the human WBCs, there were about 47% monocytes, 19% T cells, 25% NK cells, and 9% B cells. Overall, the iscDNase-seq data successfully clustered the four types of immune cells in human WBCs, which indicates that iscDNase-seq is able to identify cell type specific DHSs that can be used in downstream clustering.

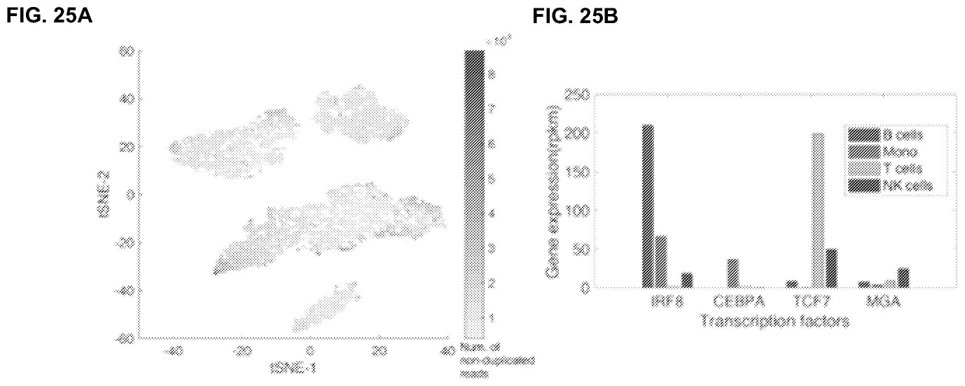

Next, it was examined whether any clusters were results of cell doublet formation. The reads per cell were visualized in the tSNE plots (FIG. 25A), and the results showed that the cells with extremely high read numbers did not aggregate in any one particular cluster, suggesting that the formation of potential doublets did not affect the clustering results. Furthermore, by examining the accessibility of several genes encoding cell-type specific TFs in the cells of the different clusters, we observed that cell-type specific TF genes (PAX5 for B cells, CEBPB for monocytes, TCF7 for T cells, and MAF for NKcells) exhibited the highest accessibility in the clusters annotated to be the same cell types that express the gene (FIG. 19D).

Next, it was examined whether cell type specific regulatory regions could be identified using the iscDNase-seq data. To do this, the marker peaks that can distinguish each cluster from the other clusters were detected. As shown in FIG. 19E, the cluster-specific peaks have the highest normalized read counts in the specifically annotated cell types. To identify potential transcription factors that are associated with the cluster-specific peaks, enriched motifs using AME were detected (Heinz, S., et al. (2010) Simple combinations of lineage-determining transcription factors prime cis-regulatory elements required for macrophage and B cell identities. Mol Cell, 38, 576-589). For each cluster, the top 40 significant motifs were selected first, and then of that set, only motifs exclusive to one cluster were kept (FIG. 19F). It was found that the set of enriched motifs in each cluster included target motifs for specific transcription factors known to be critical to the cell types that the clusters belonged to. For example, the IRF8 motif, which is specific to B cells (Mookerjee-Basu, J. and Kappes, D. J. (2014) New ingredients for brewing CD4⁺ T cells: TCF-1 and LEF-1. Nat Immunol, 15, 593-594), was enriched in cluster 1, which corresponds to B cells; the CEBPA motif, which is specific to Monocytes (Feinberg, M. W., et al. (2007) The Kruppel-like factor KLF4 is a critical regulator of monocyte differentiation. Embo Journal, 26, 4138-4148), was enriched in cluster 2, which corresponds to Monocytes; the TCF7 motif, which is critical to T cells (Simonetta, F., et al. (2016) T-bet and Eomesodermin in NK Cell Development, Maturation, and Function. Front Immunol, 7, 241), was enriched in cluster 3, which corresponds to T cells; and the MGA motif, which is specific to NK cells (Cobaleda, C., et al. (2007) Pax5: the guardian of B cell identity and function. Nat Immunol, 8, 463-470. Wang, H., et al. (2008) IRF8 regulates B-cell lineage specification, commitment, and differentiation. Blood, 112, 4028-4038), was enriched in cluster 4, which corresponds to NK cells. To further confirm whether these TFs were specifically expressed in the corresponding cell types, their gene expression levels in the bulk cell data were examined and the four TFs were found to be specifically expressed in the corresponding cell types (FIG. 25B). These results provide evidence that iscDNase-seq is an efficient method to detect regulatory regions that are associated with cell-type specific TFs.

### iscDNase-seq and scATAC-seq Reveal Both Common and Distinct Information in WBCs

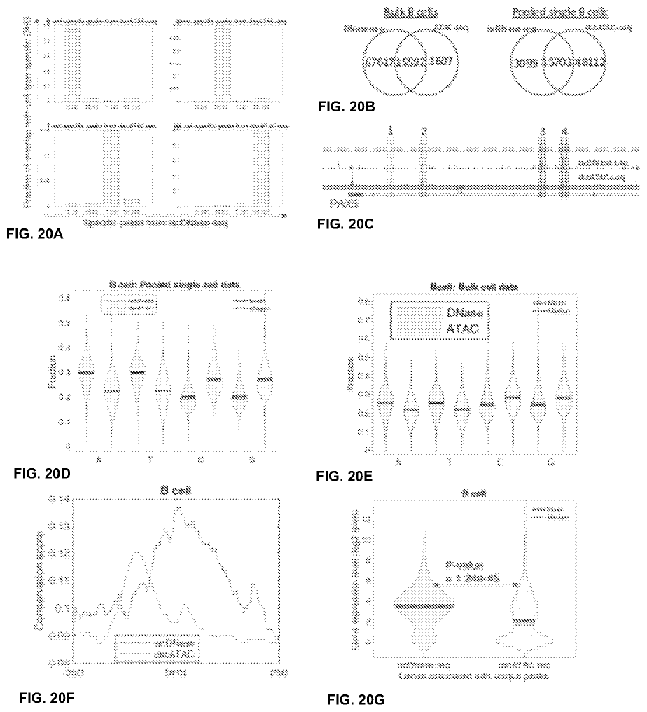

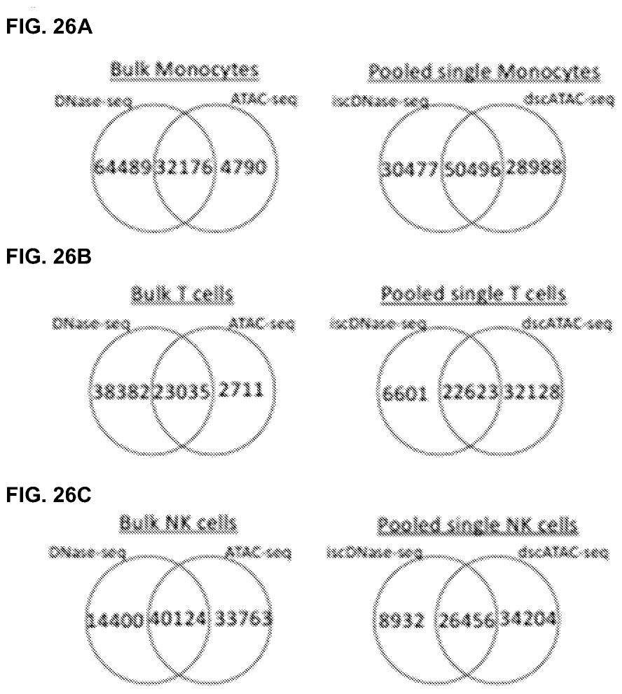

scATAC-seq and iscDNase-seq use different enzymes (Tn5 or DNase I) to probe chromatin accessibility, and thus iscDNase-seq may reveal information that is not recognized by scATAC-seq. To test this idea, the recent single cell ATAC-seq data (dscATAC-seq) for B cells, monocytes, T cells, and NK cells was downloaded (Lareau, C. A., et al (2019) Droplet-based combinatorial indexing for massive-scale single-cell chromatin accessibility. Nat Biotechnol, 37, 916-924). For both dscATAC-seq and iscDNase data, the cell-type specific peaks were identified using MACS with a peak width setting of 500 bp. By comparing the cell-type specific peaks from iscDNase-seq with those from dscATAC-seq, it was found that peaks from iscDNase-seq were highly overlapped with the peaks from dscATAC-seq only when they were from the same cell type (FIG. 20A). This indicates that both assays are able to identify cell-specific open chromatin regions. Global analysis of the accessible sites in single cell and bulk cell assays revealed that a non-trivial fraction of the open regions was detected only by the DNase- or Tn5-related assays (FIGS. 20B, 26A-26C). For example, iscDNase-seq and dscATAC-seq found 3,099 and 48,112 peaks distinct from the other assay in B cells, respectively (FIG. 20B, right panel). Visual inspection of the accessible sites on Genome Browser snapshots revealed distinct sites detected by iscDNase-seq and dscATAC-seq across gene loci. For example, iscDNase-seq and scATAC-seq detected same as well as distinct sites across the PAX5 gene locus in B cells (FIG. 20C). While Site 2 was highly accessible in both assays (brown), Sites 3 and 4 were preferentially detected by iscDNase-seq (red) and Site 1 was preferentially detected by dscATAC-seq (blue).

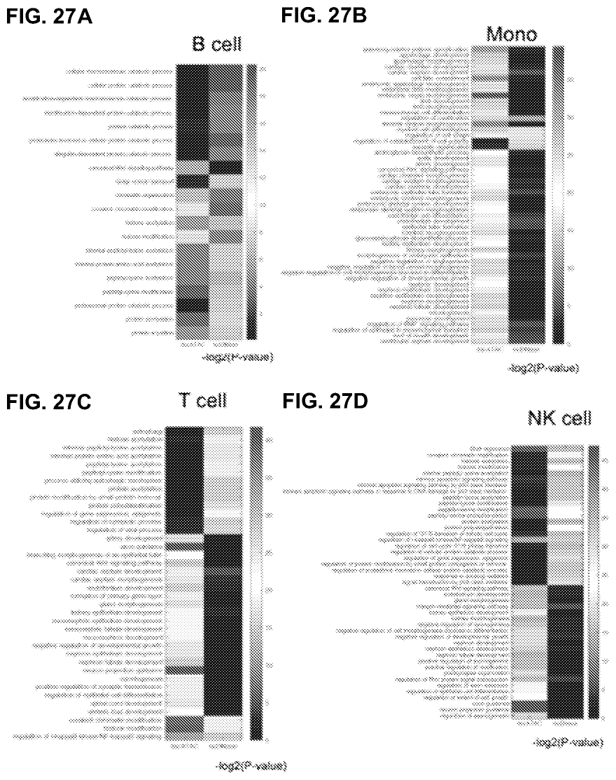

To examine the functional significance of unique sites detected by iscDNase-seq versus dscATAC-seq, the gene ontology terms associated with the unique sites were first analyzed. It was found that the enriched GO terms for the unique sites detected by iscDNase-seq and dscATAC-seq were very different (FIGS. 27A-27D). The GO terms associated with unique iscDNase-seq peaks include histone modifications (B cells), myeloid cell differentiation (Monocytes), chromatin organization and NF-κB signaling (T cells), NF-κB signaling (NK cells). Many of these GO terms are related to immune functions. However, the GO terms associated with unique dscATAC-seq peaks include canonical WTN signaling pathway and kidney epithelium development (B cells), embryonic organ morphogenesis and skeletal system morphogenesis (Monocytes), axon guidance and neuron projection guidance (T cells and NK cells). These terms are not associated with immune functions. From these results, it appears that the unique peaks from the iscDNase-seq datasets are more likely to be associated with cell-specific functions of the underlying cells. Thus, the unique peaks from the iscDNase-seq date sets may be a better predictor of cell-specific enhancers than the unique dscATAC-seq peaks.

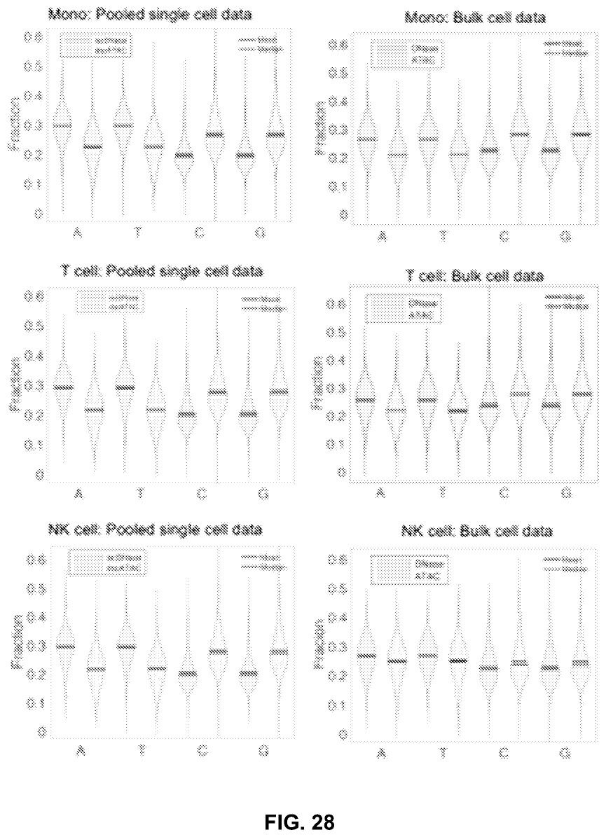

Next, the nucleotide compositions of unique sites detected by iscDNase-seq and dscATAC-seq were compared. It was observed that the unique iscDNase-seq sites were more likely to be AT-rich while the unique dscATAC-seq peaks were more likely to be CG-rich (FIGS. 20D and 28). These trends were also observed in the unique peaks from the bulk cell DNase-seq and ATAC-seq data (FIGS. 20E and 28). It has been suggested that AT-rich regions were more related to the cell type (Vinogradov, A. E. and Anatskaya, O. V. (2017) DNA helix: the importance of being AT-rich. Mamm Genome, 28, 455-464). These results motivated the hypothesis that the unique iscDNase-seq peaks are more likely to contribute to transcriptional regulation than the unique dscATAC-seq peaks do.

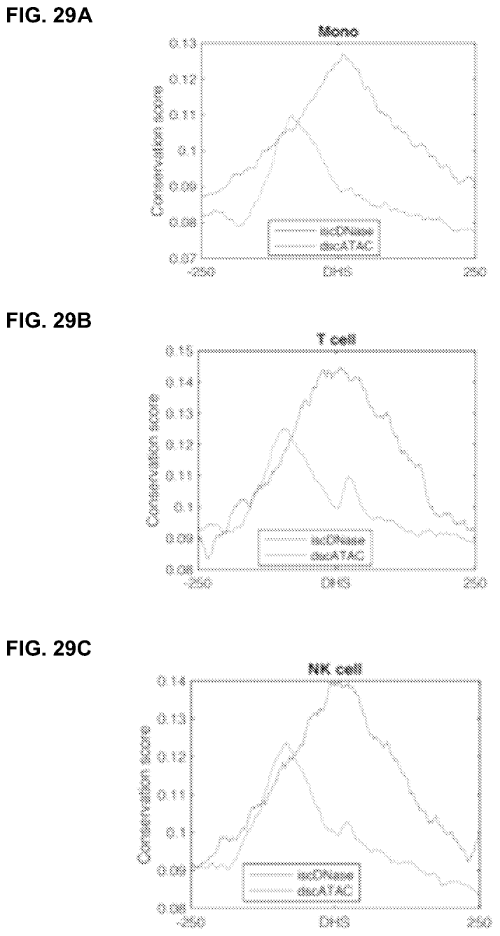

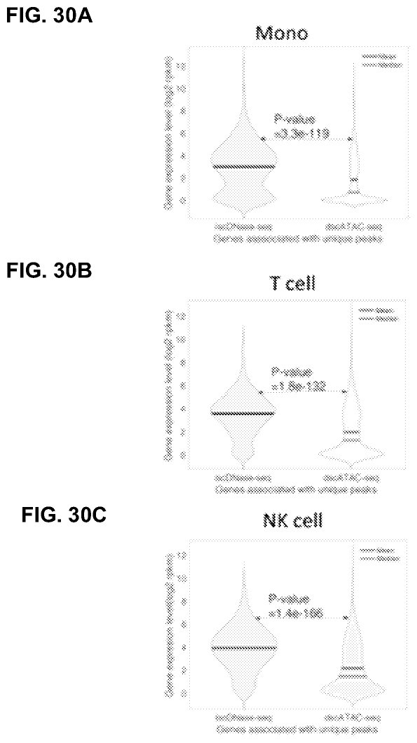

To test this hypothesis, the level of sequence conservation as sequence conservation is often an indicator of functional element was compared. By retrieving the average phastCons conservation scores (31) of the unique iscDNase-seq and dscATAC-seq sites, we observed that the unique DNase-seq sites were more likely to have a conserved region around the center of the sites, while the unique dscATAC-seq peaks have a lower conserved region away from the center of the sites (FIGS. 20F and 29A-29C). Next, the genes that are located near either a unique iscDNase-seq peak or a unique dscATAC-seq peak were identified and the expression levels of the two gene groups was compared. The analysis revealed that the genes located near unique iscDNase-seq sites showed significantly higher expression levels than those located near unique dscATAC-seq sites (FIGS. 20G and 30A-30C). These results provide evidence that the unique iscDNase-seq peaks may be more likely to contribute to transcriptional regulation than the unique dscATAC-seq peaks do.

### iscDNase-seq Provide Better Prediction of Cellular Heterogeneity in Gene Expression Compared to scATAC-seq

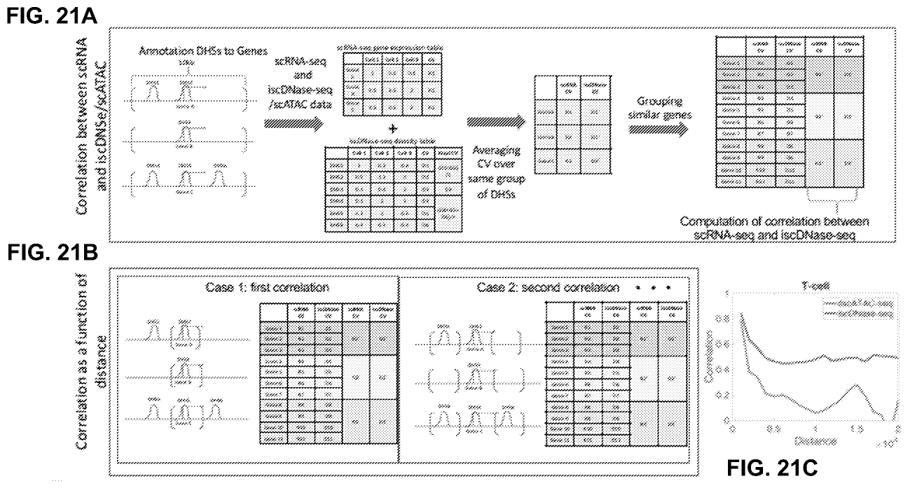

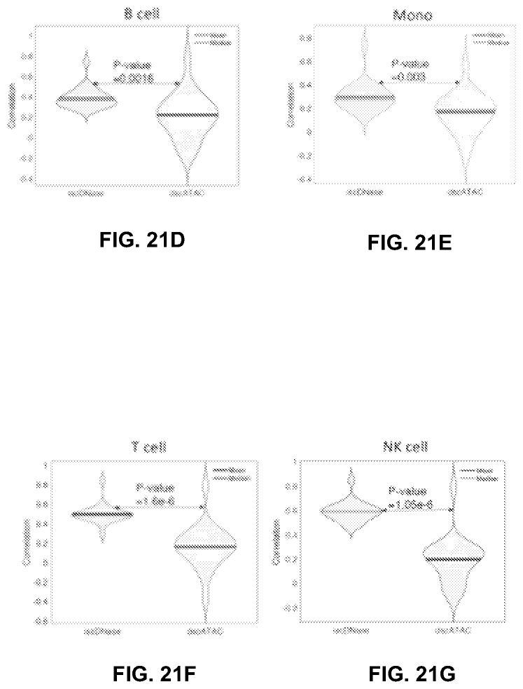

One major goal of performing single-cell experiments is to examine the cellular heterogeneity. Elucidating the relationship between cell-to-cell variation in different omics layers is critical for identifying the origins of cellular heterogeneity and understanding how different omics layers interact. Previous studies reported that cell-to-cell variation in accessibility is positively correlated with that in gene expression. However, it is not clear whether the degree of difference in detecting accessibility could affect this correlation. To address this question, the correlation between iscDNase-seq or dscATAC-seq with scRNA-seq was computed as described in FIGS. 21A and 21B.

The strategy of calculating the correlation between iscDNase-seq or dscATAC-seq with scRNA-seq is described below (FIG. 21A and 21B). DHSs were annotated to a gene if the distance between them is shorter than a threshold (e.g., 10 kb). Therefore, while computing the cell-to-cell variation in gene expression, the corresponding cell-to-cell variation in accessibility can also be computed. Note that the cell-to-cell variation is characterized by the coefficient of variation. Also, genes are aggregated into different groups based on the ranked CV in accessibility. Each group of genes are assigned with the average cell-to-cell variation in both gene expression and accessibility. Finally, the correlation between cell-to-cell variation in gene expression and accessibility over the groups of genes (FIG. 21A) is computed.

It is possible that either of the assays detects the more precise accessibility of the open chromatin regions at different distances away from TSSs. Therefore, genome regions that are 20 kb downstream and upstream of TSSs are divided into bins with equal bin size of 500 bp. For each assay, multiple correlation coefficients were computed between the variation in accessibility and gene expression, using different annotations of DHSs to TSS based on the consideration of different bins. In each calculation, only bins that have the same distance away from TSSs were considered. Finally, a set of correlation coefficients were obtained which refer to bins that are located away from TSSs with different distances (FIG. 21B). DHSs that are further away from TSSs is expected to have lower impact to the gene expression of the TSSs. Indeed, it was observed that the correlation between cell-to-cell variation in accessibility and gene expression decrease, for both iscDNase-seq and dscATAC-seq, when the distance between the considered DHSs and TSSs increases (FIG. 21C). However, the correlation between iscDNase-seq and scRNA-seq is significantly higher than that between dscATAC-seq and scRNA-seq through all distances (FIG. 21C). Furthermore, the variation in accessibility of iscDNase-seq peaks annotated to TSS is significantly better correlated with variation in gene expression than the variation measured by dscATAC-seq peaks (FIGS. 21D-21G).

### DISCUSSION

It was previously demonstrated scDNase-seq is a sensitive method for detecting genome-wide DHSs in very small number of cells or single-cells (Jin, W., et al. (2015) Genome-wide detection of DNase I hypersensitive sites in single cells and FFPE tissue samples. Nature, 528, 142-146). Furthermore, cell-to-cell variation in chromatin accessibility calculated using single-cell DHS data generated by scDNase-seq was highly correlated with that of gene expression based on scRNA-seq data. In this study, a new strategy was designed, iscDNase-seq, to dramatically improve the throughput of single-cells that can be analyzed in one experiment. iscDNase-seq is capable of analyzing tens of thousands of single-cells in one experiment, 100-fold improvement compared with the current scDNase-seq method, without the need of expensive and sophisticated equipment and accessible to most molecular biology laboratories.

Although both ATAC-seq and DNase-seq provide information on chromatin accessibility, recent studies found that DNase-seq and ATAC-seq can detect different chromatin open regions and DNase-seq is more likely to detect enhancer regions compared to ATAC-seq, providing evidence that iscDNase-seq and single cell ATAC-seq assays may detect different properties of chromatin. Indeed, the results from comparing the iscDNase-seq data and single cell ATAC-seq data indicated that the DHS regions uniquely detected by iscDNase-seq showed higher sequence conservation scores than those uniquely detected by scATAC-seq. Furthermore, it was demonstrated that the genes located near DHSs uniquely detected by iscDNase-seq exhibited higher expression levels than the genes located near DHSs uniquely detected by single cell ATAC-seq assays. These results indicated that iscDNase-seq is more likely to detect functional elements required for cell-specific gene expression than the single cell ATAC-seq assays do. Consistent with this, it was found that the correlation between the cell-to-cell variations in gene expression and DHSs detected by iscDNase-seq is also significantly higher than that between the cell-to-cell variations in gene expression and DHSs detected by single cell ATAC-seq assays. All these results together provide evidence that iscDNase-seq is an attractive alternative single cell method for single-cell epigenomics studies.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of simultaneously profiling chromatin occupancy and RNA in individual cells, comprising:
crosslinking cells of interest using a fixative agent;
performing chromatin cleavage on the cells and subjecting the cells to reverse transcription;
subjecting the cells to terminal deoxynucleotidyl transferase (TdT)-mediated oligonucleotide addition to both cDNA and chromatin cleaved ends in the presence of an oligonucleotide adaptor; or,
subjecting the cells to end repair, deoxyadenosine addition to the DNA ends, which is followed by T/A ligation of barcoded adaptors to DNA and primer-assisted ligation of the adaptors to cDNA ends)
pooling the cells from each reaction well and sorting or diluting the pooled cells into new wells, followed by one or more amplification steps; and,
subjecting the sorted cells to a library construction and sequencing;
thereby simultaneously profiling of chromatin occupancy and RNA in a single cell.

2. (canceled)

3. The method of claim 1, wherein the chromatin is cleaved by protein A-Micrococcal Nuclease (pA-MNase) or protein G-Micrococcal Nuclease (pG-MNase) fusion protein targeted by antibodies specific for each cleavage site.

4. The method of claim 1, wherein the chromatin is cleaved by one or more nucleases comprising: CRISPR-associated endonuclease (Cas), a nuclease from the Argonaute family of endonucleases, restriction enzymes, zinc-finger nucleases (ZFNs), transcription activator-like effector nucleases (TALENs), DNases, meganucleases, endo- or exo-nucleases, or combinations thereof.

5. The method of claim 1, wherein the reverse transcription is conducted in situ.

6. The method of claim 5, wherein the reverse transcription is conducted in the presence of an oligonucleotide dT primer and a mixture of primers that do not anneal to ribosomal RNA (rRNA).

7. The method of claim 5, wherein the reverse transcriptase primers comprise unique barcodes to distinguish RNAs from chromatin targets.

8. The method of claim 1, wherein the MNase-digested sites and cDNA are simultaneously tailed and ligated with oligonucleotide adaptors.

9. The method of claim 8, wherein the oligonucleotide adaptors are barcode adaptors allowing for identification of cleaved chromatin.

10. The method of claim 1, wherein the cells are sorted by flow cytometry or by dilution.

11. The method of claim 9, wherein single cells are resolved by identifying each unique combination of barcodes and indexes.

12. A method of diagnosing or prognosing an illness in an individual, comprising
obtaining a chromatin occupancy and RNA profile produced according to the method of claim 1,
wherein the cells of interest are from the individual; and,
using the chromatin occupancy and RNA profile to diagnose or prognose the illness.

13. The method of claim 12, wherein the cells are fixed with a fixative agent prior to the nuclease mediated cleavage of the cellular genome comprising comparing the chromatin occupancy and RNA profile from the individual's cells with a chromatin occupancy and RNA profile obtained from a normal individual.

14. The method of claim 12, wherein the illness is cancer.

15-22. (canceled)

23. A method of treating an individual for cancer, comprising:
a. detecting the presence of cancer in the individual using a method comprising subjecting cells from the individual to the method of claim 1; and,
b. administering to the individual a cancer therapeutic agent.

24. A method of determining cellular heterogeneity of a solid tumor sample from a patient, comprising
obtaining a chromatin occupancy and RNA profile in individual cells in the sample using a method comprising:
crosslinking the cells using a fixative agent;
performing chromatin cleavage on the cells and subjecting the cells to reverse transcription;
subjecting the cells to terminal deoxynucleotidyl transferase (TdT)-mediated oligonucleotide addition to both cDNA and chromatin cleaved ends in the presence of an oligonucleotide adaptor; or,
subjecting the cells to end repair, deoxyadenosine addition to the DNA ends, followed by T/A ligation of barcoded adaptors to DNA and primer-assisted ligation of the adaptors to cDNA ends)
pooling the cells from each reaction well and sorting or diluting the pooled cells into new wells, followed by one or more amplification steps; and, subjecting the sorted cells to a library construction and sequencing;
thereby simultaneously producing a profile of chromatin occupancy and RNA in each individual cell; and
using the chromatin and RNA profile of each cell in the tumor sample to determine the cellular heterogeneity of the tumor sample.

25. The method of claim 24, wherein the determination of the cellular heterogeneity of the tumor accurately diagnoses stages and nature of the tumor.

26-47. (canceled)

48. The method of claim 24, wherein the chromatin is cleaved by a nuclease selected from the group consisting of a protein A-Micrococcal Nuclease (pA-MNase) fusion protein targeted by an antibody specific for a cleavage site, protein G-Micrococcal Nuclease (pG-MNase) fusion protein targeted by an antibody specific for a cleavage site, a CRISPR-associated endonuclease (Cas), a nuclease from the Argonaute family of endonucleases, a restriction enzyme, a zinc-finger nuclease (ZFN), a transcription activator-like effector nuclease (TALEN), a DNase, a meganuclease, an endo- or exo-nuclease, and combinations thereof.

49. The method of claim 24, wherein the reverse transcription is conducted in situ.

50. The method of claim 49, wherein the reverse transcription is conducted in the presence of an oligonucleotide dT primer and a mixture of primers that do not anneal to ribosomal RNA (rRNA).

51. The method of claim 49, wherein the reverse transcriptase primers comprise unique barcodes to distinguish RNAs from chromatin targets.


SEQUENCE LISTING (13 sequences)

SEQ ID NO: 1 - DNA, 28 bp, Artificial Sequence
  Description of Artificial Sequence Synthetic oligonucleotide
agaaccatgtcgtcagtgtccccccccc

SEQ ID NO: 2 - DNA, 64 bp, Artificial Sequence
  Description of Artificial Sequence Synthetic oligonucleotide
  modified_base (23)..(30): a, c, t, g, unknown or other
acactgacgacatggttctacannnnnnnnagatcggaagagcacacgtctgaactccagtcac

SEQ ID NO: 3 - DNA, 31 bp, Artificial Sequence
  Description of Artificial Sequence Synthetic oligonucleotide
tgtagaaccatgtcgtcagtgtccccccccc

SEQ ID NO: 4 - DNA, 31 bp, Artificial Sequence
  Description of Artificial Sequence Synthetic oligonucleotide
gatcggaagagcgtcgtgtagggaaagagtg

SEQ ID NO: 5 - DNA, 29 bp, Artificial Sequence
  Description of Artificial Sequence Synthetic oligonucleotide
tctttccctacacgacgctcttccgatct

SEQ ID NO: 6 - DNA, 58 bp, Artificial Sequence
  Description of Artificial Sequence Synthetic primer
  modified_base (23)..(28): a, c, t, g, unknown or other
tacggtagcagagacttggtctnnnnnngtgactggagttcagacgtgtgctcttccg

SEQ ID NO: 7 - DNA, 60 bp, Artificial Sequence
  Description of Artificial Sequence Synthetic primer
  modified_base (30)..(37): a, c, t, g, unknown or other
aatgatacggcgaccaccgagatctacacnnnnnnnnacactctttccctacacgacgct

SEQ ID NO: 8 - DNA, 35 bp, Artificial Sequence
  Description of Artificial Sequence Synthetic primer
  modified_base (1)..(6): a, c, t, g, unknown or other
  modified_base (35)..(35): a, c, t, g, unknown or other
nnnnnngagcgttttttttttttttttttttttvn

SEQ ID NO: 9 - DNA, 19 bp, Artificial Sequence
  Description of Artificial Sequence Synthetic oligonucleotide
agaaccatgtcgtcagtgt

SEQ ID NO: 10 - DNA, 56 bp, Artificial Sequence
  Description of Artificial Sequence Synthetic oligonucleotide
acactgacgacatggttctacaagatcggaagagcacacgtctgaactccagtcac

SEQ ID NO: 11 - DNA, 52 bp, Artificial Sequence
  Description of Artificial Sequence Synthetic primer
aatgatacggcgaccaccgagatctacacacactctttccctacacgacgct

SEQ ID NO: 12 - DNA, 46 bp, Artificial Sequence
  Description of Artificial Sequence Synthetic primer
caagcagaagacggcatacgagattacggtagcagagacttggtct

SEQ ID NO: 13 - DNA, 31 bp, Artificial Sequence; identical to SEQ ID NO: 4
  Description of Artificial Sequence Synthetic oligonucleotide